(19) United States
(12) Patent Application Publication
(10) Pub. No.: US 2010/0280060 A1
(43) Pub. Date: Nov. 4, 2010

(54) Methods of screening and compositions for life span modulators

(75) Inventors: Ralph W. Powers, III, Washington, DC (US); Matthew R. Kaeberlein, North Bend, WA (US); Stanley Fields, Seattle, WA (US)

Correspondence Address: FENWICK & WEST LLP, SILICON VALLEY CENTER, 801 CALIFORNIA STREET, MOUNTAIN VIEW, CA, 94041, US

(73) Assignee: The University of Washington, Seattle, WA (US)

(21) Appl. No.: 12/001,889

(22) Filed: Dec. 12, 2007

Related U.S. Application Data
(63) Continuation of application No. 11/107,244, filed on Apr. 15, 2005, now abandoned.

(51) Int. Cl. A61K 31/436; A61P 35/00; A61P 25/28; A61P 9/00; A61P 25/00; A61P 25/16; A61P 3/10

(52) U.S. Cl. 514/291

(57) ABSTRACT

Identification of nucleic acids involved in life span diseases and disorders or related diseases and disorders, and the use of such methods for identifying candidate agents which modulate life span diseases and disorders or related diseases and disorders are provided. Compositions and methods for treating life span diseases and disorders or related diseases and disorders are provided. Pharmaceutical compositions for treating life span diseases and disorders or related diseases and disorders are also provided

### CROSS REFERENCE TO RELATED APPLICATIONS

This application is a continuation of U.S. application Ser. No. 11/107,244 filed on Apr. 15, 2005 the contents of which are expressly incorporated by reference herein in its entirety.

### STATEMENT OF GOVERNMENT SUPPORT

This invention was made by government support by Grant No. T32 AG00057 from the National Institutes of Health. The Government has certain rights in this invention.

### FIELD

The present invention relates to methods for identifying genes that confer longevity, methods for utilizing the identified genes to screen pharmacological agents useful for extending life spans, and related compositions comprising identified gene sequences.

### BACKGROUND

Aging is an enormous problem facing society. Age is the best predictor of risk for most of the common diseases plaguing humans, including cancer and atherosclerosis (Anisimov et al., Crit Rev Oncol Hematol. 45(3): 277-304, 2003). The relationship between aging and carcinogenesis: a critical appraisal. Crit Rev Oncol Hematol. 2003 March; 45(3):277-304). Especially in developed nations where an ever-increasing proportion of the society is elderly, the costs of aging are staggering. Besides the suffering that results as age-related maladies exhaust human lives, aging creates an enormous economic and financial strain on society (Creedon, Thought 60: 196-204, 1985; Koyama, Methods Inf Med. 2000 39: 229-32, 2000). The underlying causal mechanisms for the gradual and progressive deterioration associated with aging is poorly understood. One way toward easing this burden, and potentially preventing many age-related diseases, is by improving our understanding of the causes of the aging process at a molecular level.

Recent advances using model organisms such as mice, flies, worms, and yeast have provided some insights into the mechanisms of aging. Interestingly, this body of research indicates that many interventions that slow the rate of aging and increase life span are conserved between species. Caloric restriction (CR) is one such intervention. Despite the fact that CR has been known to extend life span for over 70 years, and can slow aging in virtually every biological system examined, the molecular basis of the life span extension afforded by CR remains unclear. Many genetic manipulations are also known to extend life span in model organisms. In general, these interventions involve the up-regulation of stress-responsive genes, and are thought to influence life span through a mechanism akin to CR.

Research in mammalian systems is most likely to be directly applicable to humans, but despite recent progress in understanding mammalian aging, rodent models are fraught with challenges to the experimenter. The ultimate marker for lifespan, death, takes approximately three years in rodents. In addition, large cohorts of aging mice are expensive to maintain, making large-scale screens very difficult.

The budding yeast, S. cerevisiae, has been used extensively as a model for cellular aging. An extremely useful feature of this organism is that it can be used to study both replicative and post-mitotic cellular aging. Replicative lifespan in yeast has been likened to stem cell replicative potential because of the common asymmetric nature of the cellular divisions. Measurement of the replicative lifespan of yeast involves counting the number of buds an individual mother cell can give off before senescing. (Mortimer and Johnston, Nature. 183: 1751-1752, 1959). Chronological lifespan in yeast has been likened to aging of post mitotic tissues. Its measurement involves growing a culture of cells to maximal density, at which point nutrients become limiting, cell division arrests, and the cells then enter a G0-like state (reviewed in Longo and Fabrizio, Cell Mol Life Sci. 59: 903-8, 2002). The fraction of cells that can reenter the cell cycle when exposed to nutrient rich media is considered the fraction viable, and individual cultures are followed longitudinally until viability is zero. Thus, in one genetic system, both types of aging can be modeled.

In yeast, two assays have been developed that serve as models for aging. Replicative life span (RLS) analysis measures the number of mitotic cycles that a cell can undergo prior to senescence, while chronological life span (CLS) analysis measures the time that a yeast cell can retain viability under non-replicative conditions. Since complex, multicellular organisms are a compilation of replicative and post-replicative cells, and aging phenotypes are evident in both cell types, both yeast assays are highly relevant.

Although the survival of yeast under nutrient deprived conditions (e.g., stationary phase) has been well described, the study of yeast chronological aging per se is a relatively new development. After exhausting available nutrients, diploid yeast may initiate a sporulation process generating haploid spores that can remain viable under adverse conditions indefinitely. Haploid strains (or diploids under conditions not suitable for sporulation) enter stationary phase, and can thus retain viability for a limited period of time (Werner-Washburne et al., Mol. Microbiol. 19: 1159-1166, 1996; Herman, Curr. Opin. Microbiol. 5: 602-607, 2002). Entry into stationary phase is accompanied by a number of cellular changes including accumulation of storage carbohydrates, cell cycle arrest, cell wall thickening, changes in gene expression, and stress resistance (Gasch and Werner-Washburne, Funct. Integr. Genomics 2: 181-192, 2002).

All of the methods for measuring chronological life span described in the literature thus far require determining viability by plating cells and counting colony forming units (CFUs), a time consuming and resource-expensive approach. In order to measure chronological life span in a high throughput manner, methods are needed that will allow the quantitative determination of CLS for each single gene deletion mutant present in the Saccharomyces cerevisiae haploid ORF deletion collection. (Winzeler et al., Science 285: 901-6, 1999) in addition to cells and collections from other species.

Analysis of yeast variants that are predisposed to longevity can yield previously uncharacterized genes that confer long life spans. An improved method for efficiently evaluating a large collection of genetic variants with differential life spans in order to identify genes that confer longevity is highly desirable. Gene products that regulate the life spans of eukaryotes can be targeted by pharmaceutical agents in order to decrease the rate of aging. Methods for screening pharmaceutical agents that can increase the life expectancy of mammals, including humans, are of intense interest to gerontologists. In addition, by slowing the rate of aging, it may be possible to delay the onset of various diseases/conditions associated with aging.

### SUMMARY

The present invention therefore provides nucleic acids encoding chronological life span proteins. The invention therefore provides methods of screening for variants. The invention further provides compounds, e.g., small organic molecules, antibodies, peptides, lipids, peptides, cyclic peptides, nucleic acids, antisense molecules, RNAi molecules, and ribozymes, that are capable of modulating chronological life span genes and gene products, e.g., inhibiting chronological life span genes. Therapeutic and diagnostic methods and reagents are also provided.

In one aspect, a highthroughput method for identifying variants to determine whether a variant within the set of variants exhibits a phenotype of interest is provided, the method comprising: providing cells in a first multiwell plate; culturing the cells in the first multiwell plate under defined environmental parameters; transferring at multiple time intervals cells from wells from the first plate into corresponding wells of at least one second multiwell plate containing fresh growth media; culturing the at least one second multiwell plate under conditions for favorable for growth; measuring the optical density (OD) of the at least one second multiwell plate after a culture period; calculating viability of the cells in the first multiwell plate based on growth of cells in the at least one second multiwell plate; and determining whether a variant within the set of variants exhibits a phenotype of interest. In another aspect, the cell is a yeast cell. In another aspect, the yeast cell is Saccharomyces cerevisiae. In another aspect, the multiwell plate comprises up to 96 wells. In another aspect, the multiwell plate comprises greater than 96 wells. In another aspect, the multiwell plate comprises up to 384 wells. In another aspect, the multiwell plate comprises greater than 384 wells. In another aspect, the multiple time intervals are daily, weekly, or monthly. In another aspect, the culturing of the at least one second plate is done under standard cell culture conditions. In another aspect, measuring the phenotype is determining a chronological life span for the cells. In another aspect the method further comprises treating the cells in the first multiwell plate with at least one compound that putatitively modulates the activity of a phenotype of interest.

In another aspect, the invention provides a method for identifying genes having life-span-regulating acitivity, the method comprising: identifying a variant having substantially greater life span than the life span of a wildtype reference, according to the method described above; and identifying a gene having life-span-regulating activity from the variant.

In another aspect, the invention provides a method of screening a test agent for an ability to modulate chronological life span comprising: providing a eukaryotic cell that expresses a chronological life span phenotype; treating the cell with at least one compound that putatively modulates the activity of the chronological life span phenotype; assaying the effect of the at least one compound that putatively modulates the activity of the chronological life span phenotype of the cell compared to the chronological life span phenotype of the cell without the at least one compound; identifying whether the at least one putative modulatory compound modulates the activity of such chronological life span phenotype. In another aspect, eukaryotic cell is selected from the group consisting of insect cells, yeast cells, worm cells and mammalian cells. In some aspects the cell is a yeast cell. In some aspects, the yeast cell is Saccharomyces cerevisiae. In another aspect, the method is a high throughput screening assay. In some aspectes, the screening comprises robotic high-throughput screening. In some aspects, the screening is performed using a multiwell plate. In some aspects, the multiwell plate comprises up to 96 wells. In other aspects, the multiwell plate comprises greater than 96 wells. In other aspects, the multiwell plate comprises up to 384 wells. In other aspects, the multiwell plate comprises greater than 384 wells.

In another aspect, the invention provides a method of screening bioactive agents comprising: a) providing a cell that expresses a chronological life span gene as set forth in Table 1 or ortholog thereof, or fragment thereof; b) adding a bioactive agent candidate to the cell; and c) determining the effect of the bioactive agent candidate on the expression of the chronological life span gene. In some aspects, the determining comprises comparing the level of expression in the absence of the bioactive agent candidate to the level of expression in the presence of the bioactive agent candidate.

In another aspect, the invention provides a method of screening for a bioactive agent capable of binding to a chronological life span extension protein, wherein the chronological life span protein is encoded by a nucleic acid encoding a gene as set forth in Table 1 or ortholog thereof, or fragment thereof, the method comprising: a) combining the chronological life span protein and a candidate bioactive agent; and b) determining the binding of the bioactive agent to the life span protein.

In another aspect, the invention provides a method for screening for a bioactive agent capable of modulating the activity of a chronological life span protein, wherein the chronological life span protein is encoded by a nucleic acid encoding a gene as set forth in Table 1 or ortholog thereof, or fragment thereof, the method comprising: a) combining the chronological life span protein and a candidate bioactive agent; and b) determining the effect of the bioactive agent on the bioactivity of the chronological life span protein.

In another aspect, the invention provides a method of evaluating the effect of a chronological life span modulating drug comprising: a) administering the drug to a mammal; b) removing a cell sample from the mammal; and c) determining the expression of a gene set forth in Table 1 or ortholog thereof. In some aspects, the method according further comprises comparing the expression profile to an expression profile of a healthy mammal.

In another aspect, the invention provides a method of diagnosing a chronological life span disease or related disorder comprising: a) determining the expression of one or more genes set forth in Table 1 or ortholog thereof, or a polypeptide encoded thereby in a first tissue type or cell of a first subject; and b) comparing the expression of the gene(s) from a second normal tissue type or cell from the first subject or a second unaffected subject; wherein a difference in the expression indicates that the first subject has a chronological life span or related disorder.

In another aspect, the invention provides a method for screening for a bioactive agent capable of interfering with the binding of a chronological life span protein or a fragment thereof and an antibody which binds to the chronological life span protein or fragment thereof, the method comprising: a) combining a chronological life span or fragment thereof, a candidate bioactive agent and an antibody which binds to the chronological life span extension protein or fragment thereof; and b) determining the binding of the chronological life span extension protein or fragment thereof and the antibody.

In another aspect, the invention provides a method for inhibiting the activity of a chronological life span protein, wherein the chronological life span protein is a gene product of a gene set forth in Table 1 or ortholog thereof, or a fragment thereof, the method comprising binding an inhibitor to the chronological life span protein. In some aspects the chronological life span genes identified by the methods of the invention include GLN3, LYS12, YG1007W, MEP2, RPP2A, MEP3, TEF4, GTR2, YGR054W, RTG2, DAL80, AGP1, GTR1, YBR077C, RPS25A, or TOR1, or ortholog thereof, or fragment thereof.

In another aspect, the invention provides a method of treating a chronological life span disease, disorder or related disease or disorder comprising administering to a subject an inhibitor of a chronological life span protein, wherein the chronological life span protein is a gene product of a gene set forth Table 1 or ortholog thereof, or a fragment thereof.

In another aspect, the invention provides a method of neutralizing the effect of a chronological life span protein, or a functional fragment thereof, comprising contacting an agent specific for the protein, or a functional fragment thereof, with the protein in an amount sufficient to effect neutralization.

In another aspect, the invention provides a method treating a chronological life span disease, disorder or related disease or disorder in a subject comprising administering to the subject a nucleic acid molecule that hybridizes under stringent conditions to a target gene as shown in Table 1 or ortholog thereof, or fragment thereof, and attenuates expression of the target gene. In some aspects, the nucleic acid molecule is an antisense oligonucleotide. In other aspects, the nucleic acid molecule is a double stranded RNA molecule. In some aspects, the nucleic acid molecule is a DNA molecule comprising a nucleotide sequence encoding an shRNA molecule. In other aspects, the double stranded RNA molecule is short interfering RNA (siRNA) or short hairpin RNA (shRNA).

In another aspect, the invention provides a method of inhibiting expression of a gene or its ortholog as shown in Table 1 comprising the steps of (i) providing a biological system in which expression of a gene shown in Table 1 or ortholog thereof to be inhibited; and (ii) contacting the system with a double stranded RNA molecule that hybridizes to a transcript encoding the protein translated from the gene; and (iii) inhibiting expression of the gene encoding the protein.

In another aspect, the invention provides a compound comprising a double stranded RNA having a nucleotide sequence that hybridizes under stringent conditions to a target gene shown in Table 1 or an ortholog thereof, and attenuates expression of said target gene. In some aspects, the double stranded RNA hybridizes to an untranslated sequence of the target gene. In other aspects, the double stranded RNA hybridizes to an intron sequence of the target gene.

In another aspect, the invention provides a compound that inhibits a chronological life span gene, the compound comprising an oligonucleotide that interacts with an ortholog of a gene shown in Table 1 having at least about 40% sequence similarity to the ortholog. In some aspects, the oligonucleotide interacts with a gene product encoded by an ortholog of a gene shown in Table 1 having at least about 40% sequence similarity to the ortholog. In other aspects, the oligonucleotide insteracts with a gene product encoded by the gene having at least about 70% sequence similarity to the ortholog. In some aspects, the at least one of: a single-stranded DNA oligonucleotide, double-stranded DNA oligonucleotide, a single-stranded RNA oligonucleotide, double-stranded RNA oligonucleotide, and modified variants of these.

In another aspect, the invention provides a biochip comprising one or more nucleic acid segments encoding the genes as shown in Table 1 or ortholog thereof, or a fragment thereof, wherein the biochip comprises fewer than 1000 nucleic acid probes. In some aspects, the probes are cDNA sequences. In other aspects, the biochip comprises a plurality of sets of probes, each set of probes complementary to subsequences from a mRNA. In some aspects, the biochip comprises a plurality of sets of probes, each set of probes complementary to subsequences from a mRNA.

In another aspect, the invention provides a method for treating a chronological life span extension disease, disorder susceptibility or related disease or disorder susceptibility comprising: providing a subject at risk of or suffering from a chronological life span disease, disorder or related disease or disorder; and administering a compound that modulates activity or abundance of one or more genes set forth in Table 1 or ortholog thereof.

In some aspects, the compound modulates the human ortholog of GLN3, LYS12, YG1007W, MEP2, RPP2A, MEP3, TEF4, GTR2, YGR054W, RTG2, DAL80, AGP1, GTR1, YBR077C, RPS25A, or TOR1, or fragment thereof.

In another aspect, the invention provides an oligonucleotide designed to specifically detect or amplify a naturally occurring polymorphic variant of a polymorphism in a coding or noncoding portion of a gene set forth in Table 1 or ortholog thereof, or a polymorphic variant of a polymorphism in a genomic region linked to such a gene, wherein the gene or a portion thereof is coincident with a chronological life span disease, disorder or related disease or disorder.

In some aspects, the gene is the human ortholog of GLN3, LYS12, YG1007W, MEP2, RPP2A, MEP3, TEF4, GTR2, YGR054W, RTG2, DAL80, AGP1, GTR1, YBR077C, RPS25A, or TOR1, or fragment thereof.

In another aspect, the invention provides a kit comprising the oligonucleotides as described above and one or more items selected from the group consisting of: packaging and instructions for use, a buffer, nucleotides, a polymerase, an enzyme, a positive control sample, a negative control sample, and a negative control primer or probe.

In another aspect, the invention provides an oligonucleotide array comprising a plurality of oligonucleotides as set forth above.

In some aspects, the oligonucleotides detect polymorphic variants at a plurality of different polymorphic sites.

In other aspects, a kit is provided comprising the oligonucleotide arrays disclosed herein and one or more items selected from the group consisting of: packaging and instructions for use, a buffer, nucleotides, a polymerase, an enzyme, a positive control sample, a negative control sample, and a negative control primer or probe.

In another aspect, the invention provides a method of evaluating the effect of a chronological life span bioactive agent comprising: a) administering the bioactive agent to a mammal; b) removing a cell sample from the mammal; and c) determining the expression profile of the cell sample. In some aspects, the method further comprises comparing the expression profile to an expression profile of a healthy individual.

In other aspects, the expression profile includes at least one GLN3, LYS12, YG1007W, MEP2, RPP2A, MEP3, TEF4, GTR2, YGR054W, RTG2, DAL80, AGP1, GTR1, YBR077C, RPS25A, and TOR1 gene, or ortholog thereof.

In another aspect, the invention provides an array of probes, comprising a support bearing a plurality of nucleic acid probes complementary to a plurality of mRNAs fewer than 1000 in number, wherein the plurality of mRNA probes includes an mRNA expressed by at least one GLN3, LYS12, YG1007W, MEP2, RPP2A, MEP3, TEF4, GTR2, YGR054W, RTG2, DAL80, AGP1, GTR1, YBR077C, RPS25A, and TOR1 gene, or ortholog thereof. In some aspects, the probes are cDNA sequences. In other aspects, the array comprises a plurality of sets of probes, each set of probes complementary to subsequences from a mRNA.

In another aspect, the invention provides a pharmaceutical composition comprising a compound as described herein and a pharmaceutically acceptable carrier.

In another aspect, the invention provides a bioactive agent that extends life span by inhibiting the TOR pathway. In some aspects, the bioactive agent is rapamycin or a rapamycin analog, derivative or related compound thereof.

In another aspects, the invention provides a bioactive agent that extends life span by inhibiting the TOR pathway in a model organism. In some aspects, the model organism is yeast. In some such aspects, the yeast is Saccharomyces cerevisiae.

In another aspect, the invention provides a chronological life span nucleic acid having a sequence at least 95% homologous to a sequence of a nucleic acid of Table 1 or ortholog thereof, or its complement. In some aspects, the invention provides a vector comprising the nucleic acid molecule as described above. In other aspects the invention provides an isolated host cell comprising the vector as described above.

In another aspect, the invention provides a method for producing a chronological life span protein, the method comprising the steps of: a) culturing the host cell as described above under conditions suitable for the expression of the polypeptide; and b) recovering the polypeptide from the host cell culture. In some aspects the host cell is a eukaryotic cell. In other aspects the host cell is a prokaryotic cell.

### BRIEF DESCRIPTION OF THE DRAWINGS

The file of this patent contains at least one drawing executed in color. Copies of this patent with color drawings will be provided by the Office upon request and payment of the necessary fee.

FIG. 1. Schematic Diagram for High throughput Chronological Life Span Analysis. A) The Aging plate—A cell culture plate is inoculated with cells and incubated throughout the experiment. Each well of the plate may contain cells treated with different drugs, with genetic modifications, or other experimental manipulations. B) Longitudinal Measurements—At multiple time intervals, small, equal volumes of cells are transferred from the cell culture plate into a corresponding well of a second plate containing fresh media. This second plate is then incubated and the optical density (OD) of each well is measured after incubation. C) Calculating Viability—The OD of each well after this out-growth period is highly correlated to the number of viable cells that originally were transferred to that well from the aging plate, which allows for determination of the percentage of cells that remain viable in each well of the aging plate.

FIG. 2. Validation Experiment Showing the Optimum Time Post-inoculation for OD Measurement. After heat shock at 55° C. for 0, 5, 10, or 15 minutes, equal numbers of cells were pinned into rich medium and incubated at 30° C. OD measurements were taken every two hours. Each point represents the average of eight individual wells. Three CFU measurements were taken per treatment and the average is shown in parenthesis. OD measurements after 23 hours of incubation are highly correlated with viability of the original culture from which cells were transferred.

FIG. 3. Yeast deletion mutants extend cellular life span. Measurements were made on five individual samples of each genotype for each time point. Deletion of genes linked to nitrogen acquisition and Tor signaling extend chronological life span.

FIG. 4. Inhibition of the Tor Pathway by Rapamycin Extends Chronological Life Span. Rapamycin is a potent pharmacological inhibitor of the Tor pathway. Administration of sub-toxic doses of rapamycin extend chronological life span.

### DETAILED DESCRIPTION

### 1. Introduction

The invention provides a number of methods, reagents, and compounds that can be used either for the treatment of a chronological life span disease or disorder or a disease or disorder associated with aging (e.g., various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease), the development of treatments for life span disorders or related disorders (e.g., various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease), the practice of the other inventive methods described herein, or for a variety of other purposes.

It is to be understood that this invention is not limited to particular methods, reagents, compounds compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Chronological life span,” abbreviated as “CLS”, refers to the time cells in a quiescent state remain viable, and CLS has been proposed as a model for the aging of post-mitotic tissues in mammals. For example, during human development, many cells of the brain exit the cell cycle and exist as non-dividing tissue; similarly yeast cells in a CLS assay are grown in culture until they exhaust nutrients, stop dividing, and exit the cell cycle. Cellular damage that regulates the life span of human brain cells, can also control the life span of yeast cells in culture. Therefore the study of mechanisms regulating yeast CLS may inform future work on life span of human cell types. “Chronological life span” and “chronological life span phenotype” means the length of time a cell maintains viability (the ability to re-enter the cell cycle) during quiescence.

“Chronological life span protein” or “CLS” protein or fragment thereof, or nucleic acid encoding “chronological life span” or “CLS” or a fragment thereof refer to nucleic acids and polypeptide polymorphic variants, alleles, mutants, and interspecies homologs that: (1) have an amino acid sequence that has greater than about 40% amino acid sequence identity, 45%, 50%, 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by a CLS nucleic acid or amino acid sequence of an CLS protein, e.g., a CLS protein as shown in Table 1 or ortholog as shown Table 2; (2) specifically bind to antibodies, e.g., polyclonal antibodies, raised against an immunogen comprising an amino acid sequence of a CLS protein, e.g a CLS protein as shown in Table 1 or ortholog as shown Table 2, immunogenic fragments thereof, and conservatively modified variants thereof; (3) specifically hybridize under stringent hybridization conditions to an anti-sense strand corresponding to a nucleic acid sequence encoding a CLS protein, e.g., CLS protein (Table 1) or ortholog as shown Table 2, and conservatively modified variants thereof; (4) have a nucleic acid sequence that has greater than about 40% amino acid sequence identity, 45%, 50%, 55%, 60%, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to a CLS nucleic acid, e.g., a CLS protein as shown in Table 1 or ortholog as shown Table 2.

A CLS polynucleotide or polypeptide sequence is typically from a mammal including, but not limited to, primate, e.g., human; rodent, e.g., rat, mouse, hamster; cow, pig, horse, sheep, or any mammal. Other CLS polynucleotide or polypeptide sequences are from other organisms, including yeast (e.g., Saccharomyces cerevisiae; also referred to as S. cerevisiae), worms, and insects. The nucleic acids and proteins of the invention include both naturally occurring or recombinant molecules.

The terms “CLS” protein or a fragment thereof, or a nucleic acid encoding “CLS” protein or a fragment thereof refer to nucleic acid and polypeptide polymorphic variants, alleles, mutants, and interspecies homologs that: (1) have an amino acid sequence that has greater than about 40% amino acid sequence identity, 45%, 50%, 55%, 60%, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by as shown in Table 1 or Table 2; (2) specifically bind to antibodies, e.g., polyclonal antibodies, raised against an immunogen comprising an amino acid sequence encoded by a CLS protein as shown in Table 1 or ortholog as shown Table 2, immunogenic fragments thereof, and conservatively modified variants thereof; (3) specifically hybridize under stringent hybridization conditions to an anti-sense strand corresponding to a nucleic acid sequence encoding a CLS protein, e.g., a CLS protein as shown in Table 1 or ortholog as shown Table 2, or their complements, and conservatively modified variants thereof; (4) have a nucleic acid sequence that has greater than about 40% amino acid sequence identity, 45%, 50%, 55%, 60%, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to the genes and their representative sequences as a CLS protein as shown in Table 1 or ortholog as shown Table 2 or their complements.

Exemplary chronological life span genes are listed in Table 1 and interspecies orthologs for these genes are listed in Table 2.

The GenPept accession number for gln3 is AAB64575, and GenBank accession number for exemplary nucleotide and amino acid sequences is U18796.1 (see, e.g., Dietrich et al., Nature 387 (6632 Suppl): 78-81, 1997).

The GenPept accession number for lys12 is CAA86700, and GenBank accession number for exemplary nucleotide and amino acid sequences is 246728.1.

The GenPept accession number for yg1007w is CAA96707.1, and GenBank accession number for exemplary nucleotide and amino acid sequences is Z72529.1.

The GenPept accession number for mep2 is CAA96025.1, and GenBank accession number for exemplary nucleotide and amino acid sequences is Z71418.1.

The GenPept accession number for rpp2a is CAA99041.1, and GenBank accession number for exemplary nucleotide and amino acid sequences is Z74781.1.

The GenPept accession number for mep3 is AAB68278.1, and GenBank accession number for exemplary nucleotide and amino acid sequences is U40829.1 (see, e.g., Bussey et al., Nature 387 (6632 Suppl): 103-105, 1997)

The GenPept accession number for tef4 is CAA81919.1, and GenBank accession number for exemplary nucleotide and amino acid sequences is Z28081.1.

The GenPept accession number for gtr2 is BAA28781.1, and GenBank accession number for exemplary nucleotide and amino acid sequences is AB015239.1 (see, e.g., Nakashima et al., Genetics 152: 853-867, 1999).

The GenPept accession number for ygr054w is CAA97054.1, and GenBank accession number for exemplary nucleotide and amino acid sequences is Z72839.1.

The GenPept accession number for rtg2 is CAA96972.1, and GenBank accession number for exemplary nucleotide and amino acid sequences is Z72774.1.

The GenPept accession number for dal80 is CAA82107.1, and GenBank accession number for exemplary nucleotide and amino acid sequences is Z28259.1.

The GenPept accession number for agp1 is CAA42360.2, and GenBank accession number for exemplary nucleotide and amino acid sequences is X59720.2 (see, e.g., Rad et al., Yeast 7: 533-538, 1991; and Biteau et al., Yeast 8: 61-70, 1992).

The GenPept accession number for gtr1 is CAA89159.1, and GenBank accession number for exemplary nucleotide and amino acid sequences is Z49218.1.

The GenPept accession number for ybr077c is CAA85021.1, and GenBank accession number for exemplary nucleotide and amino acid sequences is Z35946.1 (see, e.g., Feldmann et al., EMBO J. 13: 5795-5809, 1994).

The GenPept accession number for rps25a is CAA97010.1, and GenBank accession number for exemplary nucleotide and amino acid sequences is Z72812.1.

The GenPept accession number for tor1 is AAB39292.1, and GenBank accession number for exemplary nucleotide and amino acid sequences is L47993.1 (see, e.g., Huang et al., Yeast. 12: 869-875, 1996). “TOR” refers to a 280-300 kD peptide belonging to the phosphoinositide (PI) 3-kinase family, which phosphorylate proteins on serine or threonine residues. TOR is a highly conserved protein kinase found in both prokaryotes and eukaryotes. For example, Raught et al., Proc. Natl. Acad. Sci U.S.A. 98: 7037, 2001, describe homologues of TOR protein found in Saccharomyces cerevisiae, Schizosaccharomyces pombe, Drosophila melanogaster and other Metazoans, and mammals. A single mammalian TOR protein has been cloned from several species. (Raught et al., Proc. Natl. Acad. Sci U.S.A. 98: 7037, 2001). In a preferred embodiment, TOR is isolated from rat brain tissue using an ion-exchange column, and fraction purification. However, native TOR is easily isolated from a variety of tissues using a variety of techniques by those of skill in the art. By way of example only, bovine testes is another source for isolating native TOR protein. Additionally, one of skill in the art will readily isolate TOR proteins from other species for use within the spirit of the present invention. As used in the following description, “TOR” refers to any and all proteins in this described family, including but not limited to dTOR, mTOR, TOR1, TOR2, RAFT and others. Many key signaling molecules are conserved from yeast to man. mTOR is a protein kinase involved in nutrient and growth factor signaling in humans. Signaling downstream of the TOR kinase pathways in yeast and humans regulates the nuclear localization of several transcription factors in response to the carbon and nitrogen sources in the nutritional environment.

“Senescence” refers to a mitotically-arrested state in which a cell or an organism may be metabolically active but is incapable of further cell division. In yeast, one cause of senescence is the accumulation of ribosomal DNA (“rDNA”) circles. In mammals, telomere shortening is one mechanism by which cells senesce. Markers for senescence in multicellular organisms include: increase in cell size, shortening in telomere-length, increase in senescence-associated beta-galactosidase (“SA-beta-gal”) expression, and altered patterns of gene expression. Assays that can detect such markers are well-known in the art. For example, senescence can be detected in cultured cells and tissue sections of organisms at pH 6 by histochemical detection of SA-beta-gal activity present only in senescent cells and not in pre-senescent, quiescent, or immortal cells. Various methods for detecting telomeres and for measuring telomere length are known, including Southern analysis of terminal restriction fragments (“TRF”) obtained by digestion of genomic DNA using frequently cutting restriction enzymes. The TRFs containing DNA with uniform telomeric repeats (TTAGGG) and degenerate repeats are separated by gel electrophoresis, blotted, and visualized directly or indirectly by hybridization with labeled oligonucleotides complementary to the telomeric-repeat sequence. “Quiescence,” differs from “senescence” in that cells can retain the ability to re-enter the cell cycle during quiescence.

“Cell culture” refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

“Standard growth conditions”, as used herein, refers to culturing of cells (e.g., mammalian cells) at 37° C., in a standard atmosphere comprising 5% CO₂. Relative humidity is maintained at about 100%. While the foregoing the conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like. For example, “standard growth conditions” for yeast (e.g., S. cerevisiae) include 30° C. and generally under regular atmospheric conditions (less than 0.5% CO₂, approximately 20% O₂, approximately 80% N₂) at a relative humidity at about 100%. The term “defined environmental parameters” with respect to culturing cells is known to one of skill in the art to include media composition, temperature, pressure, and cell density. For example, yeast cells could be cultured in yeast standard media (1% yeast extract; 2% bactopeptone and 2% glucose; yeast standard media is known to one of skill in the art and is often referred to as “YPD”) at 2×10⁸ cells/mL at 42° C. to assess survival at elevated temperature.

“Substantially greater” refers to a measured life span of an organism, such as a variant, that is at least 20%, 25%, 30%, 35%, 40%, 45%, 50%, 55%, 60%, 65%, 70%, 75%, 80%, 85%, 90%, 95%, or 100% greater than that of a reference organism, such as a wildtype. “Substantially less” refers to a measured life span of an organism, such as a variant, that is at least 20%, 25%, 30%, 35%, 40%, 45%, 50%, 55%, 60%, 65%, 70%, 75%, 80%, 85%, 90%, 95%, or 100% less than that of a reference organism, such as a wildtype.

“Longevity-promoting” can refer to a substantial increase in a life span of an organism by at least 10%, 15%, 20%, 25%, 30%, 35%, 40%, 45%, 50%, 55%, 60%, 65%, 70%, 75%, 80%, 85%, 90%, 95%, or 100%, from an exposure to a compound. “Longevity-inhibiting” can refer to a substantial decrease in a life span of an organism by at least 10%, 15%, 20%, 25%, 30%, 35%, 40%, 45%, 50%, 55%, 60%, 65%, 70%, 75%, 80%, 85%, 90%, 95%, or 100%, from an exposure to a compound.

“Gene” refers to a unit of inheritable genetic material found in a chromosome, such as in a human chromosome. Each gene is composed of a linear chain of deoxyribonucleotides which can be referred to by the sequence of nucleotides forming the chain. Thus, “sequence” is used to indicate both the ordered listing of the nucleotides which form the chain, and the chain which has that sequence of nucleotides. The term “sequence” is used in the same way in referring to RNA chains, linear chains made of ribonucleotides. The gene includes regulatory and control sequences, sequences which can be transcribed into an RNA molecule, and can contain sequences with unknown function. Some of the RNA products (products of transcription from DNA) are messenger RNAs (mRNAs) which initially include ribonucleotide sequences (or sequence) which are translated into a polypeptide and ribonucleotide sequences which are not translated. The sequences which are not translated include control sequences, introns and sequences with unknowns function. It can be recognized that small differences in nucleotide sequence for the same gene can exist between different persons, or between normal cells and cancerous cells, without altering the identity of the gene.

“Gene expression pattern” means the set of genes of a specific tissue or cell type that are transcribed or “expressed” to form RNA molecules. Which genes are expressed in a specific cell line or tissue can depend on factors such as tissue or cell type, stage of development or the cell, tissue, or target organism and whether the cells are normal or transformed cells, such as cancerous cells. For example, a gene can be expressed at the embryonic or fetal stage in the development of a specific target organism and then become non-expressed as the target organism matures. Alternatively, a gene can be expressed in liver tissue but not in brain tissue of an adult human.

“Differential expression” refers to both quantitative as well as qualitative differences in the temporal and tissue expression patterns of a gene or a protein. For example, a differentially expressed gene can have its expression activated or completely inactivated in normal versus disease conditions. Such a qualitatively regulated gene can exhibit an expression pattern within a given tissue or cell type that is detectable in either control or disease conditions, but is not detectable in both. Differentially expressed genes can represent “profile genes,” or “target genes” and the like.

Similarly, a differentially expressed protein can have its expression activated or completely inactivated in normal versus disease conditions. Such a qualitatively regulated protein can exhibit an expression pattern within a given tissue or cell type that is detectable in either control or disease conditions, but is not detectable in both. Moreover, differentially expressed genes can represent “profile proteins”, “target proteins” and the like.

Differentially expressed genes can represent “expression profile genes”, which includes “target genes”. “Expression profile gene,” as used herein, refers to a differentially expressed gene whose expression pattern can be used in methods for identifying compounds useful in the modulation of lifespan extension or activity, or the treatment of disorders, or alternatively, the gene can be used as part of a prognostic or diagnostic evaluation of lifespan disorders, e.g., diseases or disorders associated with aging including various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease. For example, the effect of the compound on the expression profile gene normally displayed in connection with a particular state, for example, can be used to evaluate the efficacy of the compound to modulate that state, or preferably, to induce or maintain that state. Such assays are further described below. Alternatively, the gene can be used as a diagnostic or in the treatment of lifespan disorders as also further described below. In some instances, only a fragment of an expression profile gene is used, as further described below.

“Expression profile,” as used herein, refers to the pattern of gene expression generated from two up to all of the expression profile genes which exist for a given state. As outlined above, an expression profile is in a sense a “fingerprint” or “blueprint” of a particular cellular state; while two or more states have genes that are similarly expressed, the total expression profile of the state will be unique to that state. A “fingerprint pattern”, as used herein, refers to a pattern generated when the expression pattern of a series (which can range from two up to all the fingerprint genes that exist for a given state) of fingerprint genes is determined. A fingerprint pattern also can be referred to as an “expression profile”. A fingerprint pattern or expression profile can be used in the same diagnostic, prognostic, and compound identification methods as the expression of a single fingerprint gene. The gene expression profile obtained for a given state can be useful for a variety of applications, including diagnosis of a particular disease or condition and evaluation of various treatment regimes. In addition, comparisons between the expression profiles of different lifespan disorders can be similarly informative. An expression profile can include genes which do not appreciably change between two states, so long as at least two genes which are differentially expressed are represented. The gene expression profile can also include at least one target gene, as defined below. Alternatively, the profile can include all of the genes which represent one or more states. Specific expression profiles are described below.

Gene expression profiles can be defined in several ways. For example, a gene expression profile can be the relative transcript level of any number of particular set of genes. Alternatively, a gene expression profile can be defined by comparing the level of expression of a variety of genes in one state to the level of expression of the same genes in another state. For example, genes can be either upregulated, downregulated, or remain substantially at the same level in both states.

A “target gene” refers to a nucleic acid, often derived from a biological sample, to which an oligonucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The target nucleic acid can also refer to the specific subsequence of a larger nucleic acid to which the probe is directed or to the overall sequence (e.g., gene or mRNA) whose expression level it is desired to detect. A “target gene”, therefore, refers to a differentially expressed gene in which modulation of the level of gene expression or of gene product activity prevents and/or ameliorates a lifespan disease or disorder. Thus, compounds that modulate the expression of a target gene, the target gene, or the activity of a target gene product can be used in the diagnosis, treatment or prevention lifespan diseases. Particular target genes of the present invention is shown in Table 1 and Table 2.

A “target protein” refers to an amino acid or protein, often derived from a biological sample, to which a protein-capture agent specifically hybridizes or binds. It is either the presence or absence of the target protein that is to be detected, or the amount of the target protein that is to be quantified. The target protein has a structure that is recognized by the corresponding protein-capture agent directed to the target. The target protein or amino acid can also refer to the specific substructure of a larger protein to which the protein-capture agent is directed or to the overall structure (e.g., gene or mRNA) whose expression level it is desired to detect.

A “differentially expressed gene transcript”, as used herein, refers to a gene, including an chronological life span gene, transcript that is found in different numbers of copies in different cell or tissue types of an organism having a chronological life span disease or disorder or a disease or disorder associated with aging, including various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease, compared to the numbers of copies or state of the gene transcript found in the cells of the same tissue in a healthy organism, or in the cells of the same tissue in the same organism. Multiple copies of gene transcripts can be found in an organism having a chronological life span disease or disorder or a disease or disorder associated with aging, while fewer copies of the same gene transcript are found in a healthy organism or healthy cells of the same tissue in the same organism, or vice-versa.

A “differentially expressed gene,” can be a target, fingerprint, or pathway gene. For example, a “fingerprint gene”, as used herein, refers to a differentially expressed gene whose expression pattern can be used as a prognostic or diagnostic marker for the evaluation of chronologcial life span diseases or disorders, or which can be used to identify compounds useful for the treatment of such diseases or disorders or a disease or disorder associated with aging. For example, the effect of a compound on the fingerprint gene expression pattern normally displayed in connection with chronological life span diseases or disorders or diseases or disorders associated with aging, can be used to evaluate the efficacy, such as potency, of the compound as chronologcial life span treatment, or can be used to monitor patients undergoing clinical evaluation for the treatment of such a disease or disorder.

“Ortholog” refers to an evolutionarily conserved bio-molecule represented in a species other than the organism in which a reference sequence is identified, and contains a nucleic-acid or amino-acid sequence that is homologous to the reference sequence. To determine the degree of homology between a reference sequence and a sequence in question, two nucleic-acid sequences or two amino-acid sequences are compared. Homology can be defined by percentage identity or by percentage similarity. Percentage identity correlates with the proportion of identical amino-acid residues shared between two sequences compared in an alignment. Percentage similarity correlates with the proportion of amino-acid residues having similar structural properties that is shared between two sequences compared in an alignment. Percentages of similarity and identity can be calculated over a portion of the primary structure and not over the entire gene/protein sequence. For example, amino-acid residues having similar structural properties can be substituted for one another, such as the substitutions of analogous hydrophilic amino-acid residues, and the substitution of analogous hydrophobic amino-acid residues. Percentages of similarity and identity can be calculated over a portion of the primary structure and not over the entire gene/protein sequence. For the present disclosure, an ortholog or an orthologous sequence is defined as a homologous molecule or a sequence having life-span-regulating activity and a sequence identity of at least about 20%, 25%, 30%, 35%, 40%, 45%, 50%, 55%, 60%, 65%, 70%, 75%, 80%, 85%, 90%, and 95%. Alternatively, an ortholog is defined as a homologous molecule or sequence having life-span-regulating activity and a sequence similarity of at least about 40%, 40%, 45%, 50%, 55%, 60%, 65%, 70%, 75%, 80%, 85%, 90%, and 95%.

It is further contemplated that “ortholog” is a polypeptide or nucleic acid molecule of an organism that is highly related to a reference protein, or nucleic acid sequence, from another organism. An ortholog is functionally related to the reference gene, protein or nucleic acid sequence. In other words, the ortholog and its reference molecule would be expected to fulfill similar, if not equivalent, functional roles in their respective organisms. It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Such functionally important domains may be defined by genetic mutations or by structure-function assays. Orthologs can be identified using methods provided herein. The functional role of an ortholog may be assayed using methods well known to the skilled artisan, and described herein. For example, function might be assayed in vivo or in vitro using a biochemical, immunological, or enzymatic assay; transformation rescue, or for example, in a nematode bioassay for the effect of gene inactivation on nematode phenotype. Alternatively, bioassays may be carried out in tissue culture; function can also be assayed by gene inactivation (e.g., by RNAi, siRNA, or gene knockout), or gene over-expression, as well as by other methods. Exemplary orthologs for the genes of the invention are shown in Table 2.

“Paralogs” are distinct but structurally related proteins made by an organism. Paralogs are believed to arise through gene duplication.

“Variant” may refer to an organism with a particular genotype in singular form, a set of organisms with different genotypes in plural form, and also to alleles of any gene identifiable by methods of the present invention. For example, the term “variants” includes various alleles that may occur at high frequency at a polymorphic locus, and includes organisms containing such allelic variants. The term “variant” includes various “strains” and various “mutants.”

“Strains” refers to genetic variants that arise in a population, spontaneously and non-spontaneously, by acquiring a mutation or change in genomic DNA. Different strains are genotypically different with respect to at least one gene, gene regulatory element, or other non-coding element. “Strain” can be used to refer to different laboratory-generated strains and to various mutant lines that arise spontaneously in a population.

A “wild type chronological life span protein” or “native chronological life span protein” comprises a polypeptide having the same amino acid sequence as a chronological life span protein derived from nature. Thus, a wild type chronological life span protein can have the amino acid sequence of a naturally occurring rat chronological life span protein, murine chronological life span protein, human chronological life span protein, or chronological life span protein from any other mammalian species. Such wild type chronological life span polypeptides can be isolated from nature or can be produced by recombinant or synthetic means. The term “wild type chronological life span protein” specifically encompasses naturally-occurring truncated forms of the chronological life span protein, naturally-occurring variant forms (e.g., alternatively spliced forms), and naturally-occurring allelic variants of the particular chronological life span protein. Table 1 and Table 2 provide a listing of identified genes, including various exemplary orthologs.

“Patient”, “subject” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” or “treatment” includes the administration of the compositions, compounds or agents of the present invention to prevent or delay the onset of the symptoms, complications, or biochemical indicia of a disease, alleviating or ameliorating the symptoms or arresting or inhibiting further development of the disease, condition, or disorder (e.g., diseases/conditions associated with aging, including various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease). “Treating” further refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a chronological life span disease or related disease or a disease or disorder associated with aging. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a disease or disorder associated with aging but does not yet experience or exhibit symptoms, inhibiting the symptoms of a disease or disorder (slowing or arresting its development), providing relief from the symptoms or side-effects of a disease (including palliative treatment), and relieving the symptoms of a disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

“Concomitant administration” of a known drug with a compound of the present invention means administration of the drug and the compound at such time that both the known drug and the compound will have a therapeutic effect or diagnostic effect. Such concomitant administration can involve concurrent (i.e., at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

In general, the phrase “well tolerated” refers to the absence of adverse changes in health status that occur as a result of the treatment and would affect treatment decisions.

“Inhibitors,” “activators,” and “modulators” of chronological life span genes and their gene products in cells are used to refer to inhibitory, activating, or modulating molecules, respectively, identified using in vitro and in vivo assays for binding or signaling, e.g., ligands, agonists, antagonists, and their homologs and mimetics. The term “modulator” includes inhibitors and activators. Inhibitors are agents that, e.g., bind to, partially or totally block stimulation, decrease, prevent, delay activation, inactivate, desensitize, or down regulate the activity of chronological life span genes, e.g., antagonists. Activators are agents that, e.g., bind to, stimulate, increase, open, activate, facilitate, enhance activation, sensitize or up regulate the activity of chronological life span genes, e.g., agonists. Modulators include agents that, e.g., alter the interaction of chronological life span gene or gene product with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring activated chronological life span disorder ligands, e.g., with altered activity, as well as naturally occurring and synthetic ligands, antagonists, agonists, small chemical molecules and the like. Such assays for inhibitors and activators include, e.g., applying putative modulator compounds to a cell expressing a chronological life span receptor and then determining the functional effects on chronological life span receptor signaling. Samples or assays comprising activated chronological life span receptor that are treated with a potential activator, inhibitor, or modulator are compared to control samples without the inhibitor, activator, or modulator to examine the extent of inhibition. Control samples (untreated with inhibitors) can be assigned a activity value of 100%. Inhibition of activated samples is achieved when the activity value relative to the control is about 80%, optionally 50% or 25-0%. Activation of sample is achieved when the activity value relative to the control is 110%, optionally 150%, optionally 200-500%, or 1000-3000% higher.

In the case of treating chronological life span diseases or conditions or diseases or conditions associated with aging (e.g., such as Alzheimer's disease, Pick's disease, Huntington's disease, Parkinson's disease, adult onset myotonic dystrophy, multiple sclerosis, and adult onset leukodystrophy disease), an inhibitor can be desired in order to prevent the expression of certain genes. In one embodiment an inhibitor of the invention includes a molecule which inhibits the genes disclosed herein. In another embodiment, an inhibitor of the invention includes a molecule which inhibits a chronological life span protein as defined herein, at the nucleic acid or protein level. In some chronological disorders or related disorders, however, specific gene activity or gene upregulation can be desired. Methods of inhibiting or enhancing expression are further described below.

The ability of a molecule to bind to a chronological life span-related receptor can be determined, for example, by the ability of the putative ligand to bind to chronological life span-related immunoadhesin coated on an assay plate. Specificity of binding can be determined by comparing binding to non-chronological life span-related receptor.

In one embodiment, antibody binding to a chronological life span-related receptor can be assayed by either immobilizing the ligand or the receptor. For example, the assay can include immobilizing a chronological life span-related receptor fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

“Epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

An intact “antibody” comprises at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) through cellular receptors such as Fc receptors (e.g., FcγRI, FcγRIIa, FcγRIIb, FcγRIII, and FcRη) and the first component (Clq) of the classical complement system. The term antibody includes antigen-binding portions of an intact antibody that retain capacity to bind the antigen. Examples of antigen binding portions include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341: 544-546, 1989), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al., Science 242: 423-426, 1988; and Huston et al., Proc. Natl. Acad. Sci. U.S.A. 85: 5879-5883, 1988). Such single chain antibodies are included by reference to the term “antibody” Fragments can be prepared by recombinant techniques or enzymatic or chemical cleavage of intact antibodies.

“Human sequence antibody” includes antibodies having variable and constant regions (if present) derived from human immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). However, the term “human sequence antibody”, as used herein, is not intended to include antibodies in which entire CDR sequences sufficient to confer antigen specificity and derived from the germline of another mammalian species, such as a mouse, have been grafted onto human framework sequences (i.e., humanized antibodies).

“Monoclonal antibody” or “monoclonal antibody composition” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Diclonal antibody” refers to a preparation of at least two antibodies to an antigen. Typically, the different antibodies bind different epitopes.

“Oligoclonal antibody” refers to a preparation of 3 to 100 different antibodies to an antigen. Typically, the antibodies in such a preparation bind to a range of different epitopes.

“Polyclonal antibody” refers to a preparation of more than 1 (two or more) different antibodies to an antigen. Such a preparation includes antibodies binding to a range of different epitopes.

“Recombinant human antibody” includes all human sequence antibodies of the invention that are prepared, expressed, created or isolated by recombinant means, such as antibodies isolated from an animal (e.g., a mouse) that is transgenic for human immunoglobulin genes (described further below); antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

A “heterohybrid antibody” refers to an antibody having a light and heavy chains of different organismal origins. For example, an antibody having a human heavy chain associated with a murine light chain is a heterohybrid antibody.

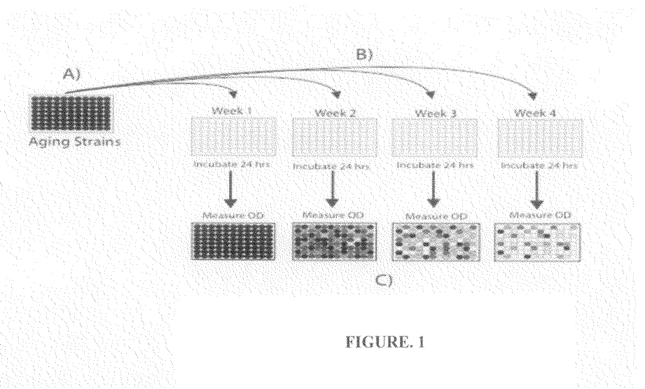

“Substantially pure” or “isolated” means an object species (e.g., an antibody of the invention) has been identified and separated and/or recovered from a component of its natural environment such that the object species is the predominant species present (i.e., on a molar basis it is more abundant than any other individual species in the composition); a “substantially pure” or “isolated” composition also means where the object species comprises at least about 50 percent (on a molar basis) of all macromolecular species present. A substantially pure or isolated composition can also comprise more than about 80 to 90 percent by weight of all macromolecular species present in the composition. An isolated object species (e.g., antibodies of the invention) can also be purified to essential homogeneity (contaminant species cannot be detected in the composition by conventional detection methods) wherein the composition consists essentially of derivatives of a single macromolecular species. For example, an isolated antibody to any one chronological gene product as shown in FIG. 1 can be substantially free of other antibodies that lack binding to that particular gene product and bind to a different antigen. Further, an isolated antibody that specifically binds to an epitope, isoform or variant of a chronological life span protein may, however, have cross-reactivity to other related antigens, e.g., from other species (e.g., chronological life span species homologs). Moreover, an isolated antibody of the invention be substantially free of other cellular material (e.g., non-immunoglobulin associated proteins) and/or chemicals.

“Specific binding” refers to preferential binding of an antibody to a specified antigen relative to other non-specified antigens. The phrase “specifically (or selectively) binds” to an antibody refers to a binding reaction that is determinative of the presence of the protein in a heterogeneous population of proteins and other biologics. Typically, the antibody binds with an association constant (Kₐ) of at least about 1×10⁶ M⁻¹ or 10⁷ M⁻¹, or about 10⁸ M⁻¹ to 10⁹ M⁻¹, or about 10¹⁰ M⁻¹ to 10¹¹ M⁻¹ or higher, and binds to the specified antigen with an affinity that is at least two-fold greater than its affinity for binding to a non-specific antigen (e.g., BSA, casein) other than the specified antigen or a closely-related antigen. The phrases “an antibody recognizing an antigen” and “an antibody specific for an antigen” are used interchangeably herein with the term “an antibody which binds specifically to an antigen”. A predetermined antigen is an antigen that is chosen prior to the selection of an antibody that binds to that antigen.

“Specifically bind(s)” or “bind(s) specifically” when referring to a peptide refers to a peptide molecule which has intermediate or high binding affinity, exclusively or predominately, to a target molecule. The phrases “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore and Western blot are used to identify peptides that specifically react with the antigen. Typically a specific or selective reaction will be at least twice background signal or noise and more typically more than 10 times background.

“High affinity” for an antibody refers to an equilibrium association constant (Kₐ) of at least about 10⁷M⁻¹, at least about 10⁸M⁻¹, at least about 10⁹M⁻¹, at least about 10¹⁰M⁻¹, at least about 10¹¹M⁻¹, or at least about 10¹²M⁻¹ or greater, e.g., up to 10¹³M⁻¹ or 10¹⁴M⁻¹ or greater. However, “high affinity” binding can vary for other antibody isotypes.

“Kₐ”, as used herein, is intended to refer to the equilibrium association constant of a particular antibody-antigen interaction. This constant has units of 1/M.

“K_{d}”, as used herein, is intended to refer to the equilibrium dissociation constant of a particular antibody-antigen interaction. This constant has units of M.

The term “kₐ”, as used herein, is intended to refer to the kinetic association constant of a particular antibody-antigen interaction. This constant has units of 1/Ms.

The term “k_{d}”, as used herein, is intended to refer to the kinetic dissociation constant of a particular antibody-antigen interaction. This constant has units of 1/s.

“Particular antibody-antigen interactions” refers to the experimental conditions under which the equilibrium and kinetic constants are measured.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG₁, IgG₂, IgG₃ and IgG₄) and IgA (e.g., IgA₁ and IgA₂)

“Isotype switching” refers to the phenomenon by which the class, or isotype, of an antibody changes from one Ig class to one of the other Ig classes/

“Nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence region in the transgene. Non-classical isotype switching can occur by, for example, homologous recombination between human σ_{μ} and human Σ_{μ} (δ-associated deletion). Alternative non-classical switching mechanisms, such as intertransgene and/or interchromosomal recombination, among others, can occur and effectuate isotype switching.

“Switch sequence” refers to those DNA sequences responsible for switch recombination. A “switch donor” sequence, typically a μ switch region, are 5′ (i.e., upstream) of the construct region to be deleted during the switch recombination. The “switch acceptor” region are between the construct region to be deleted and the replacement constant region (e.g., γ, ε, and alike). As there is no specific site where recombination always occurs, the final gene sequence is not typically predictable from the construct.

“Glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the non-human transgenic animal, when one of ordinary skill in the art would recognize the glycosylation pattern of the heterologous antibody as being more similar to said pattern of glycosylation in the species of the non-human transgenic animal than to the species from which the CH genes of the transgene were derived.

“Naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Immunoglobulin locus” refers to a genetic element or set of linked genetic elements that comprise information that can be used by a B cell or B cell precursor to express an immunoglobulin peptide. This peptide can be a heavy chain peptide, a light chain peptide, or the fusion of a heavy and a light chain peptide. In the case of an unrearranged locus, the genetic elements are assembled by a B cell precursor to form the gene encoding an immunoglobulin peptide. In the case of a rearranged locus, a gene encoding an immunoglobulin peptide is contained within the locus.

“Rearranged” refers to a configuration of a heavy chain or light chain immunoglobulin locus wherein a V segment is positioned immediately adjacent to a D-J or J segment in a conformation encoding essentially a complete VH or VL domain, respectively. A rearranged immunoglobulin gene locus can be identified by comparison to germline DNA; a rearranged locus has at least one recombined heptamer/nonamer homology element.

“Unrearranged” or “germline configuration” in reference to a V segment refers to the configuration wherein the V segment is not recombined so as to be immediately adjacent to a D or J segment.

“Nucleic acid” or “nucleic acid molecule” refer to a deoxyribonucleotide or ribonucleotide polymer in either single- or double-stranded form, and unless otherwise limited, can encompass known analogs of natural nucleotides that can function in a similar manner as naturally occurring nucleotides.

“Isolated nucleic acid” in reference to nucleic acids encoding antibodies or antibody portions (e.g., VH, VL, CDR3) that bind to the antigen, is intended to refer to a nucleic acid in which the nucleotide sequences encoding the antibody or antibody portion are free of other nucleotide sequences encoding antibodies or antibody portions that bind antigens other than, for example, a chronological life span protein, which other sequences can naturally flank the nucleic acid in human genomic DNA.

“Substantially identical,” in the context of two nucleic acids or polypeptides refers to two or more sequences or subsequences that have at least about 80%, about 90%, about 95% or higher nucleotide or amino acid residue identity, when compared and aligned for maximum correspondence, as measured using the following sequence comparison method and/or by visual inspection. Such “substantially identical” sequences are typically considered to be homologous. The “substantial identity” can exist over a region of sequence that is at least about 50 residues in length, over a region of at least about 100 residues, or over a region at least about 150 residues, or over the full length of the two sequences to be compared. As described below, any two antibody sequences can only be aligned in one way, by using the numbering scheme in Kabat. Therefore, for antibodies, percent identity has a unique and well-defined meaning.

Amino acids from the variable regions of the mature heavy and light chains of immunoglobulins are designated Hx and Lx respectively, where x is a number designating the position of an amino acid according to the scheme of Kabat, Sequences of Proteins of Immunological Interest (National Institutes of Health, Bethesda, Md., 1987 and 1991). Kabat lists many amino acid sequences for antibodies for each subgroup, and lists the most commonly occurring amino acid for each residue position in that subgroup to generate a consensus sequence. Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Likewise, nucleic acids encoding antibody chains are aligned when the amino acid sequences encoded by the respective nucleic acids are aligned according to the Kabat numbering convention. An alternative structural definition has been proposed by Chothia, et al., J. Mol. Biol. 196: 901-917, 1987; Chothia, et al., Nature 342: 878-883, 1989; and Chothia, et al., J. Mol. Biol. 186: 651-663, 1989, which are herein incorporated by reference for all purposes.

The nucleic acids of the invention be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (See, e.g., Sambrook, Tijssen and Ausubel discussed herein and incorporated by reference for all purposes). The nucleic acid sequences of the invention and other nucleic acids used to practice this invention, whether RNA, cDNA, genomic DNA, or hybrids thereof, can be isolated from a variety of sources, genetically engineered, amplified, and/or expressed recombinantly. Any recombinant expression system can be used, including, in addition to bacterial, e.g., yeast, insect or mammalian systems. Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, see, e.g., Sambrook, Tijssen and Ausubel. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, □adioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The nucleic acid compositions of the present invention, while often in a native sequence (except for modified restriction sites and the like), from either cDNA, genomic or mixtures can be mutated, thereof in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are contemplated here.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

“Sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

“Detectable” refers to an RNA expression pattern which is detectable via the standard techniques of polymerase chain reaction (PCR), reverse transcriptase-(RT) PCR, differential display, and Northern analyses, which are well known to those of skill in the art. Similarly, protein expression patterns can be “detected” via standard techniques such as Western blots.

As used herein, the phrase “signal transduction pathway” or “signal transduction event” refers to at least one biochemical reaction, but more commonly a series of biochemical reactions, which result from interaction of a cell with a stimulatory compound or agent. Thus, the interaction of a stimulatory compound with a cell generates a “signal” that is transmitted through the signal transduction pathway, ultimately resulting in a cellular response.

A signal transduction pathway refers to the biochemical relationship between a variety of signal transduction molecules that play a role in the transmission of a signal from one portion of a cell to another portion of a cell. As used herein, the phrase “cell surface receptor” includes molecules and complexes of molecules capable of receiving a signal and the transmission of such a signal across the plasma membrane of a cell. An example of a “cell surface receptor” is the T cell receptor (TCR) or the B7 ligands of CTLA-4.

“Activation” as used herein refers to any alteration of a signaling pathway or biological response including, for example, increases above basal levels, restoration to basal levels from an inhibited state, and stimulation of the pathway above basal levels.

A signal transduction pathway in a cell can be initiated by interaction of a cell with a stimulator that is inside or outside of the cell. If an exterior (i.e., outside of the cell) stimulator (e.g., an MHC-antigen complex on an antigen presenting cell) interacts with a cell surface receptor (e.g., a T cell receptor), a signal transduction pathway can transmit a signal across the cell's membrane, through the cytoplasm of the cell, and in some instances into the nucleus. If an interior (e.g., inside the cell) stimulator interacts with an intracellular signal transduction molecule, a signal transduction pathway can result in transmission of a signal through the cell's cytoplasm, and in some instances into the cell's nucleus. An example of a signal transduction pathway is the Tor signal transduction pathway.

Signal transduction can occur through, e.g., the phosphorylation of a molecule; non-covalent allosteric interactions; complexing of molecules; the conformational change of a molecule; calcium release; inositol phosphate production; proteolytic cleavage; cyclic nucleotide production and diacylglyceride production. Typically, signal transduction occurs through phosphorylating a signal transduction molecule.

“Rapamycin” is a bacterial macrolide and a potent immunosuppressant with realized or potential clinical applications in the prevention of graft rejection after organ transplantation and the treatment of autoimmune disorders. This drug acts by forming a complex with the immunophillin FKBP12, and then inhibiting activity of TOR. (Abraham et al., Annu. Rev. Immuno. 14: 483, 1996). Rapamycin treatment of cells has been shown to lead to the dephosphorylation and inactivation of TOR substrates such as P70 S6 Kinase and 4E-BP1/PHAS1. (Dumont et al., J. Immunol 144: 251, 1990; Brown et al., Nature 369: 756, 1994; Kunz et al., Cell 73: 585, 1993; Jefferies et al., EMBO J. 15: 3693, 1997; Beretta et al., EMBO J. 15: 658, 1996). Numerous derivatives of rapamycin are known. Certain 40-O-substituted rapamycins are described in, e.g., in U.S. Pat. No. 5,258,389 and WO 94/09010 (O-alkyl rapamycins); WO 92/05179 (carboxylic acid esters), U.S. Pat. No. 5,118,677 (amide esters), U.S. Pat. No. 5,118,678 (carbamates), U.S. Pat. No. 5,100,883 (fluorinated esters), U.S. Pat. No. 5,151,413 (acetals), and U.S. Pat. No. 5,120,842 (silyl ethers). As described herein, rapamycin (or a rapamycin analog, derivative or related compound thereof) is shown to extend life span by inhibiting the TOR pathway.

This invention relies on routine techniques in the field of recombinant genetics. Basic texts disclosing the general methods of use in this invention include Sambrook et al., Molecular Cloning, A Laboratory Manual, 2″ ed., 1989; Kriegler, Gene Transfer and Expression: A Laboratory Manual, 1990; and Ausubel et al., eds., Current Protocols in Molecular Biology, 1994; all of which are herein incorporated by reference for all purposes.

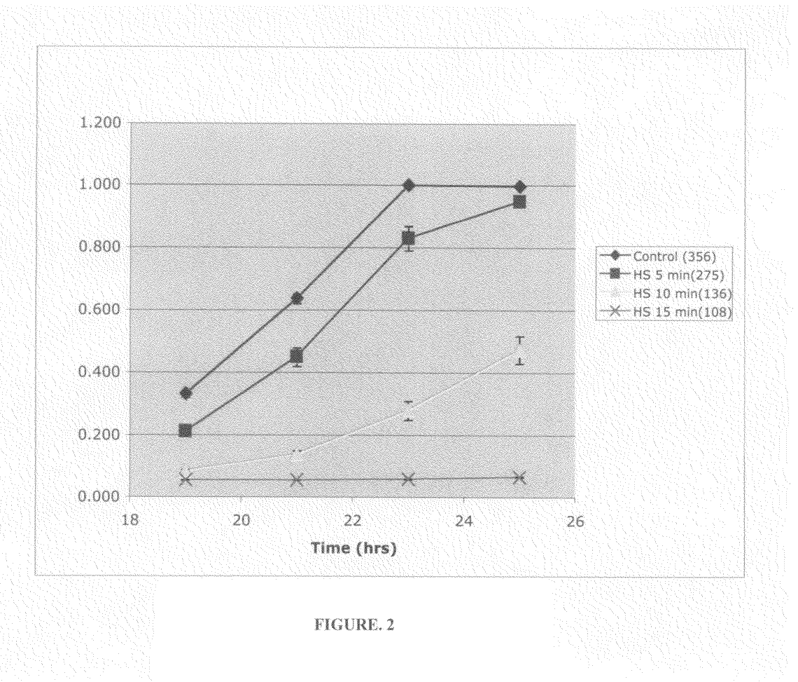

The chronological life span genes, nucleic acids, polymorphic variants, orthologs, and alleles that are substantially identical to sequences provided herein, and referenced by their appropriate GenBank Accession number in FIG. 2, can be isolated using chronological life span nucleic acid probes and oligonucleotides under stringent hybridization conditions, by screening libraries. Alternatively, expression libraries can be used to clone chronological life span protein, polymorphic variants, orthologs, and alleles by detecting expressed homologs immunologically with antisera or purified antibodies made against human chronological life span genes and their gene products or portions thereof.

Aspects of the present invention are directed to (1) high throughput methods for classifying genetic variants, and for identifying “long-lived” variants; (2) methods for identifying genes that regulate life spans (e.g., eukaryotic life spans); (3) various vectors and host cells comprising the identified genes, related orthologs, and related gene products; and (4) pharmaceutical compositions that can modulate, or modify, the function of the identified genes and gene products; and (5) identifying pharmaceutical compositions that have life span extending properties.

### 2. Chronological Life Span Assay—Overview

All of the methods for measuring chronological life span (CLS) described previously require determining viability by plating cells and counting colony-forming units (CFUs), a time consuming and resource limited approach. Methods are provided that will allow for measuring chronological life span in a quantitative, high throughput manner. For example, as provided herein, the quantitative determination of CLS for each single gene deletion mutant present in the Saccharomyces cerevisiae haploid ORF deletion collection (Winzeler et al. 1999 supra) can be performed as well as in other collections.

In order to measure the CLS of yeast cells, it is necessary to (1) maintain a culture of cells in a non-dividing state for at least several weeks and (2) quantitatively measure the viability of cells in the culture over time. CLS assays have been previously performed by the continuous culturing of cells in 5-25 mL of liquid (synthetic complete media, rich media, or water), either in culture tubes on a rotating drum or in flasks on a platform shaker. Each tube or flask contains one strain, and the viability of each strain is measured over time by serial dilution and plating onto rich media for determination of CFUs. These methods work well for a relatively small number of strains (<20) assayed in parallel; however, it is not feasible to perform genome-wide analysis of more than 4000 strains in this manner. Therefore, as described herein, technology is disclosed that is more suitable for the simultaneous quantitative measurement of CLS for several thousand strains. Several major improvements over the traditional CLS assays have been made, including (1) measurement of viability (relative to a reference) by optical density (OD), (2) long-term culturing of cells in sub-milliliter volumes using 96-well microtiter plates, and (3) utilization of robotic systems for automated dilution and cell transfer.

Previous methods for determination of CLS have relied upon culturing each yeast strain in 5-25 mL of liquid, removing aliquots every 1-3 days, and measuring the fraction of viable cells in the culture by serial dilution, plating, and counting CFUs. Determination of viability using the determination of viability by OD after outgrowth (DVOD) (rather than CFU counting) allows for the simultaneous measurement of viability for up to several thousand cultures; however, maintaining this many cultures in 5-25 mL volumes is impractical. Therefore, the assay and methods disclosed herein allow the entire CLS assay to be performed in 96-well microtiter plates. This technology has several practical benefits, including (1) consumption of fewer resources (media, Petri dishes, culture tubes, and the like) and lab space, (2) ease of automation using robotic equipment to transfer cells and dispense media and cells, and (3) allowing high throughput screening of genetic or environmental (e.g., chemical) parameters that affect chronological aging. A commonly used assays for measuring CLS (e.g., the chronological life span synthetic complete (CLSSC) assay) is amenable to this type of approach. CLSSC refers to It refers to the method of measuring chronological life span when the cells are cultured in synthetic complete media.

### 3. Chronological Life Span Assay—Methodology

A schematic diagram for a high throughput chronological life span analysis of the present invention is shown in FIG. 1. A cell culture plate is inoculated with cells and incubated throughout the experiment (A). Each well of the plate can contain cells treated with different drugs, with genetic modifications, or other experimental manipulations. At multiple time intervals, small, equal volumes of cells are transferred from the cell culture plate into a corresponding well of a second plate containing fresh media. This second plate is then incubated and the optical density (OD) of each well is measured after incubation. The OD of each well after this out-growth period is highly correlated to the number of viable cells that originally were transferred to that well from the aging plate, which allows for determination of the percentage of cells that remain viable in each well of the aging plate. This high throughput CLS method can be applied to wide range of assays commonly used by one of skill in the art such as the CLSSC assay.

The high throughput CLSSC assay involves culturing yeast cells in 96-well microtiter plates, where each well contains one strain (or chemical) and 100-200 μL of synthetic complete media, which is a standard, chemically defined media used for culturing yeast cells. Viability of each strain is determined periodically using the DVOD method by transferring 2 μl, from each well of the plate containing the aging cells into the corresponding well of a new microtiter plate containing 200 μL of growth media. Transfer is accomplished in an automated fashion using a Biomek FX with 96-pin HDR tool. After an appropriate incubation period, OD₆₆₀ is determined for each well using a plate reader (e.g., a Victor plate reader). The average OD₆₆₀ for each well on day zero is defined as 100% viability, and the average OD₆₆₀ for each well on subsequent days is used to calculate percent viability over time.

This high throughput CLSSC method was used in a qualitative screen for deletion mutations that dramatically extend maximum CLS (see below).

The disclosed methods allow high throughput determination of CLS by various methods known to one of skill in the art, or by variations of these approaches. For example, the high throughput CLSSC assay is performed by culturing each strain in synthetic complete (SC) in 96-well microtiter plates. Viability is determined periodically (every 3-7 days) for both methods using the DVOD method, as described above. The average OD₆₆₀ for each well on day zero is defined as 100% viability, and the average OD₆₆₀ for each well on subsequent days is used to calculate percent viability over time.

### 4. High Throughput Format

An assay performed in a “homogeneous format” means that the assay can be performed in a single container, with no manipulation or purification of any components being required to determine the result of the assay, e.g., a test agent can be added to an assay system and any effects directly measured. Often, such “homogeneous format” assays will comprise at least one component that is “quenched” or otherwise modified in the presence or absence of a test agent.

A “secondary screening step” refers to a screening step whereby a test agent is assessed for a secondary property in order to determine the specificity or mode of action of a compound identified using the methods provided herein. Such secondary screening steps can be performed on all of the test agents, or, e.g., on only those that are found to be positive in a primary screening step, and can be performed subsequently, simultaneously, or prior to a primary screening step.

“High throughput screening” refers to a method of rapidly assessing a large number of test agents for a specific activity. Typically, the plurality of test agents will be assessed in parallel, for example by simultaneously assessing 96 or 384 agents using a 96-well or 384-well plate, 96-well or 384-well dispensers, and detection methods capable of detecting 96 or 384 samples simultaneously. Often, such methods will be automated, e.g., using robotics.

“Robotic high throughput screening” refers to high throughput screening that involves at least one robotic element, thereby eliminating a requirement for human manipulation in at least one step of the screening process. For example, a robotic arm can dispense a plurality of test agents to a multi-well plate.

A “multi-well plate” refers to any container, receptacle, or device that can hold a plurality of samples, e.g., for use in high throughput screening. Typically, such “multi-well plates” will be part of an integrated and preferably automated system that enables the rapid and efficient screening or manipulation of a large number of samples. Such plates can include, e.g., 24, 48, 96, 384, or more wells, and are typically used in conjunction with a 24, 48, 96, 384, or more tip pipettors, samplers, detectors, and the like.

In some assays, it will be desirable to have positive controls to ensure that the components of the assays are working properly.

In the high throughput assays of the invention, it is possible to screen up to several thousand different modulators in a single day. In particular, each well of a microtiter plate can be used to run a separate assay against a selected potential modulator, or if concentration or incubation time effects are to be observed, every 5-10 wells can test a single modulator. Thus, a single standard microtiter plate can assay about 100 (96) modulators. If 1536 well plates are used, then a single plate can easily assay from about 100 to about 1500 different compounds. It is possible to assay many different plates per day; assay screens for up to about 6,000-20,000, and even up to about 100,000-1,000,000 different compounds are possible using the integrated systems of the invention.

### 5. Solid State and Soluble High Throughput Assays

The invention provides soluble assays using a chronological life span gene or gene product, or a cell or tissue expressing a chronological life span gene product, either naturally occurring or recombinant. The invention further provide solid phase based in vitro assays in a high throughput format, where a chronological life span protein or fragment thereof, is attached to a solid phase substrate.

In the high throughput assays of the invention, either soluble or solid state, it is possible to screen up to several thousand different modulators or ligands in a single day. This methodology can be used for the chronological life span proteins in vitro, or for cell-based or membrane-based assays comprising a chronological life span protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day; assay screens for up to about 6,000, 20,000, 50,000, or more than 100,000 different compounds are possible using the integrated systems of the invention. For a solid state reaction, the protein of interest or a fragment thereof, e.g., an extracellular domain, or a cell or membrane comprising the chronological life span protein of interest or a fragment thereof as part of a fusion protein can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, and the like) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, and the like), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1963 (describing solid phase synthesis of, e.g., peptides); Geysen et al., J. Immun. Meth. 102: 259-274, 1987 (describing synthesis of solid phase components on pins); Frank & Doring, Tetrahedron 44:60316040 (1988) (describing synthesis of various peptide sequences on cellulose disks); Fodor et al., Science, 251: 767-777, 1991; Sheldon et al., Clinical Chemistry 39(4): 718-719, 1993; and Kozal et al., Nature Medicine 2(7):753-759, 1996 (all describing arrays of biopolymers fixed to solid substrates). Non-chemical approaches for fixing tag binders to substrates include other common methods, such as heat, cross-linking by UV radiation, and the like.

### 6. Yeast Deletion Mutant Collection

For the analysis of yeast variants, examples of suitable libraries include various yeast haploid deletion collections, such as ORF-deletion collections made in various suitable backgrounds. For example, a Saccharomyces. cerevisiae Genome Deletion and Parallel collection (SCGDP) contains an almost complete set of genetic variants in which a single-ORF is replaced with a KanMX selectable marker. Several isogenic, S288C-derived, designer deletion variants containing different genetic backgrounds for SCGDP exists, including the BY4741 (MATa), BY4742 (MATO, and BY4743 (MATa/MATα) strains. (Winzeler et al., Science 285: 901-906, 1999). Four deletion collections (haploid MATa, haploid MATα heterozygous diploid, and homozygous diploid) representing greater than 6000 unique gene disruptions can be employed for the identification of any subset exhibiting a phenotype of interest, including a long life span.

Variants for analysis by methods of the present invention include naturally occurring variants that arise spontaneously in a laboratory and in nature, and genetic variants that are generated in a laboratory using various mutation-inducing methods known to persons skilled in the art. Variants can be generated in any gene, by various methods, including chemical mutagenesis induced by exposure to a mutagen, such as ethane methyl sulfonate (EMS), radiation-induced mutagenesis, and various genetic-engineering techniques, such as PCR-mediated mutations, transposon mutagenesis, site-directed mutagenesis, or gene over-expression techniques. Suitable mutations include point mutations, gene deletions, gene insertions, and any modification of genomic sequences that results in a change in gene expression, such as the over-expression, modification, or inactivation of at least one gene or gene product. Contemplated variants include various species of plants; invertebrates, such as yeasts, insects (e.g., Drosphila, and worms; and vertebrates, such as mammals or mammalian cells).

### 7. Chronological Life Span Regulating Genes and Functionally-Related Orthologs

Genes that confer longevity within identified chronological life span (CLS) variants can be functionally retested to determine whether the longevity effect observed in CLS variants is reproducible. For example, new deletion strains can be re-created by standard homologous recombination methods, and CLS for re-created deletion strains can be determined. If a deletion of the gene in question results in life spans substantially higher than that of a “wildtype” reference, then the deleted gene is correctly identified. If the re-created deletion strain is determined to exhibit a life span similar to that of a “wildtype” reference, then it is possible that a genetic change unrelated to a particular known deletion mutation may cause a “long-lived” phenotype observed initially in a variant classified as a CLS variant. Various methods known to persons skilled in the art can be utilized to confirm bona fide genes that regulate life spans, and exclude genes that are not related to life-span regulation but are falsely detected.

### 8. General Techniques

The nucleic acids used to practice this invention, whether RNA, iRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams, J. Am. Chem. Soc. 105: 661, 1983; Belousov, Nucleic Acids Res. 25: 3440-3444, 1997; Frenkel, Free Radic. Biol. Med. 19: 373-380, 1995; Blommers, Biochemistry 33: 7886-7896, 1994; Narang, Meth. Enzymol. 68: 90, 1979; Brown Meth. Enzymol. 68: 109, 1979; Beaucage, Tetra. Lett. 22: 1859, 1981; U.S. Pat. No. 4,458,066.

The invention provides oligonucleotides comprising sequences of the invention, e.g., subsequences of the exemplary sequences of the invention. Oligonucleotides can include, e.g., single stranded poly-deoxynucleotides or two complementary polydeoxynucleotide strands which can be chemically synthesized.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2^{ND} ED.), Vols. 1-3, Cold Spring Harbor Laboratory, 1989; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y., 1993.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g., fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld, Nat. Genet. 15: 333-335, 1997; yeast artificial chromosomes (YAC); bacterial artificial chromosomes (BAC); P1 artificial chromosomes, see, e.g., Woon, Genomics 50: 306-316, 1998; P1-derived vectors (PACs), see, e.g., Kern, Biotechniques 23: 120-124, 1997; cosmids, recombinant viruses, phages or plasmids.

The invention provides fusion proteins and nucleic acids encoding them. A chronological life span polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego, Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site. (See e.g., Williams, Biochemistry 34: 1787-1797, 1995; Dobeli, Protein Expr. Purif 12: 404-414, 1998). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll, DNA Cell Biol. 12: 441-53, 1993.

### 9. Transcriptional Control Elements

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

### 10. Expression Vectors and Cloning Vehicles

The invention provides expression vectors and cloning vehicles comprising nucleic acids of the invention, e.g., sequences encoding the proteins of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The invention provides libraries of expression vectors encoding polypeptides and peptides of the invention. These nucleic acids can be introduced into a genome or into the cytoplasm or a nucleus of a cell and expressed by a variety of conventional techniques, well described in the scientific and patent literature. See, e.g., Roberts, Nature 328: 731, 1987; Schneider, Protein Expr. Purif. 6435: 10, 1995; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors can also be employed which exploit advantageous merits of each of the parent vector properties. (See e.g., Feng, Nature Biotechnology 15: 866-870, 1997). Such viral genomes can be modified by recombinant DNA techniques to include the nucleic acids of the invention; and can be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher, J. Virol. 66: 2731-2739, 1992; Johann, J. Virol. 66: 1635-1640, 1992). Adeno-associated virus (AAV)-based vectors can be used to □adioimmun cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada, Gene Ther. 3: 957-964, 1996.

“Expression cassette” as used herein refers to a nucleotide sequence which is capable of affecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression can also be used, e.g., enhancers.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination. Thus, expression cassettes also include plasmids, expression vectors, recombinant viruses, any form of recombinant “naked DNA” vector, and the like.

“Vector” is intended to refer to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### 11. Host Cells and Transformed Cells

The invention also provides a transformed cell comprising a nucleic acid sequence of the invention, e.g., a sequence encoding a polypeptide of the invention, or a vector of the invention. The host cell can be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter can be induced by appropriate means (e.g., temperature shift or chemical induction) and the cells can be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct can be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

### 12. Amplification of Nucleic Acids

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides of the invention, e.g., primer pairs capable of amplifying nucleic acid sequences comprising the exemplary sequences in FIG. 1, or subsequences thereof.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4: 560, 1989; Landegren, Science 241: 1077, 1988; Barringer, Gene 89: 117, 1990); transcription amplification (see, e.g., Kwoh, Proc. Natl. Acad. Sci. USA 86: 1173, 1989); and, self-sustained sequence replication (see, e.g., Guatelli, Proc. Natl. Acad. Sci. USA 87: 1874, 1990); Q Beta replicase amplification (see, e.g., Smith, J. Clin. Microbiol. 35: 1477-1491, 1997), automated Q-beta replicase amplification assay (see, e.g., Burg, Mol. Cell. Probes 10: 257-271, 1996) and other RNA polymerase mediated techniques (e.g., NASBA, Cangene, Mississauga, Ontario); see also Berger, Methods Enzymol. 152: 307-316, 1987; Sambrook; Ausubel; U.S. Pat. Nos. 4,683,195 and 4,683,202; Sooknanan, Biotechnology 13: 563-564, 1995.

### 13. Hybridization of Nucleic Acids

The invention provides isolated or recombinant nucleic acids that hybridize under stringent conditions to an exemplary sequence of the invention, e.g., a sequence represented by the indicated genes in Table 1 or in Table 2, or the complement of any thereof, or a nucleic acid that encodes a polypeptide of the invention. In alternative aspects, the stringent conditions are highly stringent conditions, medium stringent conditions or low stringent conditions, as known in the art and as described herein. These methods can be used to isolate nucleic acids of the invention.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

“Selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one embodiment, a nucleic acid can be determined to be within the scope of the invention by its ability to hybridize under stringent conditions to a nucleic acid otherwise determined to be within the scope of the invention (such as the exemplary sequences described herein).

“Stringent hybridization conditions” refers to conditions under which a probe will hybridize to its target subsequence, typically in a complex mixture of nucleic acid, but not to other sequences in significant amounts (a positive signal (e.g., identification of a nucleic acid of the invention) is about 10 times background hybridization). Stringent conditions are sequence-dependent and will be different in different circumstances. Longer sequences hybridize specifically at higher temperatures. An extensive guide to the hybridization of nucleic acids is found in e.g., Sambrook, ed., Molecular Cloning: A Laboratory Manual (2^{nd} Ed.), Vols. 1-3, Cold Spring Harbor Laboratory, 1989; Current Protocols in Molecular Biology, Ausubel, ed. John Wiley & Sons, Inc., New York, 1997; Laboratory Techniques In Biochemistry And Molecular Biology Hybridization With Nucleic Acid Probes, Part I. Theory and Nucleic Acid Preparation, Tijssen, ed. Elsevier, N.Y., 1993.

Generally, stringent conditions are selected to be about 5-10° C. lower than the thermal melting point I for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide as described in Sambrook (cited below). For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. For selective or specific hybridization, a positive signal (e.g., identification of a nucleic acid of the invention) is about 10 times background hybridization. Stringent hybridization conditions that are used to identify nucleic acids within the scope of the invention include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. In the present invention, genomic DNA or cDNA comprising nucleic acids of the invention can be identified in standard Southern blots under stringent conditions using the nucleic acid sequences disclosed here. Additional stringent conditions for such hybridizations (to identify nucleic acids within the scope of the invention) are those which include a hybridization in a buffer of 40% formamide, 1 M NaCl, 1% SDS at 37° C.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

### 14. Oligonucleotides Probes and Methods for Using them

The invention also provides nucleic acid probes for identifying nucleic acids encoding a polypeptide which is a modulator of a chronological life span-signaling activity. In one aspect, the probe comprises at least 10 consecutive bases of a nucleic acid of the invention. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a sequence as set forth in a nucleic acid of the invention. The probes identify a nucleic acid by binding and/or hybridization. The probes can be used in arrays of the invention, see discussion below. The probes of the invention can also be used to isolate other nucleic acids or polypeptides.

### 15. Determining the Degree of Sequence Identity

The invention provides nucleic acids having at least 90%, 91%, 92%, 93%, 94%, 95%, 96%, 97%, 98%, 99% or more sequence identity to the sequences of the present invention as shown in FIG. 1. The invention provides polypeptides having at least 90%, 91%, 92%, 93%, 94%, 95%, 96%, 97%, 98%, 99% or more sequence identity sequences of the present invention as shown in FIG. 1. The sequence identities can be determined by analysis with a sequence comparison algorithm or by a visual inspection. Protein and/or nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Natl. Acad. Sci. U.S.A. 85: 2444, 1988, by computerized implementations of these algorithms (FASTDB (Intelligenetics), BLAST (National Center for Biomedical Information), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., (1999 Suppl.), Current Protocols in Molecular Biology, Greene Publishing Associates and Wiley Interscience, N.Y., 1987)

A preferred example of an algorithm that is suitable for determining percent sequence identity and sequence similarity is the FASTA algorithm, which is described in Pearson & Lipman, Proc. Natl. Acad. Sci. U.S.A. 85: 2444, 1988. See also Pearson, Methods Enzymol. 266: 227-258, 1996. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another preferred example of algorithm that is suitable for determining percent sequence identity and sequence similarity are the BLAST and BLAST 2.0 algorithms, which are described in Altschul et al., Nuc. Acids Res. 25: 3389-3402, 1977; and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89: 10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90: 5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35: 351-360, 1987. The method used is similar to the method described by Higgins & Sharp, CABIOS 5: 151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0. (Devereaux et al., Nuc. Acids Res. 12: 387-395, 1984).

Another preferred example of an algorithm that is suitable for multiple DNA and amino acid sequence alignments is the CLUSTALW program. (Thompson et al., Nucl. Acids. Res. 22: 4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix. (Henikoff and Henikoff, Proc. Natl. Acad. Sci. U.S.A. 89: 10915-10919, 1992).

“Sequence identity” refers to a measure of similarity between amino acid or nucleotide sequences, and can be measured using methods known in the art, such as those described below:

“Identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

“Substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the following sequence comparison algorithms or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred embodiment, the sequences are substantially identical over the entire length of the coding regions.

“Homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (an exemplary sequence of the present invention for any of the genes listed in Table 1) to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, continugous residues ranging anywhere from 20 to the full length of an exemplary polypeptide or nucleic acid sequence of the invention, are compared to a reference sequence of the same number of contiguous positions after the two sequences are optimally aligned. If the reference sequence has the requisite sequence identity to an exemplary polypeptide or nucleic acid sequence of the invention, e.g., at least 90%, 91%, 92%, 93%, 94%, 95%, 96%, 97%, 98%, 99% or more sequence identity to the sequences of the invention (see FIG. 1 and the Examples), that sequence is within the scope of the invention.

Motifs which can be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

### 16. Database Searching/Sequence Alignments, Computer Systems, Computer Program Products and Databases

Genomic databases for model organisms of various species can be employed for conducting multi-genome-wide sequence alignments in order to identify homologous sequences of interest. For each identified yeast sequence (e.g., for those sequences of the genes listed in Table 1), related orthologous sequences can be determined by searching composite genomic databases (see the Table 2 for a listing of identified sequences, including various exemplary mammalian orthologs). The breath of a database search is limited by the scope of representative model organisms for which sequence data is available.

Homology can be determined by various methods, including alignments of open-reading-frames (“ORFs”) contained in private and/or public databases. Any suitable mathematical algorithm may be used to determine percent identities and percent similarities between any two sequences being compared. For example, nucleic acid and protein sequences of the present invention can be used as a “query sequence” to perform a search against sequences deposited within various public databases to identify other family members or evolutionarily-related sequences. Genomic sequences for various organisms are currently available, including fungi, such as the budding yeast, or Saccharomyces cerevisiae; invertebrates, such as Caenorhabditis elegans and Drosophila melangaster; and mammals, such as the mouse, rat, and human. Exemplary databases for identifying orthologs of interest include Genebank, Swiss Protein, EMBL, and National Center for Biotechnology Information (“NCBI”), and many others known in the art. These databases enable a user to set various parameters for a hypothetical search according to the user's preference, or to utilize default settings. As discussed above, the Examples provide listing of identified sequences, including various exemplary mammalian orthologs of the invention.

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media can be a hard disk, a floppy disk, a magnetic tape, CD-ROM, Digital Versatile Disk (DVD), Random Access Memory (RAM), or Read Only Memory (ROM) as well as other types of other media known to those skilled in the art.

As used herein, the terms “computer,” “computer program” and “processor” are used in their broadest general contexts and incorporate all such devices.

### 17. Candidate Bioactive Agents

Having identified a number of chronological life span genes and their homologs (see, e.g., Table 1 and the listing in Table 2), the information can be used in a wide variety of ways. In a preferred method, the genes can be used in conjunction with high throughput screening techniques as described herein, to allow monitoring for genes after treatment with a candidate agent, Zlokarnik et al., Science 279: 84-8, 1998; Heid et al., Genome Res. 6: 986, 1996. In a preferred method, the candidate agents are added to cells.

The term “modulator”, “candidate substance”, “candidate bioactive agent”, “drug candidate”, “agent” or grammatical equivalents as used herein describes any molecule, e.g., protein, oligopeptide, small organic molecule, polysaccharide, polynucleotide, to be tested for bioactive agents that are capable of directly or indirectly altering the activity of a target gene, protein, or cell. In preferred methods, the bioactive agents modulate the expression profiles, or expression profile nucleic acids or proteins provided herein. In a particularly preferred method, the candidate agents induce a response, or maintain such a response as indicated, for example, by the effect of the agent on the expression profile, nucleic acids, proteins or activity as further described below. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs.

In some preferred embodiments, the candidate bioactive agents are proteins. By “protein” herein is meant at least two covalently attached amino acids, which includes proteins, polypeptides, oligopeptides and peptides. The protein can be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains can be in either the (R) or the (S) configuration. In some preferred embodiment, the amino acids are in the (S) or L-configuration. If non-naturally occurring side chains are used, non-amino acid substituents can be used, for example to prevent or retard in vivo degradations.

In a preferred method, the candidate bioactive agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening in the methods of the invention. The libraries can be bacterial, fungal, viral, and mammalian proteins, with the latter being preferred, and human proteins.

In some methods, the candidate bioactive agents are peptides of from about 5 to about 30 amino acids, with from about 5 to about 20 amino acids being preferred, and from about 7 to about 15 being particularly preferred. The peptides can be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they can incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In some methods, the library can be fully randomized, with no sequence preferences or constants at any position. In other methods, the library can be biased. Some positions within the sequence are either held constant, or are selected from a limited number of possibilities. For example, in some methods, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, or to purines. In other methods, the candidate bioactive agents are nucleic acids, as defined above.

As described above generally for proteins, nucleic acid candidate bioactive agents can be naturally occurring nucleic acids, random nucleic acids, or “biased” random nucleic acids. For example, digests of procaryotic or eucaryotic genomes can be used as is outlined above for proteins.

In some methods, the candidate bioactive agents are organic chemical moieties.

### 18. Inhibiting Expression of Polypeptides and Transcripts

The invention further provides for nucleic acids complementary to (e.g., antisense sequences to) the nucleic acid sequences of the invention. Antisense sequences are capable of inhibiting the transport, splicing or transcription of protein-encoding genes, e.g., the chronological life span polypeptides encoding nucleic acids of the invention. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides which cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention. The term “RNAi” stands for RNA interference. This term is understood in the art to encompass technology using RNA molecules that can silence genes. (See, for example, McManus, et al., Nature Reviews Genetics 3: 737, 2002). In this application, the term “RNAi” encompasses molecules such as short interfering RNA (siRNA), microRNAs (mRNA), small temporal RNA (stRNA). Generally speaking, RNA interference results from the interaction of double-stranded RNA with genes.

### 19. Antisense Oligonucleotides

The invention provides antisense oligonucleotides capable of binding the chronological life span polypeptide message which can inhibit polypeptide activity by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata, Toxicol Appl Pharmacol 144: 189-197, 1997; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

The invention provides a method of inhibiting expression of a gene encoding a chronological life span protein comprising the step of (i) providing a biological system in which expression of a gene encoding a chronological life span protein is to be inhibited; and (ii) contacting the system with an antisense molecule that hybridizes to a transcript encoding the chronological life span protein. In other embodiments, chronological life span proteins are inhibited. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression may be inducible and/or tissue or cell type-specific. The antisense molecule may be an oligonucleotide or a longer nucleic acid molecule. The invention provides such antisense molecules.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention. (See, e.g., Gold, J. Biol. Chem. 270: 13581-13584, 1995).

### 20. siRNA

RNA interference (RNAi) is a mechanism of post-transcriptional gene silencing mediated by double-stranded RNA (dsRNA), which is distinct from antisense and ribozyme-based approaches. (see Jain, Pharmacogenomics 5: 239-42, 2004 for a review of RNAi and siRNA). RNA interference is useful in a method for treating a chronological life span disease state or disease or disorder related to aging in a mammal by administering to the mammal a nucleic acid molecule (e.g., dsRNA) that hybridizes under stringent conditions to a chronological life span, and attenuates expression of said target gene. dsRNA molecules are believed to direct sequence-specific degradation of mRNA in cells of various types after first undergoing processing by an RNase III-like enzyme called DICER (Bernstein et al., Nature 409: 363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

In general, a short, interfering RNA (siRNA) comprises an RNA duplex that is preferably approximately 19 basepairs long and optionally further comprises one or two single-stranded overhangs or loops. An siRNA may comprise two RNA strands hybridized together, or may alternatively comprise a single RNA strand that includes a self-hybridizing portion. siRNAs may include one or more free strand ends, which may include phosphate and/or hydroxyl groups. siRNAs typically include a portion that hybridizes under stringent conditions with a target transcript. One strand of the siRNA (or, the self-hybridizing portion of the siRNA) is typically precisely complementary with a region of the target transcript, meaning that the siRNA hybridizes to the target transcript without a single mismatch. In certain embodiments of the invention in which perfect complementarity is not achieved, it is generally preferred that any mismatches be located at or near the siRNA termini.

siRNAs have been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, Nat. Biotechnol. 20: 446-448, 2002; See also Yu et al., Proc. Natl. Acad. Sci., 99: 6047-6052, 2002; Sui et al., Proc. Natl. Acad. Sci USA., 99: 5515-5520, 2002; Paddison et al., Genes and Dev. 16: 948-958, 2002; Brummelkamp et al., Science 296: 550-553, 2002; Miyagashi and Taira, Nat. Biotech. 20: 497-500, 2002; Paul et al., Nat. Biotech. 20: 505-508, 2002. As described in these and other references, the siRNA can consist of two individual nucleic acid strands or of a single strand with a self-complementary region capable of forming a hairpin (stem-loop) structure. A number of variations in structure, length, number of mismatches, size of loop, identity of nucleotides in overhangs, and the like, are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is preferred to target exons rather than introns, and it can also be preferable to select sequences complementary to regions within the 3′ portion of the target transcript. Generally it is preferred to select sequences that contain approximately equimolar ratio of the different nucleotides and to avoid stretches in which a single residue is repeated multiple times.

siRNAs can thus comprise RNA molecules having a double-stranded region approximately 19 nucleotides in length with 1-2 nucleotide 3′ overhangs on each strand, resulting in a total length of between approximately 21 and 23 nucleotides. As used herein, siRNAs also include various RNA structures that can be processed in vivo to generate such molecules. Such structures include RNA strands containing two complementary elements that hybridize to one another to form a stem, a loop, and optionally an overhang, preferably a 3′ overhang. Preferably, the stem is approximately 19 by long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and can be up to approximately 29 nucleotides in length. Loops of 4 nucleotides or greater are less likely subject to steric constraints than are shorter loops and therefore can be preferred. The overhang can include a 5′ phosphate and a 3′ hydroxyl. The overhang can but need not comprise a plurality of U residues, e.g., between 1 and 5 U residues. Classical siRNAs as described above trigger degradation of mRNAs to which they are targeted, thereby also reducing the rate of protein synthesis. In addition to siRNAs that act via the classical pathway, certain siRNAs that bind to the 3′ UTR of a template transcript can inhibit expression of a protein encoded by the template transcript by a mechanism related to but distinct from classic RNA interference, e.g., by reducing translation of the transcript rather than decreasing its stability. Such RNAs are referred to as microRNAs (mRNAs) and are typically between approximately 20 and 26 nucleotides in length, e.g., 22 nt in length. It is believed that they are derived from larger precursors known as small temporal RNAs (stRNAs) or mRNA precursors, which are typically approximately 70 nt long with an approximately 4-15 nt loop. (See Grishok et al., Cell 106: 23-24, 2001; Hutvagner et al., Science 293: 834-838, 2001; Ketting, et al., Genes Dev., 15: 2654-2659, 2001). Endogenous RNAs of this type have been identified in a number of organisms including mammals, suggesting that this mechanism of post-transcriptional gene silencing can be widespread. (Lagos-Quintana et al., Science 294: 853-858, 2001; Pasquinelli, Trends in Genetics 18: 171-173, 2002, and references in the foregoing two articles). MicroRNAs have been shown to block translation of target transcripts containing target sites in mammalian cells. (Zeng et al., Molecular Cell 9: 1-20, 2002).

siRNAs such as naturally occurring or artificial (i.e., designed by humans) mRNAs that bind within the 3′ UTR (or elsewhere in a target transcript) and inhibit translation can tolerate a larger number of mismatches in the siRNA/template duplex, and particularly can tolerate mismatches within the central region of the duplex. In fact, there is evidence that some mismatches can be desirable or required as naturally occurring stRNAs frequently exhibit such mismatches as do mRNAs that have been shown to inhibit translation in vitro. For example, when hybridized with the target transcript such siRNAs frequently include two stretches of perfect complementarity separated by a region of mismatch. A variety of structures are possible. For example, the mRNA can include multiple areas of nonidentity (mismatch). The areas of nonidentity (mismatch) need not be symmetrical in the sense that both the target and the mRNA include nonpaired nucleotides. Typically the stretches of perfect complementarity are at least 5 nucleotides in length, e.g., 6, 7, or more nucleotides in length, while the regions of mismatch can be, for example, 1, 2, 3, or 4 nucleotides in length.

Hairpin structures designed to mimic siRNAs and mRNA precursors are processed intracellularly into molecules capable of reducing or inhibiting expression of target transcripts. (McManus et al., RNA 8: 842-850, 2002). These hairpin structures, which are based on classical siRNAs consisting of two RNA strands forming a 19 by duplex structure are classified as class I or class II hairpins. Class I hairpins incorporate a loop at the 5′ or 3′ end of the antisense siRNA strand (i.e., the strand complementary to the target transcript whose inhibition is desired) but are otherwise identical to classical siRNAs. Class II hairpins resemble mRNA precursors in that they include a 19 nt duplex region and a loop at either the 3′ or 5′ end of the antisense strand of the duplex in addition to one or more nucleotide mismatches in the stem. These molecules are processed intracellularly into small RNA duplex structures capable of mediating silencing. They appear to exert their effects through degradation of the target mRNA rather than through translational repression as is thought to be the case for naturally occurring mRNAs and stRNAs.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an siRNA, and any structure that generates such an siRNA (i.e., serves as a precursor to the RNA) is useful in the practice of the present invention.

In the context of the present invention, siRNAs are useful both for therapeutic purposes, e.g., to modulate the expression of a chronological life span molecule or protein in a subject at risk of or suffering from a chronological life span disease or disorder or disease related to aging including various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease and for the inventive methods for the identification of compounds for treatment of a chronological life span molecule or protein in a subject at risk of or suffering from a chronological life span disease or disorder or disease related to aging including various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease that modulate the activity or level of the molecules described herein. In one aspect, the molecules can encode, interact with or be a gene product associated with any of the genes listed in Table 1 or in Table 2. In another aspect, the therapeutic treatment of chronological life span disease target with an antibody, antisense vector, or double stranded RNA vector.

The invention therefore provides a method of inhibiting expression of a gene encoding a chronological life span protein comprising the step of (i) providing a biological system in which expression of a gene encoding chronological life span protein is to be inhibited; and (ii) contacting the system with an siRNA targeted to a transcript encoding the chronological life span protein. In other embodiments, chronological life span proteins are inhibited. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

By “biological system” is meant any vessel, well, or container in which biomolecules (e.g., nucleic acids, polypeptides, polysaccharides, lipids, and the like) are placed; a cell or population of cells; a tissue; an organ; an organism, and the like. Typically the biological system is a cell or population of cells, but the method can also be performed in a vessel using purified or recombinant proteins.

The invention provides siRNA molecules targeted to a transcript encoding any chronological life span protein or chronological life span-related protein. In particular, the invention provides siRNA molecules selectively or specifically targeted to a transcript encoding a polymorphic variant of such a transcript, wherein existence of the polymorphic variant in a subject is indicative of susceptibility to or presence of a chronological life span-related disease or disease or disorder associated with aging. The terms “selectively” or “specifically targeted to”, in this context, are intended to indicate that the siRNA causes greater reduction in expression of the variant than of other variants (i.e., variants whose existence in a subject is not indicative of susceptibility to or presence of a chronological life span disease, disorder or related disease or disorder). The siRNA, or collections of siRNAs, can be provided in the form of kits with additional components as appropriate.

### 21. Short Hairpin RNA (shRNA)

RNA interference (RNAi), a mechanism of post-transcriptional gene silencing mediated by double-stranded RNA (dsRNA), is useful in a method for treating a chronological life span disease state or disease state related to aging in a mammal by administering to the mammal a nucleic acid molecule (e.g., dsRNA) that hybridizes under stringent conditions to a chronological life span gene, and attenuates expression of said target gene. See Jain, Pharmacogenomics 5: 239-42, 2004 for a review of RNAi and siRNA. A further method of RNA interference in the present invention is the use of short hairpin RNAs (shRNA). A plasmid containing a DNA sequence encoding for a particular desired siRNA sequence is delivered into a target cell via transfection or virally-mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression. Inhibition of protein expression by transfected siRNAs is a transient phenomenon that does not occur for times periods longer than several days. In some cases, this can be preferable and desired. In cases where longer periods of protein inhibition are necessary, shRNA mediated inhibition is preferable.

### 22. Full and Partial Length Antisense RNA Transcripts

Antisense RNA transcripts have a base sequence complementary to part or all of any other RNA transcript in the same cell. Such transcripts have been shown to modulate gene expression through a variety of mechanisms including the modulation of RNA splicing, the modulation of RNA transport and the modulation of the translation of mRNA. (Denhardt, Ann N Y Acad. Sci. 660: 70, 1992; Nellen, Trends Biochem. Sci. 18: 419, 1993; Baker and Monia, Biochem. Biophys. Acta, 1489: 3, 1999; Xu et al., Gene Therapy 7: 438, 2000; French and Gerdes, Curr. Opin. Microbiol. 3: 159, 2000; Terryn and Rouze, Trends Plant Sci. 5: 1360, 2000).

### 23. Antisense RNA and DNA Oligonucleotides

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but can range from 10 up to approximately 50 nucleotides in length. Binding typically reduces or inhibits the function of the target nucleic acid. For example, antisense oligonucleotides can block transcription when bound to genomic DNA, inhibit translation when bound to mRNA, and/or lead to degradation of the nucleic acid. Reduction in expression of a chronological life span or chronological life span polypeptide can be achieved by the administration of antisense nucleic acids or peptide nucleic acids comprising sequences complementary to those of the mRNA that encodes the polypeptide. Antisense technology and its applications are well known in the art and are described in Phillips, M. I. (ed.) Antisense Technology, Methods Enzymol., 2000, Volumes 313 and 314, Academic Press, San Diego, and references mentioned therein. See also Crooke, S. (ed.) “Antisense Drug Technology: Principles, Strategies, and Applications” (1^{st} Edition) Marcel Dekker; and references cited therein.

Antisense oligonucleotides can be synthesized with a base sequence that is complementary to a portion of any RNA transcript in the cell. Antisense oligonucleotides can modulate gene expression through a variety of mechanisms including the modulation of RNA splicing, the modulation of RNA transport and the modulation of the translation of mRNA. (Denhardt, Ann N Y Acad. Sci. 660: 70, 1992). Various properties of antisense oligonucleotides including stability, toxicity, tissue distribution, and cellular uptake and binding affinity can be altered through chemical modifications including (i) replacement of the phosphodiester backbone (e.g., peptide nucleic acid, phosphorothioate oligonucleotides, and phosphoramidate oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C). (Wagner, Nat. Medicine 1: 1116, 1995; Varga et al., Immun. Lett. 69: 217, 1999; Neilsen, Curr. Opin. Biotech. 10: 71, 1999; Woolf, Nucleic Acids Res. 18: 1763, 1990).

The invention provides a method of inhibiting expression of a gene encoding chronological life span disease or disorder or disease related to aging comprising the step of (i) providing a biological system in which expression of a gene encoding a chronological life span protein is to be inhibited; and (ii) contacting the system with an antisense molecule that hybridizes to a transcript encoding the chronological life span molecule or chronological life span protein. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression can be inducible and/or tissue or cell type-specific. The antisense molecule can be an oligonucleotide or a longer nucleic acid molecule. The invention provides such antisense molecules.

### 24. Inhibitory Ribozymes

The invention provides ribozymes capable of binding message which can inhibit polypeptide activity by targeting mRNA, e.g., inhibition of polypeptides with chronological life span activity. Thus, RNA and DNA enzymes can be designed to cleave to any RNA molecule, thereby increasing its rate of degradation. (Cotten and Birnstiel, EMBO J. 8: 3861-3866, 1989; Usman et al., Nucl. Acids Mol. Biol. 10: 243, 1996; Usman et al., Curr. Opin. Struct. Biol. 1: 527, 1996; Sun et al., Pharmacol. Rev., 52: 325, 2000).

Strategies for designing ribozymes and selecting the protein-specific antisense sequence for targeting are well described in the scientific and patent literature, and the skilled artisan can design such ribozymes using the novel reagents of the invention.

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but can also be formed in the motif of a hairpin, hepatitis delta virus, group I intron or RnaseP-like RNA (in association with an RNA guide sequence). Examples of such hammerhead motifs are described by Rossi, Aids Research and Human Retroviruses 8: 183, 1992; hairpin motifs by Hampel, Biochemistry 28: 4929, 1989, and Hampel, Nuc. Acids Res. 18: 299, 1990; the hepatitis delta virus motif by Perrotta, Biochemistry 31: 16, 1992; the RnaseP motif by Guerrier-Takada, Cell 35: 849, 1983; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

The invention provides a method of inhibiting expression of a gene encoding a chronological life span gene (such as comprising the step of (i) providing a biological system in which expression of a gene encoding a chronological life span protein is to be inhibited; and (ii) contacting the system with a ribozyme that hybridizes to a transcript encoding the chronological life span molecule or chronological life span protein and directs cleavage of the transcript. According to certain embodiments of the invention the biological system comprises a cell, and the contacting step comprises expressing the ribozyme in the cell. According to certain embodiments of the invention the biological system comprises a subject, e.g., a mammalian subject such as a mouse or human, and the contacting step comprises administering the ribozyme to the subject or comprises expressing the ribozyme in the subject. The expression can be inducible and/or tissue or cell-type specific according to certain embodiments of the invention. The invention provides ribozymes designed to cleave transcripts encoding chronological life span molecules or chronological life span proteins, or polymorphic variants thereof, as described above.

### 25. Chronological Life Span Transgenic and “Knockout” Non-Human Animals

The invention provides transgenic non-human animals comprising a nucleic acid, a polypeptide, an expression cassette or vector or a transfected or transformed cell of the invention. The transgenic non-human animals can be, e.g., goats, rabbits, sheep, pigs, cows, rats and mice, comprising the nucleic acids of the invention. A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. The transgenic DNA can encode mammalian kinases. Native expression in an animal can be reduced by providing an amount of antisense RNA or DNA effective to reduce expression of the receptor.

These animals can be used, e.g., as in vivo models to study which is modulators of a chronological life span-signaling activity, or, as models to screen for agents that change the chronological life span-signaling activity in vivo.

In one aspect, the inserted transgenic sequence is a sequence of the invention designed such that it does not express a functional chronological life span polypeptide. The defect can be designed to be on the transcriptional, translational and/or the protein level.

The coding sequences for the polypeptides, the chronological life span polypeptides, or mutant polypeptide to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock, J. Immunol. Methods 231: 147-157, 1999, describing the production of recombinant proteins in the milk of transgenic dairy animals; Baguisi, Nat. Biotechnol. 17: 456-461, 1999, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP). One exemplary method to produce genetically altered non-human animals is to genetically modify embryonic stem cells. The modified cells are injected into the blastocoel of a blastocyst. This is then grown in the uterus of a pseudopregnant female. In order to readily detect chimeric progeny, the blastocysts can be obtained from a different parental line than the embryonic stem cells. For example, the blastocysts and embryonic stem cells can be derived from parental lines with different hair color or other readily observable phenotype. The resulting chimeric animals can be bred in order to obtain non-chimeric animals which have received the modified genes through germ-line transmission. Techniques for the introduction of embryonic stem cells into blastocysts and the resulting generation of transgenic animals are well known.

Because cells contain more than one copy of a gene, the cell lines obtained from a first round of targeting are likely to be heterozygous for the targeted allele. Homozygosity, in which both alleles are modified, can be achieved in a number of ways. In one approach, a number of cells in which one copy has been modified are grown. They are then subjected to another round of targeting using a different selectable marker. Alternatively, homozygotes can be obtained by breeding animals heterozygous for the modified allele, according to traditional Mendelian genetics. In some situations, it can be desirable to have two different modified alleles. This can be achieved by successive rounds of gene targeting or by breeding heterozygotes, each of which carries one of the desired modified alleles. See, e.g., U.S. Pat. No. 5,789,215.

A variety of methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division. (Brinster et al., Proc. Nat. Acad. Sci. USA 82: 4438-4442, 1985). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), Harlan Sprague Dawley (Indianapolis, Ind.), and the like

The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout, Experientia 47: 897-905, 1991. Other procedures for introduction of DNA into tissues of animals are described in U.S. Pat. No. 4,945,050 (Sanford et al., Jul. 30, 1990).

By way of example only, to prepare a transgenic mouse, female mice are induced to superovulate. Females are placed with males, and the mated females are sacrificed by CO.sub.2 asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice. (Hammer et al., Cell 63: 1099-1112, 1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (Teratocarcinomas and Embryonic Stem Cells, A Practical Approach, E. J. Robertson, ed., IRL Press, 1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombination. (Capecchi, Science 244: 1288-1292, 1989). Methods for positive selection of the recombination event (i.e., neo resistance) and dual positive-negative selection (i.e., neo resistance and gancyclovir resistance) and the subsequent identification of the desired clones by PCR have been described by Capecchi, supra and Joyner et al., Nature 338: 153-156, 1989, the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others. (Houdebine and Chourrout, supra; Pursel et al., Science 244: 1281-1288, 1989; and Simms et al., Bio/Technology 6: 179-183, 1988).

### 26. Chronological Life Span Functional Knockouts

The invention provides non-human animals that do not express their endogenous chronological life span polypeptides, or, express their endogenous chronological life span polypeptides at lower than wild type levels (thus, while not completely “knocked out” their chronological life span activity is functionally “knocked out”). The invention also provides “knockout animals” and methods for making and using them. For example, in one aspect, the transgenic or modified animals of the invention comprise a “knockout animal,” e.g., a “knockout mouse,” engineered not to express an endogenous gene, e.g., an endogenous chronological life span gene, which is replaced with a gene expressing a polypeptide of the invention, or, a fusion protein comprising a polypeptide of the invention. Thus, in one aspect, the inserted transgenic sequence is a sequence of the invention designed such that it does not express a functional chronological life span polypeptide. The defect can be designed to be on the transcriptional, translational and/or the protein level. Because the endogenous chronological life span gene has been “knocked out,” only the inserted polypeptide of the invention is expressed.

A “knock-out animal” is a specific type of transgenic animal having cells that contain DNA containing an alteration in the nucleic acid sequence that reduces the biological activity of the polypeptide normally encoded therefrom by at least 80% compared to the unaltered gene. The alteration can be an insertion, deletion, frameshift mutation, missense mutation, introduction of stop codons, mutation of critical amino acid residue, removal of an intron junction, and the like. Preferably, the alteration is an insertion or deletion, or is a frameshift mutation that creates a stop codon. Typically, the disruption of specific endogenous genes can be accomplished by deleting some portion of the gene or replacing it with other sequences to generate a null allele. Cross-breeding mammals having the null allele generates a homozygous mammals lacking an active copy of the gene.

A number of such mammals have been developed, and are extremely helpful in medical development. For example, U.S. Pat. No. 5,616,491 describes knock-out mice having suppression of CD28 and CD45. Procedures for preparation and manipulation of cells and embryos are similar to those described above with respect to transgenic animals, and are well known to those of ordinary skill in the art.

A knock out construct refers to a uniquely configured fragment of nucleic acid which is introduced into a stem cell line and allowed to recombine with the genome at the chromosomal locus of the gene of interest to be mutated. Thus, a given knock out construct is specific for a given gene to be targeted for disruption. Nonetheless, many common elements exist among these constructs and these elements are well known in the art. A typical knock out construct contains nucleic acid fragments of about 0.5 kb to about 10.0 kb from both the 5′ and the 3′ ends of the genomic locus which encodes the gene to be mutated. These two fragments are typically separated by an intervening fragment of nucleic acid which encodes a positive selectable marker, such as the neomycin resistance gene. The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for the gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug.

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgenic animal which expresses a polypeptide of the targeted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of the polypeptide product of the wild-type gene from which it was derived.

Each knockout construct to be inserted into the cell must first be in the linear form. Therefore, if the knockout construct has been inserted into a vector, linearization is accomplished by digesting the DNA with a suitable restriction endonuclease selected to cut only within the vector sequence and not within the knockout construct sequence. For insertion, the knockout construct is added to the ES cells under appropriate conditions for the insertion method chosen, as is known to the skilled artisan. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion can be accomplished in a variety of ways known to the skilled artisan, however a preferred method is by microinjection. For microinjection, about 10-30 cells are collected into a micropipette and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan. After the ES cell has been introduced into the embryo, the embryo can be implanted into the uterus of a pseudopregnant foster mother for gestation as described above.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a target gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

The functional chronological life span “knockout” non-human animals of the invention are of several types. Some non-human animals of the invention that are functional chronological life span “knockouts” express sufficient levels of a chronological life span inhibitory nucleic acid, e.g., antisense sequences or ribozymes of the invention, to decrease the levels or knockout the expression of functional polypeptide. Some non-human animals of the invention that are functional chronological life span “knockouts” express sufficient levels of a chronological life span dominant negative polypeptide such that the effective amount of free endogenous active chronological life span is decreased. Some non-human animals of the invention that are functional chronological life span “knockouts” express sufficient levels of an antibody of the invention, e.g., a chronological life span antibody, such that the effective amount of free endogenous active chronological life span protein is decreased. Some non-human animals of the invention that are functional chronological life span “knockouts” are “conventional” knockouts in that their endogenous chronological life span gene has been disrupted or mutated.

Functional chronological life span “knockout” non-human animals of the invention also include the inbred mouse strain of the invention and the cells and cell lines derived from these mice.

The invention provides methods for treating a subject with a chronological life span related disease or disorder. The method comprises providing an inhibitor of a chronological life span activity, e.g., a nucleic acid (e.g., antisense, ribozyme) or a polypeptide (e.g., antibody or dominant negative) of the invention. The inhibitor is administered in sufficient amounts to the subject to inhibit the expression of chronological life span polypeptides.

### 27. Chronological Life Span Inbred Mouse Strains

The invention provides an inbred mouse and an inbred mouse strain that can be generated as described herein and bred by standard techniques, see, e.g., U.S. Pat. Nos. 6,040,495; 5,552,287.

In order to screen for mutations with recessive effects a number of strategies can be used, all involving a further two generations. For example, male G1 mice can be bred to wild-type female mice. The resulting progeny (G2 mice) can be interbred or bred back to the G1 father. The G3 mice that result from these crosses will be homozygotes for mutations in a small number of genes (3-6) in the genome, but the identity of these genes is unknown. With enough G3 mice, a good sampling of the genome should be present.

### 28. Peptides and Polypeptides

The invention provides isolated or recombinant polypeptides comprising an amino acid sequence having at least 95%, 96%, 97%, 98%, 99% or more sequence identity to a sequence of any gene indicated in Table 1 or in Table 2, over a region of at least about 100, 150, 200, 250, 300, 350, 400, 450, 500, 550, 600, 650, 700, 750, 800, 850, 900, 950, 1000, 1100 or more residues, or, the full length of the polypeptide, or, a polypeptide encoded by a nucleic acid of the invention. In one aspect, the polypeptide comprises sequences for the genes indicated in Table 1 or in Table 2. The invention provides methods for inhibiting the activity of chronological life span polypeptides, e.g., a polypeptide of the invention. The invention also provides methods for screening for compositions that inhibit the activity of, or bind to (e.g., bind to the active site), of chronological life span polypeptides, e.g., a polypeptide of the invention.

In one aspect, the invention provides chronological life span polypeptides (and the nucleic acids encoding them) where one, some or all of the chronological life span polypeptides replacement with substituted amino acids. In one aspect, the invention provides methods to disrupt the interaction of chronological life span polypeptides with other proteins, in antigen presentation pathways.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for modulators of a chronological life span activity and for agents that can ameliorate a a chronological life span disease or disorder or related disorder or a disease or disorder associated with aging. Polypeptides (e.g., antibody or dominant negative) of the invention can also be used to tolerize a subject to an antigen for, e.g., inducing humoral or cellular anergy to an immunogen.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, it has an a chronological life span-signaling activity. A mimetic composition can also be within the scope of the invention if it can inhibit an activity of a chronological life span polypeptides of the invention, e.g., be a dominant negative mutant or, bind to an antibody of the invention.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(.dbd.O)—CH.sub.2-for —C(.dbd.O)—NH—), aminomethylene (CH.sub.2-NH), ethylene, olefin (CH.dbd.CH), ether (CH.sub.2-O), thioether (CH.sub.2-S), tetrazole (CN.sub.4-), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (□adioimmu)-acetic acid, or (□adioimmu)alkyl-acetic acid, where alkyl is defined above. Nitrile derivative (e.g., containing the CN-moiety in place of COOH) can be substituted for □adioimmuno or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of □adioim include, e.g., pipecolic acid, thiazolidine carboxylic acid, 3- or 4-hydroxy □adioim, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of □adioim and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for radioimmunoassay). One or more amino acids can be deleted, for example, from a chronological life span polypeptide of the invention, resulting in modification of the structure of the polypeptide, without significantly altering its biological activity. For example, amino- or carboxyl-terminal, or internal, amino acids which are not required for a chronological life span-signaling activity can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site. (See e.g., Williams, Biochemistry 34: 1787-1797, 1995; Dobeli, Protein Expr. Purif. 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll, DNA Cell. Biol., 12: 441-53, 1993.

The terms “polypeptide” and “protein” as used herein, refer to amino acids joined to each other by peptide bonds or modified peptide bonds, i.e., peptide isosteres, and can contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

As used herein, the term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

Exemplary chronological life span genes, their mammalian orthologs, and identified sequences are shown in Table 1 and in Table 2. One of skill in the art can determine their nucleic acid and amino acid translation by referencing their corresponding GenBank accession number in databases.

### 29. Fusion Proteins

Antibodies to chronological life span gene products (e.g., a chronological life span protein) can be used to generate fusion proteins. For example, the antibodies of the present invention, when fused to a second protein, can be used as an antigenic tag. Antibodies raised against a chronological life span gene product (e.g., a chronological life span protein) can be used to indirectly detect the second protein by binding to the polypeptide.

Examples of domains that can be fused to polypeptides include not only heterologous signal sequences, but also other heterologous functional regions. The fusion does not necessarily need to be direct, but can occur through linker sequences.

Moreover, fusion proteins can also be engineered to improve characteristics of the polypeptide. For instance, a region of additional amino acids, particularly charged amino acids, can be added to the N-terminus of the polypeptide to improve stability and persistence during purification from the host cell or subsequent handling and storage. Also, peptide moieties can be added to the polypeptide to facilitate purification. Such regions can be removed prior to final preparation of the polypeptide. The addition of peptide moieties to facilitate handling of polypeptides are familiar and routine techniques in the art.

Moreover, antibody compositions to a chronological life span proteins, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. EP A 394,827; Traunecker et al., Nature, 331: 84-86, 1988. Fusion proteins having disulfide-linked dimeric structures (due to the IgG) can also be more efficient in binding and neutralizing other molecules, than the monomeric secreted protein or protein fragment alone. Fountoulakis et al., J. Biochem. 270: 3958-3964, 1995.

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion can hinder therapy and diagnosis if the fusion protein is used as an antigen for immunizations. In drug discovery, for example, human proteins, such as hIL-5, have been fused with Fc portions for the purpose of high throughput screening assays to identify antagonists of hIL-5. Bennett et al., J. Molecular Recognition 8: 52-58, 1995; Johanson et al., J. Biol. Chem., 270: 9459-9471, 1995.

Moreover, the polypeptides can be fused to marker sequences, such as a peptide which facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824, 1989, for instance, hexa-histidine provides for convenient purification of the fusion protein. Another peptide tag useful for purification, the “HA” tag, corresponds to an epitope derived from the influenza hemagglutinin protein. Wilson et al., Cell 37: 767, 1984.

Thus, any of these above fusions can be engineered using the polynucleotides or the polypeptides of the present invention.

### 30. Screening Methodologies

In practicing the methods of the invention, a variety of apparatus and methodologies can be used to in conjunction with the polypeptides and nucleic acids of the invention, e.g., to screen polypeptides for chronological life span-signaling activity, to screen compounds as potential modulators (e.g., inhibitors or activators) of a chronological life span activity, e.g., an chronological life span-signaling activity, for antibodies that bind to a polypeptide of the invention, for nucleic acids that hybridize to a nucleic acid of the invention, to screen for cells expressing a polypeptide of the invention and the like.

In one aspect, the peptides and polypeptides of the invention can be bound to a solid support. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of peptides to a solid support can be direct (i.e., the protein contacts the solid support) or indirect (a particular compound or compounds are bound to the support and the target protein binds to this compound rather than the solid support). Peptides can be immobilized either covalently (e.g., utilizing single reactive thiol groups of cysteine residues (see, e.g., Collioud et al., Bioconjugate Chem. 4: 528-536, 1993) or non-covalently but specifically (e.g., via immobilized antibodies (see, e.g., Schuhmann, Adv. Mater. 3: 388-391, 1991; Lu, Anal. Chem. 67: 83-87, 1995; the biotin/strepavidin system (see, e.g., Iwane, Biophys. Biochem. Res. Comm. 230: 76-80, 1997); metal chelating, e.g., Langmuir-Blodgett films (see, e.g., Ng, Langmuir 11: 4048-55, 1995); metal-chelating self-assembled monolayers (see, e.g., Sigal, Anal. Chem. 68: 490-497, 1996) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl (4-iodoacetyl)aminobenzoate (SLAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyldithio)propiona- to (SPDP) (Pierce Chemicals, Rockford, Ill.).

Antibodies can be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon, Nature 377: 525-531, 1989.)

### 31. Arrays or “Biochips”

The invention provides methods for identifying/screening for modulators (e.g., inhibitors, activators) of a chronological life span activity, e.g., chronological life span-signaling activity, using arrays. Potential modulators, including small molecules, nucleic acids, polypeptides (including antibodies) can be immobilized to arrays. Nucleic acids or polypeptides of the invention can be immobilized to or applied to an array. Arrays can be used to screen for or monitor libraries of compositions (e.g., small molecules, antibodies, nucleic acids, and the like) for their ability to bind to or modulate the activity of a nucleic acid or a polypeptide of the invention, e.g., a chronological life span activity. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays can be used to simultaneously quantify a plurality of proteins. Small molecule arrays can be used to simultaneously analyze a plurality of chronological life span modulating or binding activities.

The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts. In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston, Curr. Biol. 8: R171-R174, 1998; Schummer, Biotechniques 23: 1087-1092, 1997; Kern, Biotechniques 23: 120-124, 1997; Solinas-Toldo, Genes, Chromosomes & Cancer 20: 399-407, 1997; Bowtell, Nature Genetics Supp. 21: 25-32, 1999. See also published U.S. patent applications Nos. 20010018642; 20010019827; 20010016322; 20010014449; 20010014448; 20010012537; 20010008765.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface.

A. Supports

Supports can be made of a variety of materials, such as glass, silica, plastic, nylon or nitrocellulose. Supports are preferably rigid and have a planar surface. Supports typically have from 1-10,000,000 discrete spatially addressable regions, or cells. Supports having 10-1,000,000 or 100-100,000 or 1000-100,000 cells are common. The density of cells is typically at least 1000, 10,000, 100,000 or 1,000,000 cells within a square centimeter. Typically a single probe per cell. In some supports, all cells are occupied by pooled mixtures of probes. In other supports, some cells are occupied by pooled mixtures of probes, and other cells are occupied, at least to the degree of purity obtainable by synthesis methods, by a single type of polynucleotide. The strategies for probe design described in the present application can be combined with other strategies, such as those described by WO 95/11995, EP 717,113 and WO 97/29212 in the same array.

The location and sequence of each different polynucleotide probe in the array is generally known. Moreover, the large number of different probes can occupy a relatively small area providing a high density array having a probe density of generally greater than about 60, more generally greater than about 100, and most generally greater than about 600, often greater than about 1000, more often greater than about 5,000, most often greater than about 10,000, preferably greater than about 40,000 more preferably greater than about 100,000, and most preferably greater than about 400,000 different polynucleotide probes per cm². The small surface area of the array (often less than about 10 cm², preferably less than about 5 cm² more preferably less than about 2 cm², and most preferably less than about 1.6 cm²) permits the use of small sample volumes and extremely uniform hybridization conditions.

B. Synthesis of Probe Arrays

Arrays of probes can be synthesized in a step-by-step manner on a support or can be attached in presynthesized form. A preferred method of synthesis is VLSIPS™ (see Fodor et al., Nature 364: 555-556, 1993; McGall et al., U.S. Ser. No. 08/445,332; U.S. Pat. No. 5,143,854; EP 476,014), which entails the use of light to direct the synthesis of polynucleotide probes in high-density, miniaturized arrays. Algorithms for design of masks to reduce the number of synthesis cycles are described by Hubbel et al., U.S. Pat. No. 5,571,639 and U.S. Pat. No. 5,593,839. Arrays can also be synthesized in a combinatorial fashion by delivering monomers to cells of a support by mechanically constrained flowpaths. See Winkler et al., EP 624,059. Arrays can also be synthesized by spotting monomers reagents on to a support using an ink jet printer. See id.; Pease et al., EP 728,520.

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Appropriate scanning devices are described by e.g., Trulson et al., U.S. Pat. No. 5,578,832; Stem et al., U.S. Pat. No. 5,631,734 and are available from Affymetrix, Inc., under the GeneChip™ label. Some types of label provide a signal that can be amplified by enzymatic methods. (see Broude et al., Proc. Natl. Acad. Sci. U.S.A. 91: 3072-3076, 1994)

C. Design of Arrays

(1) Customized and Generic Arrays.

The design of arrays for expression monitoring is generally described, for example, WO 97/27317 and WO 97/10365 (these references are herein incorporated by reference). There are two principal categories of arrays. One type of array detects the presence and/or levels of particular mRNA sequences that are known in advance. In these arrays, polynucleotide probes can be selected to hybridize to particular preselected subsequences of mRNA gene sequence. Such expression monitoring arrays can include a plurality of probes for each mRNA to be detected. For analysis of mRNA nucleic acids, the probes are designed to be complementary to the region of the mRNA that is incorporated into the nucleic acids (i.e., the 3′ end). The array can also include one or more control probes.

Generic arrays can include all possible nucleotides of a given length; that is, polynucleotides having sequences corresponding to every permutation of a sequence. Thus since the polynucleotide probes of this invention preferably include up to 4 bases (A, G, C, T) or (A, G, C, U) or derivatives of these bases, an array having all possible nucleotides of length X contains substantially 4.sup.X different nucleic acids (e.g., 16 different nucleic acids for a 2 mer, 64 different nucleic acids for a 3 mer, 65536 different nucleic acids for an 8 mer). Some small number of sequences can be absent from a pool of all possible nucleotides of a particular length due to synthesis problems, and inadvertent cleavage). An array comprising all possible nucleotides of length X refers to an array having substantially all possible nucleotides of length X. All possible nucleotides of length X includes more than 90%, typically more than 95%, preferably more than 98%, more preferably more than 99%, and most preferably more than 99.9% of the possible number of different nucleotides. Generic arrays are particularly useful for comparative hybridization analysis between two mRNA populations or nucleic acids derived therefrom.

(2) Variations

Either customized or generic probe arrays can contain control probes in addition to the probes described above.

(a) Normalization Controls. Normalization controls are typically perfectly complementary to one or more labeled reference polynucleotides that are added to the nucleic acid sample. The signals obtained from the normalization controls after hybridization provide a control for variations in hybridization conditions, label intensity, reading and analyzing efficiency and other factors that can cause the signal of a perfect hybridization to vary between arrays. Signals (e.g., fluorescence intensity) read from all other probes in the array can be divided by the signal (erg., fluorescence intensity) from the control probes thereby normalizing the measurements.

Virtually any probe can serve as a normalization control. However, hybridization efficiency can vary with base composition and probe length. Normalization probes can be selected to reflect the average length of the other probes present in the array, however, they can also be selected to cover a range of lengths. The normalization control(s) can also be selected to reflect the (average) base composition of the other probes in the array. However one or a fewer normalization probes can be used and they can be selected such that they hybridize well (i.e., no secondary structure) and do not match any target-specific probes.

Normalization probes can be localized at any position in the array or at multiple positions throughout the array to control for spatial variation in hybridization efficiently. The normalization controls can be located at the corners or edges of the array as well as in the middle of the array.

(b) Expression Level Controls. Expression level controls can be probes that hybridize specifically with constitutively expressed genes in the biological sample. Expression level controls can be designed to control for the overall health and metabolic activity of a cell. Examination of the covariance of an expression level control with the expression level of the target nucleic acid can indicate whether measured changes or variations in expression level of a gene is due to changes in transcription rate of that gene or to general variations in health of the cell. Thus, for example, when a cell is in poor health or lacking a critical metabolite the expression levels of both an active target gene and a constitutively expressed gene are expected to decrease. The converse can also be true. Thus where the expression levels of both an expression level control and the target gene appear to both decrease or to both increase, the change can be attributed to changes in the metabolic activity of the cell as a whole, not to differential expression of the target gene in question. Conversely, where the expression levels of the target gene and the expression level control do not covary, the variation in the expression level of the target gene can be attributed to differences in regulation of that gene and not to overall variations in the metabolic activity of the cell.

Virtually any constitutively expressed gene can provide a suitable target for expression level controls. Typically expression level control probes can have sequences complementary to subsequences of constitutively expressed genes including, but not limited to the B-actin gene, the transferrin receptor gene, the GAPDH gene, and the like.

(c) Mismatch Controls. Mismatch controls can also be provided for the probes to the target genes, for expression level controls or for normalization controls. Mismatch controls are typically employed in customized arrays containing probes matched to known mRNA species. For example, some such arrays contain a mismatch probe corresponding to each match probe. The mismatch probe is the same as its corresponding match probe except for at least one position of mismatch. A mismatched base is a base selected so that it is not complementary to the corresponding base in the target sequence to which the probe can otherwise specifically hybridize. One or more mismatches are selected such that under appropriate hybridization conditions (e.g. stringent conditions) the test or control probe can be expected to hybridize with its target sequence, but the mismatch probe cannot hybridize (or can hybridize to a significantly lesser extent). Mismatch probes can contain a central mismatch. Thus, for example, where a probe is a 20 mer, a corresponding mismatch probe can have the identical sequence except for a single base mismatch (e.g., substituting a G, a C or a T for an A) at any of positions 6 through 14 (the central mismatch).

In generic (e.g., random, arbitrary, or haphazard) arrays, since the target nucleic acid(s) are unknown perfect match and mismatch probes cannot be a priori determined, designed, or selected. In this instance, the probes can be provided as pairs where each pair of probes differ in one or more preselected nucleotides. Thus, while it is not known a priori which of the probes in the pair is the perfect match, it is known that when one probe specifically hybridizes to a particular target sequence, the other probe of the pair can act as a mismatch control for that target sequence. The perfect match and mismatch probes need not be provided as pairs, but can be provided as larger collections (e.g., 3, 4, 5, or more) of probes that differ from each other in particular preselected nucleotides.

In both customized and generic arrays mismatch probes can provide a control for non-specific binding or cross-hybridization to a nucleic acid in the sample other than the target to which the probe is complementary. Mismatch probes thus can indicate whether a hybridization is specific or not. For example, if the complementary target is present the perfect match probes can be consistently brighter than the mismatch probes. In addition, if all central mismatches are present, the mismatch probes can be used to detect a mutation. Finally, the difference in intensity between the perfect match and the mismatch probe (I(PM)-I(MM)) can provide a good measure of the concentration of the hybridized material.

(d) Sample Preparation, Amplification, and Quantitation Controls. Arrays can also include sample preparation/amplification control probes. These can be probes that are complementary to subsequences of control genes selected because they do not normally occur in the nucleic acids of the particular biological sample being assayed. Suitable sample preparation/amplification control probes can include, for example, probes to bacterial genes (e.g., Bio B) where the sample in question is a biological sample from a eukaryote.

The RNA sample can then be spiked with a known amount of the nucleic acid to which the sample preparation/amplification control probe is directed before processing. Quantification of the hybridization of the sample preparation/amplification control probe can then provide a measure of alteration in the abundance of the nucleic acids caused by processing steps (e.g., PCR, reverse transcription, or in vitro transcription).

Quantitation controls can be similar. Typically they can be combined with the sample nucleic acid(s) in known amounts prior to hybridization. They are useful to provide a quantitation reference and permit determination of a standard curve for quantifying hybridization amounts (concentrations).

E. Methods of Detection

In one method of detection, mRNA or nucleic acid derived therefrom, typically in denatured form, are applied to an array. The component strands of the nucleic acids hybridize to complementary probes, which are identified by detecting label. Optionally, the hybridization signal of matched probes can be compared with that of corresponding mismatched or other control probes. Binding of mismatched probe serves as a measure of background and can be subtracted from binding of matched probes. A significant difference in binding between a perfectly matched probes and a mismatched probes signifies that the nucleic acid to which the matched probes are complementary is present. Binding to the perfectly matched probes is typically at least 1.2, 1.5, 2, 5 or 10 or 20 times higher than binding to the mismatched probes.

In a variation of the above method, nucleic acids are not labeled but are detected by template-directed extension of a probe hybridized to a nucleic acid strand with the nucleic acid strand serving as a template. The probe is extended with a labeled nucleotide, and the position of the label indicates, which probes in the array have been extended. By performing multiple rounds of extension using different bases bearing different labels, it is possible to determine the identity of additional bases in the tag than are determined through complementarity with the probe to which the tag is hybridized. The use of target-dependent extension of probes is described by U.S. Pat. No. 5,547,839.

In a further variation, probes can be extended with inosine. The inosine strand can be labeled. The addition of degenerate bases, such as inosine (it can pair with all other bases), can increase duplex stability between the polynucleotide probe and the denatured single stranded DNA nucleic acids. The addition of 1-6 inosines onto the end of the probes can increase the signal intensity in both hybridization and ligation reactions on a generic ligation array. This can allow for ligations at higher temperatures. The use of degenerate bases is described in WO 97/27317.

Ligation reactions can offer improved discriminate between fully complementary hybrids and those that differ by one or more base pairs, particularly in cases where the mismatch is near the 5′ terminus of the polynucleotide probes. Use of a ligation reaction in signal detection increases the stability of the hybrid duplex, improves hybridization specificity (particularly for shorter polynucleotide probes (e.g., 5 to 12-mers), and optionally, provides additional sequence information. Ligation reactions used in signal detection are described in WO 97/27317. Optionally, ligation reactions can be used in conjunction with template-directed extension of probes, either by inosine or other bases.

F. Analysis of Hybridization Patterns

The position of label is detected for each probe in the array using a reader, such as described by U.S. Pat. No. 5,143,854, WO 90/15070, and Trulson et al., supra. For customized arrays, the hybridization pattern can then be analyzed to determine the presence and/or relative amounts or absolute amounts of known mRNA species in samples being analyzed as described in e.g., WO 97/10365. Comparison of the expression patterns of two samples is useful for identifying mRNAs and their corresponding genes that are differentially expressed between the two samples.

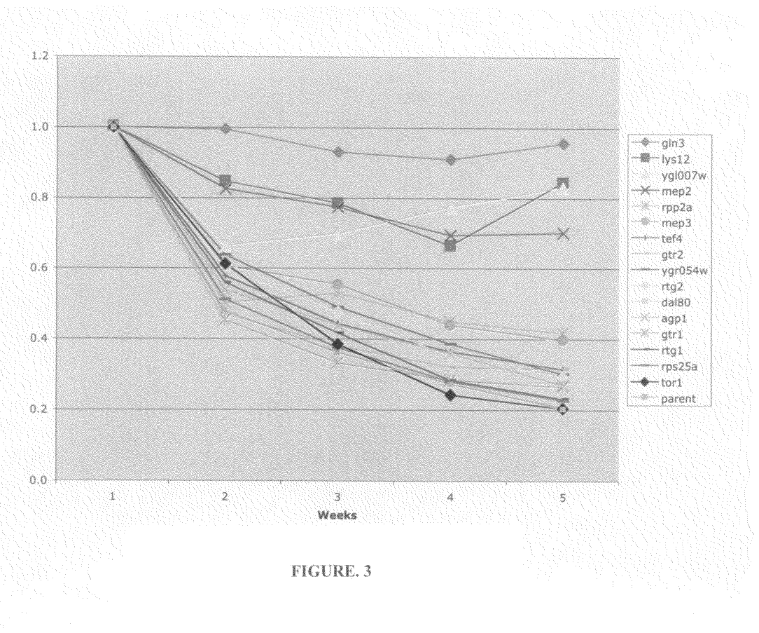

The quantitative monitoring of expression levels for large numbers of genes can prove valuable in elucidating gene function, exploring the causes and mechanisms of disease, and for the discovery of potential therapeutic and diagnostic targets. Expression monitoring can be used to monitor the expression (transcription) levels of nucleic acids whose expression is altered in a disease state. For example, a chronological life span gene can be characterized by the overexpression of a particular marker such as the genes listed in FIG. 3 or in Table 2.

Expression monitoring can be used to monitor expression of various genes in response to defined stimuli, such as a drug. This is especially useful in drug research if the end point description is a complex one, not simply asking if one particular gene is overexpressed or underexpressed. Therefore, where a disease state or the mode of action of a drug is not well characterized, the expression monitoring can allow rapid determination of the particularly relevant genes.

In generic arrays, the hybridization pattern is also a measure of the presence and abundance of relative mRNAs in a sample, although it is not immediately known, which probes correspond to which mRNAs in the sample.

However the lack of knowledge regarding the particular genes does not prevent identification of useful therapeutics. For example, if the hybridization pattern on a particular generic array for a healthy cell is known and significantly different from the pattern for a diseased cell, then libraries of compounds can be screened for those that cause the pattern for a diseased cell to become like that for the healthy cell. This provides a detailed measure of the cellular response to a drug.

Generic arrays can also provide a powerful tool for gene discovery and for elucidating mechanisms underlying complex cellular responses to various stimuli. For example, generic arrays can be used for expression fingerprinting. Suppose it is found that the mRNA from a certain cell type displays a distinct overall hybridization pattern that is different under different conditions (e.g., when harboring mutations in particular genes, in a disease state). Then this pattern of expression (an expression fingerprint), if reproducible and clearly differentiable in the different cases can be used as a very detailed diagnostic. It is not required that the pattern be fully interpretable, but just that it is specific for a particular cell state (and preferably of diagnostic and/or prognostic relevance).

Both customized and generic arrays can be used in drug safety studies. For example, if one is making a new antibiotic, then it should not significantly affect the expression profile for mammalian cells. The hybridization pattern can be used as a detailed measure of the effect of a drug on cells, for example, as a toxicological screen.

The sequence information provided by the hybridization pattern of a generic array can be used to identify genes encoding mRNAs hybridized to an array. Such methods can be performed using DNA nucleic acids of the invention as the target nucleic acids described in WO 97/27317. DNA nucleic acids can be denatured and then hybridized to the complementary regions of the probes, using standard conditions described in WO 97/27317. The hybridization pattern indicates which probes are complementary to nucleic acid strands in the sample. Comparison of the hybridization pattern of two samples indicates which probes hybridize to nucleic acid strands that derive from mRNAs that are differentially expressed between the two samples. These probes are of particular interest, because they contain complementary sequence to mRNA species subject to differential expression. The sequence of such probes is known and can be compared with sequences in databases to determine the identity of the full-length mRNAs subject to differential expression provided that such mRNAs have previously been sequenced. Alternatively, the sequences of probes can be used to design hybridization probes or primers for cloning the differentially expressed mRNAs. The differentially expressed mRNAs are typically cloned from the sample in which the mRNA of interest was expressed at the highest level. In some methods, database comparisons or cloning is facilitated by provision of additional sequence information beyond that inferable from probe sequence by template dependent extension as described above.

### 32. Combinatorial Chemical Libraries

The invention provides methods for identifying/screening for modulators (e.g., inhibitors, activators) of a chronological life span activity, e.g., a chronological life span-signaling activity. In practicing the screening methods of the invention, a test compound is provided. It can be contacted with a polypeptide of the invention in vitro or administered to a cell of the invention or an animal of the invention in vivo. Compounds are also screened using the compositions, cells, non-human animals and methods of the invention for their ability to ameliorate a chronological life span associated disease or chronological life span a chronological life span disease or disorder associated with aging. Combinatorial chemical libraries are one means to assist in the generation of new chemical compound leads for, e.g., compounds that inhibit a chronological life span-signaling activity or, using a transgenic or a knockout non-human animal of the invention, a compound that can be used to treat or ameliorate a chronological life span associated disease or chronological life span a chronological life span disease or disorder associated with aging, including various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease.

A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, the systematic, combinatorial mixing of 100 interchangeable chemical building blocks results in the theoretical synthesis of 100 million tetrameric compounds or 10 billion pentameric compounds. (See, e.g., Gallop et al., J. Med. Chem. 37: 1233-1250, 1994). Preparation and screening of combinatorial chemical libraries are well known to those of skill in the art, see, e.g., U.S. Pat. Nos. 6,004,617; 5,985,356. Such combinatorial chemical libraries include, but are not limited to, peptide libraries. (see, e.g., U.S. Pat. No. 5,010,175; Furka, Int. J. Pept. Prot. Res. 37: 487-493, 1991; Houghton et al., Nature 354: 84-88, 1991). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs, Proc. Nat. Acad. Sci. USA 90: 6909-6913, 1993), vinylogous polypeptides (see, e.g., Hagihara, J. Amer. Chem. Soc. 114: 6568, 1992), non-peptidal peptidomimetics with a Beta-D-Glucose scaffolding (see, e.g., Hirschmann, J. Amer. Chem. Soc. 114: 9217-9218, 1992), analogous organic syntheses of small compound libraries (see, e.g., Chen, J. Amer. Chem. Soc. 116: 2661, 1994), oligocarbamates (see, e.g., Cho, Science 261:1303, 1993), and/or peptidyl phosphonates (see, e.g., Campbell, J. Org. Chem. 59: 658, 1994). See also Gordon, J. Med. Chem. 37: 1385, 1994; for nucleic acid libraries, peptide nucleic acid libraries, see, e.g., U.S. Pat. No. 5,539,083; for antibody libraries, see, e.g., Vaughn, Nature Biotechnology 14: 309-314, 1996; for carbohydrate libraries, see, e.g., Liang et al., Science 274: 1520-1522, 1996, U.S. Pat. No. 5,593,853; for small organic molecule libraries, see, e.g., for isoprenoids U.S. Pat. No. 5,569,588; for thiazolidinones and metathiazanones, U.S. Pat. No. 5,549,974; for pyrrolidines, U.S. Pat. Nos. 5,525,735 and 5,519,134; for morpholino compounds, U.S. Pat. No. 5,506,337; for benzodiazepines U.S. Pat. No. 5,288,514.

Devices for the preparation of combinatorial libraries are commercially available (see, e.g., U.S. Pat. Nos. 6,045,755; 5,792,431; 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., and the like).

The compounds tested as modulators of chronological life span genes or gene products can be any small organic molecule, or a biological entity, such as a protein, e.g., an antibody or peptide, a sugar, a nucleic acid, e.g., an antisense oligonucleotide or RNAi, or a ribozyme, or a lipid. Alternatively, modulators can be genetically altered versions of a chronological life span protein. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial small organic molecule or peptide library containing a large number of potential therapeutic compounds (potential modulator or ligand compounds). Such “combinatorial chemical libraries” or “ligand libraries” (as described above) are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### 33. Antibodies and Antibody-Based Screening Methods

The invention provides isolated or recombinant antibodies that specifically bind to a polypeptide or nucleic acid of the invention, e.g., chronological life span nucleic acids or polypeptides. These antibodies can be used to isolate, identify or quantify a polypeptide of the invention or related polypeptides. These antibodies can be used to isolate other polypeptides within the scope the invention or other related chronological life span-signaling activity polypeptides.

The antibodies can be used in immunoprecipitation, staining (e.g., FACS), immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, N.Y. (1991); Stites (eds.) BASIC AND CLINICAL IMMUNOLOGY (7^{th} ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler, Nature 256: 495, 1975; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom, Trends Biotechnol. 15: 62-70, 1997; Katz, Annu. Rev. Biophys. Biomol. Struct. 26: 27-45, 1997.

Polypeptides or peptides can be used to generate antibodies which bind specifically to the polypeptides of the invention. The resulting antibodies can be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody can be determined using any of a variety of procedures familiar to those skilled in the art. For example, binding can be determined by labeling the antibody with a detectable label such as a fluorescent agent, an enzymatic label, or a radioisotope. Alternatively, binding of the antibody to the sample can be detected using a secondary antibody having such a detectable label thereon. Particular assays include ELISA assays, sandwich assays, radioimmunoassay, and Western Blots.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies which can bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole, 1985, in Monoclonal Antibodies and Cancer Therapy, Alan R. Liss, Inc., pp. 77-96).

Techniques described for the production of single chain antibodies (see, e.g., U.S. Pat. No. 4,946,778) can be adapted to produce single chain antibodies to the polypeptides of the invention. Alternatively, transgenic mice can be used to express humanized antibodies to these polypeptides or fragments thereof.

Antibodies generated against the polypeptides of the invention can be used in screening for similar polypeptides from other organisms and samples. In such techniques, polypeptides from the organism are contacted with the antibody and those polypeptides which specifically bind the antibody are detected. Any of the procedures described above can be used to detect antibody binding.

### 34. Screening for Effectiveness of Candidate Compounds in Animal Models and Humans

Candidate compounds identified using any of the screening methods described herein (or other suitable methods) can be tested in any appropriate animal model for chronologcial life span diseases or disorders or a disease or disorder associated with aging, including both genetic and pharmacological models. For example, such compounds can be tested in the knockout mouse, transgenic mouse and/or in any of the other models described herein.

When testing compounds in animal models, it can be preferred to use an animal model that does not contain a mutation or deletion in the expected target of the compound (although such animal models can usefully be employed as controls for specificity of the compound since if the compound is similarly effective in such animal models it is most likely acting via a mechanism that does not involve interaction with the expected target). Candidate compounds can also be tested in human subjects suffering from a chronological life span diseases or disorder or a disease or disorder associated with aging.

In general, such tests for efficacy involve administering the candidate compound to the subject (whether animal or human) and observing the subject to determine whether administration of the compound results in amelioration in or reduction of any sign or symptom of, e.g., a chronological life span disease or disorder (or results in a decreased incidence of developing a chronological life span disease or disorder).

In humans, any of the parameters used in the diagnosis and/or assessment of patients suffering from or suspected of suffering from a chronological life span disease or disorder, can be assessed. For example, subjects can be selected by detecting a polymorphic variant of a polymorphism in a coding or noncoding portion of a gene selected from the group consisting of an expression profile gene ortholog set forth in Table 1 or in Table 2 or fragment thereof, or detecting a polymorphic variant of a polymorphism in a genomic region linked to such a gene, obtained from a subject. According to certain embodiments of the invention a group of subjects selected using any of the inventive methods is compared with a group of subjects selected using any other diagnostic criterion.

Thus the invention provides a method for identifying a candidate compound for treatment of a chronological life span disease or disorder or disease or disorder associated with aging comprising steps of: (i) providing a subject or subjects at risk of or exhibiting one or more phenotypes suggestive of a chronological life span disease or disorder or disease or disorder associated with aging, wherein the subject or subjects have an alteration in expression of at least one chronological life span protein; (ii) administering the candidate compound to the subject or subjects; (iii) comparing severity or incidence of the phenotype in the subject or subjects to severity or incidence of the phenotype in a subject or subjects to which the compound is not administered. Typically the method will be performed using groups of animals. If the phenotype appears less severe or occurs at reduced frequency in the subject(s) to which the compound is administered, the compound is identified a candidate compound for the treatment of a a chronological life span disease or disorder or disease or disorder associated with aging susceptibility (although of course this can be confirmed using additional methods). In a preferred embodiment, candidate compounds can be used for the treatment of chronological life span diseases or disorders or related diseases or disorders.

According to certain embodiments of the invention the subject that receive the compound and those that do not receive the compound (i.e., controls) are genetically similar or identical animals. (It is noted that historical controls can be used.). According to certain embodiments of the invention the compound is any compound identified according to any of the inventive compound screening methods described herein.

### 35. Therapeutic Applications

The compounds and modulators identified by the methods of the present invention can be used in a variety of methods of treatment. Thus, the present invention provides compositions and methods for treating chronological life span associated disease or a chronological life span disease or disorder associated with aging, including various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease.

Other exemplary chronological life span diseases/conditions or disorders associated with aging include, but are not limited to, osteoporosis, sarcopenia, obesity, stroke, arthritis, susceptibility to infection and impaired immune function, prostate hyperplasia, hypertension, and infertility.

Preferably, treatment using a polypeptide or polynucleotide of the present invention could either be by administering an effective amount of a polypeptide to the patient, or by removing cells from the patient, supplying the cells with a polynucleotide of the present invention, and returning the engineered cells to the patient (ex vivo therapy).

### 36. Formulation and Administration of Pharmaceutical Compositions

The invention provides pharmaceutical compositions comprising nucleic acids, peptides and polypeptides (including Abs) of the invention. As discussed above, the nucleic acids, peptides and polypeptides of the invention can be used to inhibit or activate expression of an endogenous chronological life span polypeptides. Such inhibition in a cell or a non-human animal can generate a screening modality for identifying compounds to treat or ameliorate a chronological life span disease or disorder associated with aging, including various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease.

The nucleic acids, peptides and polypeptides of the invention can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of nucleic acids, peptides or polypeptides of the invention are dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Nucleic acids, peptides or polypeptides of the invention, when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix, Pharm Res. 13: 1760-1764, 1996; Samanen, J. Pharm. Pharmacol. 48: 119-135, 1996; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. (See, e.g., Sayani, Crit. Rev. Ther. Drug Carrier Syst. 13: 85-184, 1996.) For topical, transdermal administration, the agents are formulated into ointments, creams, salves, powders and gels. Transdermal delivery systems can also include, e.g., patches.

The nucleic acids, peptides or polypeptides of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention. (See, e.g., Putney, Nat. Biotechnol. 16: 153-157, 1998).

For inhalation, the nucleic acids, peptides or polypeptides of the invention can be delivered using any system known in the art, including dry powder aerosols, liquids delivery systems, air jet nebulizers, propellant systems, and the like. See, e.g., Patton, Biotechniques 16: 141-143, 1998; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

The nucleic acids, peptides or polypeptides of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (TT) injections, e.g., to focus on a specific organ, e.g., brain and CNS. (See e.g., Gurun, Anesth Analg. 85: 317-323, 1997). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's. (See also, Bai, J. Neuroimmunol. 80: 65-75, 1997; Warren, J. Neurol. Sci. 152: 31-38, 1997; Tonegawa, J. Exp. Med. 186: 507-515, 1997.)

In one aspect, the pharmaceutical formulations comprising nucleic acids, peptides or polypeptides of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble nucleic acids, peptides or polypeptides of the invention have been attached to the surface of the monolayer or bilayer. For example, peptides can be attached to hydrazide-PEG-(distearoylphosphatidyl)ethanolamine-containing liposomes. (See, e.g., Zalipsky Bioconjug. Chem. 6: 705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the nucleic acid, peptides and/or polypeptides of the invention are incorporated within micelles and/or liposomes. (See, e.g., Suntres, J. Pharm. Pharmacol. 46: 23-28, 1994; Woodle, Pharm. Res. 9: 260-265, 1992). Liposomes and liposomal formulations can be prepared according to standard methods and are also well known in the art. (See, e.g., Remington's; Akimaru, Cytokines Mol. Ther. 1: 197-210, 1995; Alving, Immunol. Rev. 145: 5-31, 1995; Szoka, Ann. Rev. Biophys. Bioeng. 9: 467, 1980, U.S. Pat. Nos. 4,235,871, 4,501,728 and 4,837,028.)

The pharmaceutical compositions are generally formulated as sterile, substantially isotonic and in full compliance with all Good Manufacturing Practice (GMP) regulations of the U.S. Food and Drug Administration.

### 37. Treatment Regimens and Pharmacokinetics

The pharmaceutical compositions of the invention can be administered in a variety of unit dosage forms depending upon the method of administration. Dosages for typical nucleic acid, peptide and polypeptide pharmaceutical compositions are well known to those of skill in the art. Such dosages are typically advisorial in nature and are adjusted depending on the particular therapeutic context, patient tolerance, and the like. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer, Science 249: 1527-1533, 1990.

In therapeutic applications, compositions are administered to a patient suffering from a chronological life span disease or a disease or disorder associated with aging to at least partially arrest the condition or a disease and/or its complications. For example, in one aspect, a soluble peptide pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

The invention provides pharmaceutical compositions comprising one or a combination of antibodies, e.g., antibodies to chronological life span gene products (monoclonal, polyclonal or single chain Fv; intact or binding fragments thereof) or nucleic acid compositions, e.g., antisense oligonucleotides, double stranded RNA oligonucleotides (RNAi) or DNA oligonucleotides (vectors) containing nucleotide sequences encoding for the transcription of shRNA molecules, formulated together with a pharmaceutically acceptable carrier. Some compositions include a combination of multiple (e.g., two or more) monoclonal antibodies or antigen-binding portions thereof of the invention. In some compositions, each of the antibodies or antigen-binding portions thereof of the composition is a monoclonal antibody or a human sequence antibody that binds to a distinct, pre-selected epitope of an antigen.

In prophylactic applications, pharmaceutical compositions or medicaments are administered to a patient susceptible to, or otherwise at risk of a disease or condition (e.g., a chronological life span disease or disorder or a disease related to aging) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved. Typically, any response is monitored and repeated dosages are given if the response starts to wane.

### 38. Effective Dosages

Effective doses of the antibody compositions of the present invention, e.g., antibodies to chronological life span gene products (e.g., chronological life span proteins), or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of chronological life span diseases or disorders or diseases or disorders associated with aging described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with an antibody or nucleic acid composition, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. Antibody is usually administered on multiple occasions. Intervals between single dosages can be weekly, monthly or yearly. Intervals can also be irregular as indicated by measuring blood levels of antibody in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Doses for nucleic acids range from about 10 ng to 1 g, 100 ng to 100 mg, 1 μg to 10 mg, or 30-300 μg DNA per patient. Doses for infectious viral vectors vary from 10-100, or more, virions per dose.

### 39. Routes of Administration

Antibody compositions for inducing an immune response, e.g., antibodies to chronological life span gene products (e.g., chronological life span proteins), or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of treatment of chronological life span diseases or disorders or diseases or disorders associated with aging described herein, can be administered by parenteral, topical, intravenous, oral, subcutaneous, intraarterial, intracranial, intraperitoneal, intranasal or intramuscular means for prophylactic as inhalants for antibody preparations and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue, for example intracranial injection or convection enhanced delivery. Intramuscular injection or intravenous infusion are preferred for administration of antibody. In some methods, particular therapeutic antibodies are delivered directly into the cranium. In some methods, antibodies are administered as a sustained release composition or device, such as a Medipad™ device.

Agents of the invention can optionally be administered in combination with other agents that are at least partly effective in treating various chronological life span diseases or disorders or diseases or disorders associated with aging. In the case of targets in the brain, agents of the invention can also be administered in conjunction with other agents that increase passage of the agents of the invention across the blood-brain barrier (BBB).

### 40. Formulation

Antibody compositions for inducing an immune response, e.g., antibodies to antibodies to chronological life span gene products (e.g., chronological life span proteins), or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of treatment of chronological life span diseases or disorders or diseases or disorders associated with aging described herein, are often administered as pharmaceutical compositions comprising an active therapeutic agent, i.e., and a variety of other pharmaceutically acceptable components. See the most recent edition of Remington's Pharmaceutical Science (e.g., 20^{h} ed., Mack Publishing Company, Easton, Pa., 2000). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990; Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### 41. Toxicity

Preferably, a therapeutically effective dose of the antibody compositions or nucleic acid compositions, e.g., antisense oligonucleotides, double stranded RNA oligonucleotides (RNAi), or DNA oligonucleotides (vectors) containing nucleotide sequences encoding for the transcription of shRNA molecules, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

### 42. Diagnostic Methods

A. Diagnosis of Chronological Life Span Disorders or Chronological Life Span Disorder Susceptibility

The invention provides a variety of methods for the diagnosis of a chronological life span disease or disorder susceptibility or disease or disorder susceptibility related to aging. In particular, the invention provides a method for the diagnosis of chronological life span disease or disorder susceptibility comprising: (i) providing a sample obtained from a subject to be tested for chronological life span disease or disorder susceptibility; and (ii) detecting a polymorphic variant of a polymorphism in a coding or noncoding portion of a gene selected from the group consisting of an expression profile gene ortholog set forth in Table 2 or fragment thereof, or detecting a polymorphic variant of a polymorphism in a genomic region linked to such a gene, in the sample. It is to be understood that “susceptibility to a chronological life span disorder” does not necessarily mean that the subject will develop a chronological life span disorder but rather that the subject is, in a statistical sense, more likely to develop chronological life span disorder than an average member of the population. As used herein, “susceptibility to a chronological life span disorder” can exist if the subject has one or more genetic determinants (e.g., polymorphic variants or alleles) that can, either alone or in combination with one or more other genetic determinants, contribute to an increased risk of developing a chronological life span disorder in some or all subjects. Ascertaining whether the subject has any such genetic determinants (i.e., genetic determinants that can increase the risk of developing a chronological life span disorder in the appropriate genetic background) is included in the concept of diagnosing susceptibility to a chronological life span disorder as used herein. Such determination is useful, for example, for purposes of genetic counseling. Thus providing diagnostic information regarding chronological life span disorder susceptibility includes providing information useful in genetic counseling, and the provision of such information is encompassed by the invention.

The sample itself will typically consist of cells (e.g., blood or brain cells), tissue, and the like, removed from the subject. The subject can be an adult, child, fetus, or embryo. According to certain embodiments of the invention the sample is obtained prenatally, either from the fetus or embryo or from the mother (e.g., from fetal or embryonic cells in that enter the maternal circulation). The sample can be further processed before the detecting step. For example, DNA in the cell or tissue sample can be separated from other components of the sample, can be amplified, and the like. All samples obtained from a subject, including those subjected to any sort of further processing, are considered to be obtained from the subject.

In general, if the polymorphism is located in a gene, it can be located in a noncoding or coding region of the gene. If located in a coding region the polymorphism can, but frequently will not, result in an amino acid alteration. Such alteration can or can not have an effect on the function or activity of the encoded polypeptide. If the polymorphism is linked to, but not located within, a gene, it is preferred that the polymorphism is closely linked to the gene. For example, it is preferred that the recombination frequency between the polymorphism and the gene is less than approximately 20%, preferably less than approximately 10%, less than approximately 5%, less than approximately 1%, or still less.

According to certain preferred embodiments of any of the inventive methods described above, the gene can be coincident with a mapped or identified chronological life span disorder susceptibility locus or a related aging disorder susceptibility locus. For example, according to various embodiments of the invention the gene can encode any of the molecules listed in the tables as shown herein. In a particular embodiment of the invention, discussed further below, the preferred genes encode the genes as set forth in Table 2. The inventive methods also encompass genes coincident with chronological life span disorder susceptibility loci that have yet to be mapped or identified. By “coincident with” is meant either that the gene or a portion thereof falls within the identified chromosomal location or is located in close proximity to that location. In general, the resolution of studies identifying genetic susceptibility loci can be on the order of tens of centimorgans. According to certain embodiments of the invention “close proximity” refers to within 20 centimorgans of either side of the susceptibility locus, more preferably within 10 centimorgans of either side of the susceptibility locus, yet more preferably within 5 centimorgans of either side of the susceptibility locus. In general, susceptibility loci are designated by the chromosomal band positions that they span (e.g., 8p21 refers to chromosome 8, arm p, band 21; 8p20-21 refers to chromosome 8, arm p, bands 20-21 inclusive) and can be defined at higher resolution (e.g., 8p21.1). In general, the terms “coincident with” and “close proximity” can be interpreted in light of the knowledge of one of ordinary skill in the art.

B. Methods and Reagents for Identification and Detection of Polymorphisms

In general, polymorphisms of use in the practice of the invention can be initially identified using any of a number of methods well known in the art. For example, numerous polymorphisms are known to exist and are available in public databases, which can be searched as described herein. Alternately, polymorphisms can be identified by sequencing either genomic DNA or cDNA in the region in which it is desired to find a polymorphism. According to one approach, primers are designed to amplify such a region, and DNA from a subject suffering from a chronological life span is obtained and amplified. The DNA is sequenced, and the sequence (referred to as a “subject sequence”) is compared with a reference sequence, which is typically taken to represent the “normal” or “wild type” sequence. Such a sequence can be, for example, the human draft genome sequence, publicly available in various databases, or a sequence deposited in a database such as GenBank. In general, if sequencing reveals a difference between the sequenced region and the reference sequence, a polymorphism has been identified. Note that this analysis does not necessarily presuppose that either the subject sequence or the reference sequence is the “normal”, most common, or wild type sequence. It is the fact that a difference in nucleotide sequence is identified at a particular site that determines that a polymorphism exists at that site. In most instances, particularly in the case of SNPs, only two polymorphic variants will exist at any location. However, in the case of SNPs, up to four variants can exist since there are four naturally occurring nucleotides in DNA. Other polymorphisms such as insertions can have more than four alleles.

Once a polymorphic site is identified, any of a variety of methods can be employed to detect the existence of any particular polymorphic variant in a subject. In general, a subject can have either the reference sequence or an alternate sequence at the site. The phrase “detecting a polymorphism” or “detecting a polymorphic variant” as used herein generally refers to determining which of two or more polymorphic variants exists at a polymorphic site, although “detecting a polymorphism” can also refer to the process of initially determining that a polymorphic site exists in a population. The meaning to be given to these phrases will be clear from the context as interpreted in light of the knowledge of one of ordinary skill in the art. For purposes of description, if a subject has any sequence other than a defined reference sequence (e.g. the sequence present in the human draft genome) at a polymorphic site, the subject can be said to exhibit the polymorphism. In general, for a given polymorphism, any individual will exhibit either one or two possible variants at the polymorphic site (one on each chromosome). (This can, however, not be the case if the individual exhibits one more chromosomal abnormalities such as deletions.)

Detection of a polymorphism or polymorphic variant in a subject (genotyping) can be performed by sequencing, similarly to the manner in which the existence of a polymorphism is initially established as described above. However, once the existence of a polymorphism is established a variety of more efficient methods can be employed. Many such methods are based on the design of oligonucleotide probes or primers that facilitate distinguishing between two or more polymorphic variants.

“Probes” or “primers”, as used herein, typically refers to oligonucleotides that hybridize in a base-specific manner to a complementary nucleic acid molecule as described herein. Such probes and primers include polypeptide nucleic acids, as described in Nielsen et al., Science 254: 1497-1500, 1991. The term “primer” in particular generally refers to a single-stranded oligonucleotide that can act as a point of initiation of template-directed DNA synthesis using methods such as PCR (polymerase chain reaction), LCR (ligase chain reaction), and the like. Typically, a probe or primer will comprise a region of nucleotide sequence that hybridizes to at least about 8, more often at least about 10 to 15, typically about 20-25, and frequently about 40, 50 or 75, consecutive nucleotides of a nucleic acid molecule. In certain embodiments of the invention, a probe or primer comprises 100 or fewer nucleotides, preferably from 6 to 50 nucleotides, preferably from 12 to 30 nucleotides. In certain embodiments of the invention, the probe or primer is at least 70% identical to the contiguous nucleotide sequence or to the complement of the contiguous nucleotide sequence, preferably at least 80% identical, more preferably at least 90% identical, even more preferably at least 95% identical, or having an even higher degree of identity. In certain embodiments of the invention a preferred probe or primer is capable of selectively hybridizing to a target contiguous nucleotide sequence or to the complement of the contiguous nucleotide sequence. According to certain embodiments of the invention a probe or primer further comprises a label, for example by incorporating a radioisotope, fluorescent compound, enzyme, or enzyme co-factor.

Oligonucleotides that exhibit differential or selective binding to polymorphic sites can readily be designed by one of ordinary skill in the art. For example, an oligonucleotide that is perfectly complementary to a sequence that encompasses a polymorphic site (i.e., a sequence that includes the polymorphic site within it or at one or the other end) will generally hybridize preferentially to a nucleic acid comprising that sequence as opposed to a nucleic acid comprising an alternate polymorphic variant.

In order to detect polymorphisms and/or polymorphic variants, it will frequently be desirable to amplify a portion of DNA encompassing the polymorphic site. Such regions can be amplified and isolated by PCR using oligonucleotide primers designed based on genomic and/or cDNA sequences that flank the site. See e.g., PCR Primer: A Laboratory Manual, Dieffenbach, C. W. and Dveksler, G. S. (eds.); PCR Basics: From Background to Bench, Springer Verlag, 2000; M. J. McPherson, et al; Mattila et al., Nucleic Acids Res. 19: 4967, 1991; Eckert et al., PCR Methods and Applications 1: 17, 1991; PCR (eds. McPherson et al., IRL Press, Oxford); and U.S. Pat. No. 4,683,202. Other amplification methods that can be employed include the ligase chain reaction (LCR) (Wu and Wallace, Genomics 4: 560, 1989, Landegren et al., Science 241: 1077, 1988, transcription amplification (Kwoh et al., Proc. Natl. Acad. Sci. USA, 86: 1173, 1989), self-sustained sequence replication (Guatelli et al., Proc. Nat. Acad. Sci. USA 87: 1874, 1990), and nucleic acid based sequence amplification (NASBA). Guidelines for selecting primers for PCR amplification are well known in the art. See, e.g., McPherson, M., et al., PCR 2000, cited supra. A variety of computer programs for designing primers are available, e.g., “Oligo” (National Biosciences, Inc, Plymouth Minn.), MacVector (Kodak/IBI), and the GCG suite of sequence analysis programs (Genetics Computer Group, Madison, Wis. 53711)

According to certain methods for diagnosing a chronological life span or susceptibility to a chronological life span, hybridization methods, such as Southern analysis, Northern analysis, or in situ hybridizations, can be used (see Ausubel et al., supra). For example, a sample (e.g., a sample comprising genomic DNA, RNA, or cDNA), is obtained from a subject suspected of being susceptible to or having a chronological life span. The DNA, RNA, or cDNA sample is then examined to determine whether a polymorphic variant in a coding or noncoding portion of a gene set forth in Table 2, or a polymorphic variant in a genomic region linked to a coding or noncoding portion of a gene encoding as set forth in Table 2 is present. The presence of the polymorphic variant can be indicated by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant, e.g., a polymorphic variant indicative of susceptibility to a chronological life span.

In order to diagnose susceptibility to a chronological life span, a hybridization sample is formed by contacting the sample with at least one nucleic acid probe. The probe is typically a nucleic acid probe (which can be labeled, e.g., with a radioactive, fluorescent, or enzymatic label or tag) capable of hybridizing to mRNA, genomic DNA, and/or cDNA sequences encompassing detecting a polymorphic variant in a coding or noncoding portion of a gene set forth in Table 2, or a polymorphic variant in a genomic region linked to a coding or noncoding portion of a gene encoding as set forth in Table 2 is present. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA, cDNA, or genomic DNA.

The hybridization sample is maintained under conditions selected to allow specific hybridization of the nucleic acid probe to a region encompassing the polymorphic site. Specific hybridization can be performed under high stringency conditions or moderate stringency conditions, for example, as described above. In a particularly preferred embodiment, the hybridization conditions for specific hybridization are high stringency. In general, the probe can be perfectly complementary to the region to which it hybridizes, i.e., perfectly complementary to a region encompassing the polymorphic site when the site contains any particular polymorphic sequence. Multiple nucleic acid probes (e.g., multiple probes differing only at the polymorphic site, or multiple probes designed to detect polymorphic variants at multiple polymorphic sites) can be used concurrently in this method. Specific hybridization of any one of the nucleic acid probes is indicative of a polymorphic variant in a genomic region linked to a coding or noncoding portion of an expression profile gene set forth in Table 2 or fragment thereof, or detecting a polymorphic variant of a polymorphism in a genomic region linked to such a gene, and is thus diagnostic of susceptibility to a chronological life span.

Northern analysis can be performed using similar nucleic acid probes in order to detect a polymorphic variant of a polymorphism in a coding or noncoding portion of a gene selected from the group consisting of an expression profile gene set forth in Table 2 or fragment thereof, or detecting a polymorphic variant of a polymorphism in a genomic region linked to such a gene. See, e.g., Ausubel et al., supra.

According to certain embodiments of the invention, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker (see, for example, Nielsen, P. E. et al., 1994, Bioconjugate Chemistry 5 American Chemical Society, p. 1 (1994). The PNA probe can be designed to specifically hybridize to a nucleic acid comprising a polymorphic variant conferring susceptibility to or indicative of the presence of a chronological life span.

According to another method, restriction digest analysis can be used to detect the existence of a polymorphic variant of a polymorphism, if alternate polymorphic variants of the polymorphism result in the creation or elimination of a restriction site. A sample containing genomic DNA is obtained from the individual. Polymerase chain reaction (PCR) can be used to amplify a region comprising the polymorphic site, and restriction fragment length polymorphism analysis is conducted (see, e.g., Ausubel et al., supra). The digestion pattern of the relevant DNA fragment indicates the presence or absence of a particular polymorphic variant of the polymorphism and is therefore indicative of the presence or absence of susceptibility to a chronological life span.

Sequence analysis can also be used to detect specific polymorphic variants. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined.

Allele-specific oligonucleotides can also be used to detect the presence of a polymorphic variant, e.g., through the use of dot-blot hybridization of amplified oligonucleotides with allele-specific oligonucleotide (ASO) probes (see, for example, Saiki, R. et al., 1986, Nature 324:163-166). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains a polymorphism, e.g., a polymorphism associated with a susceptibility to a chronological life span. An allele-specific oligonucleotide probe that is specific for particular a polymorphism can be prepared, using standard methods (see Ausubel et al., supra).

To determine which of multiple polymorphic variants is present in a subject, a sample comprising DNA is obtained from the individual. PCR can be used to amplify a portion encompassing the polymorphic site. DNA containing the amplified portion can be dot-blotted, using standard methods, and the blot contacted with the oligonucleotide probe. The presence of specific hybridization of the probe to the DNA is then detected. Specific hybridization of an allele-specific oligonucleotide probe (specific for a polymorphic variant indicative of susceptibility to a chronological life span) to DNA from the subject is indicative of susceptibility to a chronological life span.

According to another embodiment of the invention, arrays of oligonucleotide probes that are complementary to nucleic acid portions from a subject can be used to identify polymorphisms. Biochips as described herein can be used.

The array typically includes oligonucleotide probes capable of specifically hybridizing to different polymorphic variants. According to the method, a nucleic acid of interest, e.g., a nucleic acid encompassing a polymorphic site, (which is typically amplified) is hybridized with the array and scanned. Hybridization and scanning are generally carried out according to standard methods. See, e.g., Published PCT Application Nos. WO 92/10092 and WO 95/11995, and U.S. Pat. No. 5,424,186. After hybridization and washing, the array is scanned to determine the position on the array to which the nucleic acid hybridizes. The hybridization data obtained from the scan is typically in the form of fluorescence intensities as a function of location on the array.

Arrays can include multiple detection blocks (i.e., multiple groups of probes designed for detection of particular polymorphisms). Such arrays can be used to analyze multiple different polymorphisms. Detection blocks can be grouped within a single array or in multiple, separate arrays so that varying conditions (e.g., conditions optimized for particular polymorphisms) can be used during the hybridization. For example, it can be desirable to provide for the detection of those polymorphisms that fall within G-C rich stretches of a genomic sequence, separately from those falling in A-T rich segments.

Additional description of use of oligonucleotide arrays for detection of polymorphisms can be found, for example, in U.S. Pat. Nos. 5,858,659 and 5,837,832. In addition, to oligonucleotide arrays, cDNA arrays can be used similarly in certain embodiments of the invention.

Other methods of nucleic acid analysis can be used to detect polymorphisms and/or polymorphic variants. Such methods include, e.g., direct manual sequencing (Church and Gilbert, Proc. Natl. Acad. Sci. USA 81: 1991-1995, 1988; Sanger et al., Proc. Natl. Acad. Sci. USA 74: 5463-5467, 1977; Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield et al., Proc. Natl. Acad. Sci. USA 86: 232-236, 1991), mobility shift analysis (Orita et al., Proc. Natl. Acad. Sci. USA 86: 2766-2770, 1989), restriction enzyme analysis (Flavell et al., Cell 15: 25, 1978; Geever et al., Proc. Natl. Acad. Sci. USA 78: 5081, 1981); heteroduplex analysis; chemical mismatch cleavage (CMC) (Cotton et al., Proc. Natl. Acad. Sci. USA 85: 4397-4401, 1985; RNase protection assays (Myers et al., Science 230: 1242, 1985); use of polypeptides that recognize nucleotide mismatches, e.g., E. coli mutS protein; allele-specific PCR.

In certain embodiments of the invention fluorescence polarization template-directed dye-terminator incorporation (FP-TDI) is used to determine which of multiple polymorphic variants of a polymorphism is present in a subject. This method is based on template-directed primer extension and detection by fluorescence polarization. According to this method, amplified genomic DNA containing a polymorphic site is incubated with oligonucleotide primers (designed to hybridize to the DNA template adjacent to the polymorphic site) in the presence of allele-specific dye-labeled dideoxyribonucleoside triphosphates and a commercially available modified Taq DNA polymerase. The primer is extended by the dye-terminator specific for the allele present on the template, increasing 10-fold the molecular weight of the fluorophore. At the end of the reaction, the fluorescence polarization of the two dye-terminators in the reaction mixture are analyzed directly without separation or purification. This homogeneous DNA diagnostic method has been shown to be highly sensitive and specific and is suitable for automated genotyping of large number of samples. (Chen et al., Genome Research 9: 492-498, 1999). Note that rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

Real-time pyrophosphate DNA sequencing is yet another approach to detection of polymorphisms and polymorphic variants. (Alderborn et al., Genome Research 10: 1249-1258, 2000). Additional methods include, for example, PCR amplification in combination with denaturing high performance liquid chromatography (dHPLC) (Underhill et al., Genome Research 7: 996-1005, 1997).

In general, it will be of interest to determine the genotype of a subject with respect to both copies of the polymorphic site present in the genome. For example, the complete genotype can be characterized as −/−, as −/+, or as +/+, where a minus sign indicates the presence of the reference or wild type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means above can be used to determine the genotype of a subject with respect to one or both copies of the polymorphism present in the subject's genome.

According to certain embodiments of the invention it is preferable to employ methods that can detect the presence of multiple polymorphic variants (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel can also be performed so as to detect the presence of multiple polymorphic variants (e.g., polymorphic variants at a plurality of polymorphic sites) in parallel or substantially simultaneously according to certain embodiments of the invention.

The invention provides a database comprising a list of polymorphic sequences stored on a computer-readable medium, wherein the polymorphic sequences occur in a coding or noncoding portion of a gene set forth in Table 2 or fragment thereof, or in a genomic region linked to such a gene, or in a genomic region linked to such a gene, and wherein the list is largely or entirely limited to polymorphisms have been identified as useful in performing genetic diagnosis of a chronological life span or susceptibility to a chronological life span, or for performing genetic studies of a chronological life span or susceptibility to a chronological life span.

### 43. Kits

The invention provides kits comprising the compositions, e.g., the differentially expressed protein, agonist or antagonist of the present invention or their homologs and are useful tools for examining expression and regulation of, for example, the genes as disclosed herein. Reagents that specifically hybridize to nucleic acids encoding differentially expressed proteins of the invention (including probes and primers of the differentially expressed proteins), and reagents that specifically bind to the differentially expressed proteins, e.g., antibodies, are used to examine expression and regulation.

Also within the scope of the invention are kits comprising the compositions (e.g., monoclonal antibodies, human sequence antibodies, human antibodies, multispecific and bispecific molecules, nucleic acid compositions, e.g., antisense oligonucleotides, double stranded RNA oligonucleotides (RNAi), or DNA oligonucleotides (vectors) containing nucleotide sequences encoding for the transcription of shRNA molecules) of the invention and instructions for use. The kit can further contain a least one additional reagent, or one or more additional human antibodies of the invention (e.g., a human antibody having a complementary activity which binds to an epitope in the antigen distinct from the first human antibody).

Nucleic acid assays for the presence of differentially expressed proteins in a sample include numerous techniques are known to those skilled in the art, such as Southern analysis, northern analysis, dot blots, RNase protection, S1 analysis, amplification techniques such as PCR and LCR, high density oligonucleotide array analysis, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques 4: 230-250, 1986; Haase et al., Methods in Virology 7: 189-226, 1984; and Nucleic Acid Hybridization: A Practical Approach (Hames et al., eds. 1987). In addition, a differentially expressed protein can be detected with the various immunoassay techniques described above. The test sample is typically compared to both a positive control (e.g., a sample expressing recombinant differentially expressed protein) and a negative control.

The present invention also provides for kits for screening drug candidates for treatment of chronological life span diseases or disorders or diseases or disorders associated with aging such as various types of cancers, diabetes mellitus, cataracts, heart diseases, and neurodegenerative diseases, such as Alzheimer's disease, Pick's disease, Huntington's disease, Parkinson's disease, adult onset myotonic dystrophy, multiple sclerosis, and adult onset leukodystrophy disease. Such kits can be prepared from readily available materials and reagents. For example, such kits can comprise any one or more of the following materials: the differentially expressed proteins, agonists, or antagonists of the present invention, pharmaceutical compositions that can modulate, or modify, the function of the identified genes and gene products, reaction tubes, and instructions for testing the activities of differentially expressed genes. A wide variety of kits and components can be prepared according to the present invention, depending upon the intended user of the kit and the particular needs of the user. For example, the kit can be tailored for in vitro or in vivo assays for measuring the activity of drug candidates for treatment of chronological life span diseases or disorders or related to diseases or conditions associated with aging as described herein.

The invention further provides kits comprising probe arrays as described above. The invention further provides oligonucleotide arrays comprising one or more of the inventive probes described above. In particular, the invention provides an oligonucleotide array comprising oligonucleotide probes that are able to detect polymorphic variants of the genes defined and disclosed herein. In a preferred embodiment the genes are defined in Table 1 or in Table 2. Such arrays can be provided in the form of kits for diagnostic and/or research purposes. Kits can include any of the components mentioned above, in addition to further components specific for hybridization and processing of oligonucleotide arrays. Appropriate software (i.e., computer-readable instructions stored on a computer-readable medium) for analyzing the results obtained by scanning the arrays can be provided by the invention. Such software can, for example, provide the user with an indication of the genotype of a sample and/or provide an assessment of the degree of susceptibility of the subject to chronological life span diseases or disorders or related to diseases or conditions associated with aging as described herein. According to certain embodiments of the invention, the kits are manufactured in accordance with good manufacturing practices (GMP) as required for FDA-approved diagnostic kits.

Optional additional components of the kit include, for example, other restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kits of the present invention also contain instructions for carrying out the methods.

The invention further provides compositions, kits and integrated systems for practicing the assays described herein. For example, an assay composition having a source of cells. Additional assay components as described above are also provided. For instance, a solid support or substrate in which the assays can be carried out can also be included. Such solid supports include membranes (e.g., nitrocellulose or nylon), a microliter dish (e.g., PVC, polypropylene, or polystyrene), a test tube (glass or plastic), a dipstick (e.g., glass, PVC, polypropylene, polystyrene, latex, and the like), a microcentrifuge tube, or a glass, silica, plastic, metallic or polymer bead or other substrate such as paper. Most commonly, the assay will use 96, 384 or 1536 well microtiter plates.

The kits can include any of the compositions noted above, and optionally further include additional components such as instructions to practice a high throughput method of screening for chronological life span modulators, one or more containers or compartments (e.g., to hold the cells, test agents, controls, dyes, and the like), a control activity modulator, a robotic armature for mixing kit components, and the like.

The invention also provides integrated systems for high throughput screening of potential modulators of chronological life span extension. Such systems typically include a robotic armature which transfers fluid from a source to a destination, a controller which controls the robotic armature, a label detector, a data storage unit which records label detection, and an assay component such as a microtiter dish.

A number of well-known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. Any of the assays for compounds that modulate activity, as described herein, are amenable to high throughput screening. High throughput screening systems are commercially available (see, e.g., Zymark Corp. (Hopkinton, Mass.); Air Technical Industries (Mentor, Ohio); Beckman Instruments, Inc. (Fullerton, Calif.); Precision Systems, Inc., (Natick, Mass.), and the like). Such systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for the various high throughput systems.

Optical images viewed (and, optionally, recorded) by a camera or other recording device (e.g., a photodiode and data storage device) are optionally further processed in any of the embodiments described herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS®, OS2® WINDOWS®, WINDOWS NT® or WINDOWS95® based machines), MACINTOSH®, or UNIX based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

The invention will be further described with reference to the following examples; however, it is to be understood that the invention is not limited to such examples.

### Exemplary Embodiments

Research Design and Methods

Experimental Approach. In order to measure the CLS of yeast cells, it is necessary to (1) maintain a culture of cells in a non-dividing state for at least several weeks and (2) quantitatively measure the viability of cells in the culture over time. CLS assays have been previously performed by the continuous culturing of cells in 5-25 mL of liquid (synthetic complete media, rich media, or water), either in culture tubes on a rotating drum or in flasks on a platform shaker. Each tube or flask contains one strain, and the viability of each strain is measured over time by serial dilution and plating onto rich media for determination of CFUs. These methods work well for a relatively small number of strains (<20) assayed in parallel; however, it is not feasible to perform genome-wide analysis of more than 4000 strains in this manner. Therefore, we have developed technology more suitable for the simultaneous quantitative measurement of CLS for several thousand strains. Several major improvements over the traditional CLS assays have been made, including (1) measurement of viability (relative to a reference) by optical density (OD), (2) long-term culturing of yeast cells in sub-milliliter volumes using 96-well microtiter plates, and (3) utilization of robotic systems for automated dilution and cell transfer.

Determination of viability by optical density. Spectrophotometric methods for the quantitative measurement of cell density of a yeast culture are well established. Over a range (typically ˜0.1 to ˜0.8), OD₆₆₀ varies linearly with cell density. As described herein, a highly quantitative method for determining the number of viable cells present in a small volume (˜2 μL) of solution has been developed which has taken advantage of this property. As shown in FIG. 1, the method is accomplished by (1) diluting a small volume of cells into a large volume of growth media, (2) incubating the cells for a fixed period of time under defined conditions favorable for growth, and (3) measuring the OD of the large culture after the growth period. Assuming that measurement occurs within the range where OD varies linearly with cell density, the final OD is proportional to the number of viable cells present in the initial small volume. This method is optimally suited for high throughput analysis using automated systems. For example, the Biomek FX Laboratory Automation Robot (Beckman Coulter) is capable of simultaneously transferring a precise volume of liquid from each well of a 96-well plate into corresponding wells of a second 96-well plate. The transfer process is accurate and highly reproducible down to 1 μL.

There are at least two ways to calculate viability of cells in a CLS experiment for the assays: 1) after outgrowth, the OD of a specific well can be compared to the average OD for wells in an individual plate or group of plates, to determine relative viability of the cells in that specific well relative to the average; 2) The OD of a specific well after outgrowth can be compared to the OD measured for that same well at the first time point taken during the experiment. For example if a specific well has an OD of 0.600 after outgrowth at the first time point, we designate that as 100% viability. This measurement is used as a standard for that well for the rest of the experiment. If at the second time point that specific well has an OD of 0.300, we can calculate 50% viability at the second time point.

In order to validate this method, a series of heat shock sensitivity experiments were carried out in which viability after incubation at high temperature was measured by both the standard CFU assay and by the claimed method for the determination of viability by OD after outgrowth (DVOD). BY4742 cells were grown to stationary phase overnight, aliquoted into separate tubes, and subjected to heat shock at 55° C. for 0, 5, 10, or 15 minutes. Heat-shocked or control cells were then transferred to individual wells of a 96-well plate. DVOD was carried out by inoculating 2 μL from each well into 200 μL of rich media in the corresponding wells of three additional 96-well plates using the Biomek FX robot. The plates were then incubated at 30° C. to allow outgrowth, and OD₆₆₀ was measured in each well using a Victor plate reader (Wallace) over the course of 48 hours. In order to calculate survival, the OD₆₆₀ for wells containing untreated cells was defined as 100%. Percent viability after heat-shock was calculated by dividing the OD₆₆₀ for wells containing heat-shocked cells by the OD₆₆₀ for wells containing untreated cells. Viability as determined by DVOD was highly correlated with viability determined in parallel by CFUs (FIG. 2).

Life Span Screening of the Genome-Wide Deletion Collection. We set about applying this method to the yeast deletion collection, which is an array of ˜4,800 yeast strains, each with a single gene deleted. We were able to quantify the life span of every strain in the deletion collection by measuring viability over serial time points. The viability (as measured by CLSOD) of each deletion strain was ranked relative to the average viability of the entire set. This analysis allowed us to assign relative life span values for the entire deletion collection.

Identification of Long-lived Mutants, and Retesting. We choose to focus on our top 90 long-lived deletion strains and retested their life spans directly by comparing them to the wild type (parental) strain. Of the 52 that retested for life span extension, we observed that 16 of these strains were deleted for genes involved in the Tor pathway, including its founding member TOR1 (Table 1; see Table 2 below):

TABLE 1

| | |
|---|---|
| gln3 | nitrogen regulated Transcription factor (regulated by Tor) |
| lys12 | lysine biosynthesis |
| ygl007w | molecular_function unknown (implicated in Tor signaling) |
| Mep2 | ammonium transporter activity |
| Rpp2a | structural constituent of ribosome |
| Mep3 | ammonium transporter activity |
| tef4 | ribosomal stability |
| gtr2 | GTPase (implicated in Tor signaling) |
| ygr054w | translation initiation factor activity |
| rtg2 | intracellular signaling cascade |
| dal80 | nitrogen regulated Transcription factor (regulated by Tor) |
| Agp1 | amino acid transport |
| gtr1 | GTPase (implicated in Tor signaling) |
| ybr077c | molecular_function unknown (implicated in Tor signaling) |
| rps25a | structural constituent of ribosome |
| tor1 | nutrient sensing |

Exemplary Orthologous Sequences Identified by Database Search. Approximately 16 unique open reading frames (“ORFs”) of yeast sequences are identified from the variants classified by methods of the present invention. For each yeast sequence from the genes listed in Table 1 as conferring “long-lived” or chronological life-span-regulating, the amino-acid sequence of the corresponding protein is used as a “query sequence” to perform a search against sequences deposited within various public databases to identify evolutionarily-related sequences. Coding sequences for each yeast ORF are obtained from a genomic database for Saccharomyces cerevisiae.

A BLAST search of the NCBI database resulted in the identification of various mammalian orthologs that are related to the ORFs listed in Table 1. Table 2 lists exemplary sets of conserved orthologs that correspond to the yeast homolog. For each identified yeast ORF, the respective percent identities, percent similarities, and E values are shown. Default parameters were used to perform the search. For orthologs, accession numbers relating to the NCBI database for a polypeptide sequence are provided below.

For the present invention, an ortholog is defined as a homologous molecule or sequence having life-span-regulating activity and a sequence identity of at least 20%, 25%, 30%, 35%, 40%, 45%, 50%, 55%, 60%, 65%, 70%, 75%, 80%, 85%, 90%, and 95%. Alternatively, an ortholog is defined as a homologous molecule or sequence having life-span-regulating activity and a sequence similarity of at least about 40%, 45%, 50%, 55%, 60%, 65%, 70%, 75%, 80%, 85%, 90%, and 95%.

TABLE 2

| | | | | | | | |
|---|---|---|---|---|---|---|---|
| | | | Match | % | % | High | E |
| Gene | GenBank | Synonyms/Description | Length | Iden | Sim | Score | Val |

GLN3 - AAB64575
GLN3 - blast vs. human

| | | | | | | | |
|---|---|---|---|---|---|---|---|
| GATA6 | NP_005248.2 | GATA6 GATA-binding protein 6, a member of GATA | 81 | 41% | 58% | 172 | 6e−13 |
| | | family of zinc-finger transcription factors, involved in | | | | | |
| | | the differentiation of vascular smooth muscle cells, | | | | | |
| | | implicated in cell proliferation and development; may be | | | | | |
| | | linked to sex cord-derived ovarian tumors | | | | | |
| GATA4 | NP_002043.2 | GATA4 GATA binding protein 4, a putative zinc-finger | 173 | 27% | 51% | 185 | 8e−13 |
| | | transcription factor that may play a role in heart and gut | | | | | |
| | | development, overproduction is detected in esophageal | | | | | |
| | | adenocarcinomas and adrenocortical carcinomas | | | | | |
| GATA5 | NP_536721.1 | GATA5; bB379O24.1 GATA binding protein 5, a | 89 | 45% | 58% | 199 | 1e−11 |
| | | putative transcriptional activator that may function in | | | | | |
| | | heart development | | | | | |
| TRPS1 | NP_054831.1 | TRPS1; GC79 Trichorhinophalangeal syndrome I, | 102 | 35% | 52% | 167 | 1e−11 |
| | | contains a GATA-type zinc finger and an Ikaros family | | | | | |
| | | domain, may act as a transcriptional repressor; mutation | | | | | |
| | | of the corresponding gene is associated with | | | | | |
| | | trichorhinophalangeal syndrome types I, II, and III | | | | | |
| GATA1 | NP_002040.1 | GATA1; ERYF1; (GF1); NFE1; GF-1; (NF-E1) GATA- | 62 | 47% | 65% | 158 | 7e−11 |
| | | binding protein 1, member of the GATA family of | | | | | |
| | | transcription factors that participates in erythropoiesis | | | | | |
| | | and is associated with Down syndrome-associated acute | | | | | |
| | | megakaryoblastic leukemia and transient | | | | | |
| | | myeloproliferative disorder upon gene mutation | | | | | |
| GATA2 | NP_116027.2 | GATA2; NFE1B; MGC2306 GATA binding protein 2, | 79 | 42% | 57% | 151 | 6e−10 |
| | | transcriptional activator, regulates expression of | | | | | |
| | | erythroid-specific genes (perhaps in conjunction with | | | | | |
| | | GATA1), abnormal expression may play a role in | | | | | |
| | | leukemia; rat Gata2 is downregulated in Pneumocystis | | | | | |
| | | carinii infections | | | | | |
| GATA3 | NP_001002295.1 | | | | | | |

Compared with M. musculus protein sequences (Documentation)

| | | | | | | | |
|---|---|---|---|---|---|---|---|
| Gata6 | NP_034388.2 | Gata6; Mm.33783; GATA-6 GATA-binding protein 6, a | 91 | 40% | 57% | 177 | 2e−13 |
| | | member of GATA family of Zinc-finger transcription factors | | | | | |
| | | that may play a critical role during differentiation and | | | | | |
| | | development and cell cycle arrest; human GATA6 may be | | | | | |
| | | linked to sex cord derived ovarian tumors | | | | | |
| Gata4 | NP_032118.2 | Gata4; Mm.1428; Gata-4 GATA-binding protein 4, zinc- | 173 | 28% | 51% | 188 | 1e−12 |
| | | finger transcription factor that plays a role in sex | | | | | |
| | | differentiation and heart and gut development; overproduction | | | | | |
| | | of human GATA4 is detected in esophageal adenocarcinomas | | | | | |
| | | and adrenocortical carcinomas | | | | | |
| Trps1 | NP_114389.1 | Trps1; D15Ertd586e; MGC46754 Trichorhinophalangeal | 102 | 35% | 51% | 166 | 1e−11 |
| | | syndrome I (human), has a GATA-type zinc finger and an | | | | | |
| | | Ikaros family domain, represses transcription mediated by | | | | | |
| | | other GATA factors; mutation of the human TRPS1 gene is | | | | | |
| | | linked to trichorhinophalangeal syndrome types I, II, and III | | | | | |
| Gata5 | NP_032119.1 | Gata5; Mm.2527; GATA-5 GATA binding protein 5, a | 82 | 43% | 60% | 175 | 4e−11 |
| | | transcriptional activator involved in endothelial cell | | | | | |
| | | differentiation, embryonic urogenital system development and | | | | | |
| | | possibly heart and lung development, may function in | | | | | |
| | | development of smooth muscle cell diversity | | | | | |
| Gata1 | NP_032115.1 | Gata1; Gata-1; Gf-1 GATA-binding protein 1, member of the | 72 | 43% | 64% | 157 | 1e−10 |
| | | GATA family of transcription factors that acts in | | | | | |
| | | erythropoiesis and regulates Sertoli cell gene expression; | | | | | |
| | | human GATA1 is is associated with acute megakaryoblastic | | | | | |
| | | leukemia and transient myeloproliferative disorder | | | | | |
| Gata3 | NP_032117.1 | Gata3; Mm.606; Gata-3 GATA-binding protein 3, zinc-finger | 79 | 42% | 58% | 153 | 3e−10 |
| | | transcription factor, involved in T-cell differentiation, defense | | | | | |
| | | response, neurogenesis, and cell proliferation, may be | | | | | |
| | | involved in asthma; human GATA3 may be involved in breast | | | | | |
| | | cancer, HDR syndrome, HIV-1 activation | | | | | |
| Gata2 | NP_032116.3 | Gata2; Mm.1391; Gata-2 GATA binding protein 2, | 79 | 42% | 58% | 154 | 4e−09 |
| | | transcriptional activator, acts in hematopoiesis and urogenital | | | | | |
| | | development, potentiates generation of V2 interneurons; | | | | | |
| | | human GATA2 mis-expression may play a role in leukemia, | | | | | |
| | | rat Gata2 is downregulated in P. carinii infections | | | | | |

Compared with C. elegans protein sequences (Documentation)

| | | | | | | | |
|---|---|---|---|---|---|---|---|
| Y48A5B.C | | Y48A5B.C Protein with strong similarity to C. elegans | 230 | 23% | 41% | 177 | 1e−12 |
| | | ELT-6, which is required for embryonic development | | | | | |
| | | and functions to repress vulval cell fusion and regulate | | | | | |
| | | cell fate determination, contains a GATA-type zinc | | | | | |
| | | finger domain | | | | | |
| elt-6 | AAC68957.3 | elt-6; F52C12.5 Erythroid-like-transcription factor 6, | 201 | 25% | 45% | 176 | 1e−12 |
| | | protein required for embryonic development, functions to | | | | | |
| | | repress vulval cell fusion and regulate cell fate | | | | | |
| | | determination | | | | | |
| elt-1 | CAA92494.1 | elt-1; W09C2.1 Erythroid-like transcription factor 1, | 304 | 25% | 42% | 194 | 9e−12 |
| | | GATA transcription factor involved in embryogenesis, | | | | | |
| | | regulation of movement, egg-laying, and adult life span | | | | | |
| | | determination, activates LIN-26 in the hypodermis, may | | | | | |
| | | act to specify the major hypodermal cell fate | | | | | |
| elt-2 | CAA90029.2 | elt-2; C33D3.1 Erythroid-like transcription factor 2, | 138 | 29% | 51% | 158 | 4e−10 |
| | | GATA-type zinc finger DNA-binding factor involved in | | | | | |
| | | development of the gut, larval growth, reproduction, | | | | | |
| | | regulation of movement, and osmoregulation | | | | | |
| egl-18 | AAD36952.2 | egl-18; elt-5; F55A8.1 Egg-laying abnormal 18, protein | 248 | 25% | 42% | 163 | 7e−10 |
| | | required for embryonic development, functions to repress | | | | | |
| | | vulval cell fusion and regulate cell fate determination | | | | | |
| elt-3 | | elt-3; K02B9.4 Erythroid-like transcription factor family | 190 | 31% | 44% | 157 | 1e−09 |
| | | 3, GATA-binding factor that is required for hypodermal | | | | | |
| | | cell differentiation | | | | | |
| elt-4 | CAD44111.1 | elt-4; C39B10.6 Erythroid-like transcription factor 4, | 70 | 41% | 57% | 136 | 5e−08 |
| | | small GATA-type zinc finger domain-containing protein | | | | | |
| | | that binds DNA weakly and non-specifically | | | | | |
| end-1 | CAB04513.1 | end-1; F58E10.2 Endoderm specification 1, GATA | 80 | 35% | 50% | 127 | 4e−07 |
| | | transcription factor expressed in zygotes and required for | | | | | |
| | | development of the gut | | | | | |
| C18G1.2 | AAC17756.1 | C18G1.2 Protein containing a GATA-type zinc finger | 55 | 45% | 60% | 120 | 3e−06 |
| | | domain, has low similarity to a region of C. elegans | | | | | |
| | | ELT-3, which is a GATA-binding factor required for | | | | | |
| | | hypodermal cell differentiation | | | | | |
| end-3 | CAB04516.1 | end-3; F58E10.5 Endoderm determining 3, protein | 263 | 24% | 36% | 126 | 9e−06 |
| | | involved in differentiation of intestinal cells, | | | | | |
| | | transcriptional target of the repressor POP-1 and the | | | | | |
| | | activator MED-1 in the EMS lineage | | | | | |
| med-2 | AAK93857.1 | med-2; K04C2.6 GATA-type transcription factor | 166 | 27% | 43% | 123 | 9e−06 |
| med-1 | CAA92204.2 | med-1; T24D3.1 Mesendodem specification 1, GATA- | 168 | 28% | 43% | 128 | 3e−04 |
| | | type transcription factor | | | | | |

LYS12 - CAA86700
LYS12 blast vs human -

| | | | | | | | |
|---|---|---|---|---|---|---|---|
| IDH3A | NP_005521.1 | IDH3A; IDHalpha Isocitrate dehydrogenase 3 (NAD+) | 351 | 37% | 55% | 522 | 6e−53 |
| | | alpha, catalytic subunit of the mitochondrial enzyme that | | | | | |
| | | catalyzes the oxidative decarboxylation of isocitrate to form | | | | | |
| | | alpha-ketoglutarate in the tricarboxylic acid cycle | | | | | |
| IDH3G | NP_004126.1 | IDH3G; H-IDH_gamma; IDHgamma; H-IDHG NAD(+)- | 351 | 33% | 53% | 426 | 2e−41 |
| | | dependent isocitrate dehydrogenase gamma subunit, | | | | | |
| | | catalyzes the oxidative decarboxylation of isocitrate into | | | | | |
| | | alpha-ketoglutarate in the TCA cycle | | | | | |
| IDH3B | NP_008830.2 | IDH3B; IDHbeta; H-IDHB; MGC903; FLJ11043 Isocitrate | 383 | 31% | 50% | 414 | 9e−36 |
| | | dehydrogenase 3 (NAD+) beta, a putative regulatory subunit | | | | | |
| | | of mitochondrial isocitrate dehydrogenase, which catalyzes | | | | | |
| | | the oxidative decarboxylation of isocitrate to form alpha- | | | | | |
| | | ketoglutarate in the tricarboxylic acid cycle | | | | | |
| IDH2 | NP_002159.2 | IDH2; (IDH); (IDP); IDHM; ICD-M; mNADP-IDH | 174 | 29% | 47% | 129 | 3e−07 |
| | | Isocitrate dehydrogenase 2 (NADP+) mitochondrial, | | | | | |
| | | catalyzes the oxidative decarboxylation of isocitrate to form | | | | | |
| | | alpha-ketoglutarate; gene variant of mouse Idh2 is detected | | | | | |
| | | in the epileptic strain E1 | | | | | |
| IDH1 | NP_005887.2 | IDH1; (IDH); PICD; (IDP) Cytosolic NADP(+)-dependent | 166 | 29% | 49% | 122 | 2e−06 |
| | | isocitrate dehydrogenase, catalyzes the oxidative | | | | | |
| | | decarboxylation of isocitrate into alpha-ketoglutarate, the | | | | | |
| | | key rate-limiting step of the citric acid (tricarboxylic) cycle | | | | | |

Compared with M. musculus protein sequences (Documentation)

| | | | | | | | |
|---|---|---|---|---|---|---|---|
| Idh3a | NP_083849.1 | Idh3a; 1500012E04Rik; 1110003P10Rik Protein | 351 | 37% | 55% | 517 | 2e−52 |
| | | with very strong similarity to isocitrate | | | | | |
| | | dehydrogenase 3 (NAD+) alpha (human IDH3A), | | | | | |
| | | which is the catalytic subunit of a key enzyme of | | | | | |
| | | the tricarboxylic acid cycle, contains an isocitrate or | | | | | |
| | | isopropylmalate dehydrogenase domain | | | | | |
| Idh3g | NP_032349.1 | Idh3g; Mm.14825 NAD(+)-dependent isocitrate | 351 | 33% | 53% | 426 | 1e−41 |
| | | dehydrogenase gamma subunit, catalyzes the | | | | | |
| | | oxidative decarboxylation of isocitrate into alpha- | | | | | |
| | | ketoglutarate, in the TCA cycle | | | | | |
| Idh3b | NP_570954.1 | Idh3b; Mm.29590 Protein with high similarity to | 354 | 32% | 51% | 413 | 4e−40 |
| | | NAD(+)-dependent isocitrate dehydrogenase | | | | | |
| | | gamma subunit (human IDH3G), which catalyzes | | | | | |
| | | the oxidative decarboxylation of isocitrate into | | | | | |
| | | alpha-ketoglutarate, contains an isocitrate or | | | | | |
| | | isopropylmalate dehydrogenase domain | | | | | |
| 4933405O20Rik | NP_766489.1 | 4933405O20Rik; 4933405O20 Protein with high | 352 | 32% | 52% | 409 | 1e−39 |
| | | similarity to NAD(+)-dependent isocitrate | | | | | |
| | | dehydrogenase gamma subunit (human IDH3G), | | | | | |
| | | which catalyzes the oxidative decarboxylation of | | | | | |
| | | isocitrate into alpha-ketoglutarate, contains an | | | | | |
| | | isocitrate or isopropylmalate dehydrogenase | | | | | |
| | | domain | | | | | |
| Idh2 | NP_766599.1 | Idh2; Mm.2966; mNADP-IDH; Idh-2; IDPm; | 174 | 29% | 47% | 126 | 1e−06 |
| | | E430004F23 Isocitrate dehydrogenase 2 (NADP+) | | | | | |
| | | mitochondrial, catalyzes the decarboxylation of | | | | | |
| | | isocitrate to form alpha-ketoglutarate, plays a role | | | | | |
| | | in cellular defense against reactive oxygen species, | | | | | |
| | | a variant form is detected in the epileptic mutant | | | | | |
| | | strain E1 | | | | | |
| Idh1 | NP_034627.2 | Idh1; Mm.9925; Id-1; Idh-1; E030024J03Rik; | 128 | 30% | 52% | 120 | 2e−06 |
| | | IDPc; MGC115782 Cytosolic NADP(+)-dependent | | | | | |
| | | isocitrate dehydrogenase, catalyzes the oxidative | | | | | |
| | | decarboxylation of isocitrate into alpha- | | | | | |
| | | ketoglutarate, the key rate-limiting step of the citric | | | | | |
| | | acid (tricarboxylic) cycle | | | | | |

Compared with C. elegans protein sequences (Documentation)

| | | | | | | | |
|---|---|---|---|---|---|---|---|
| F43G9.1 | CAB02111.2 | F43G9.1 Putative NAD+ isocitrate dehydrogenase that | 348 | 36% | 57% | 519 | 5e−53 |
| | | functions in embryogenesis and regulation of DNA | | | | | |
| | | transposition | | | | | |
| C37E2.1 | CAB02822.1 | C37E2.1 Protein with high similarity to NAD(+)- | 355 | 34% | 52% | 440 | 4e−43 |
| | | dependent isocitrate dehydrogenase gamma subunit | | | | | |
| | | (human IDH3G), which catalyzes the formation of alpha- | | | | | |
| | | ketoglutarate in the tricarboxylic acid cycle, contains an | | | | | |
| | | isocitrate or isopropylmalate dehydrogenase domain | | | | | |
| F35G12.2 | CAA86325.2 | F35G12.2 Putative NAD+-isocitrate dehydrogenase | 372 | 31% | 51% | 391 | 5e−37 |
| C30F12.7 | AAK85453.1 | C30F12.7 Protein with high similarity to C. elegans | 352 | 31% | 51% | 371 | 5e−35 |
| | | F35G12.2, which is involved in gametogenesis, larval | | | | | |
| | | development, and embryogenesis or morphogenesis, | | | | | |
| | | contains an isocitrate or isopropylmalate dehydrogenase | | | | | |
| | | domain | | | | | |
| C34F6.8 | CAB03943.1 | C34F6.8 Protein with high similarity to mouse Idh2, | 201 | 27% | 45% | 126 | 3e−06 |
| | | which is a mitochondrial isocitrate dehydrogenase, | | | | | |
| | | contains an isocitrate or isopropylmalate dehydrogenase | | | | | |
| | | domain | | | | | |
| F59B8.2 | CAA92778.1 | F59B8.2 Protein with high similarity to cytosolic | 180 | 30% | 45% | 123 | 4e−06 |
| | | NADP(+)-dependent isocitrate dehydrogenase (rat Idh1), | | | | | |
| | | which catalyzes the oxidative decarboxylation of isocitrate | | | | | |
| | | into alpha-ketoglutarate, contains an isocitrate or | | | | | |
| | | isopropylmalate dehydrogenase domain | | | | | |

MEP2 - CAA96025.1
MEP2 blast vs. human

| | | | | | | | |
|---|---|---|---|---|---|---|---|
| RHCG | NP_057405.1 | RHCG; RHGK; C15orf6; PDRC2 Rhesus blood group C | 299 | 24% | 39% | 149 | 7e−09 |
| | | glycoprotein, an ammonium transporter that functions in red | | | | | |
| | | blood cells, may also act as an epithelial transporter in | | | | | |
| | | kidney and testis, reduced expression is associated with | | | | | |
| | | development of esophageal squamous cell carcinoma | | | | | |
| RHBG | NP_065140.1 | RHBG Rhesus blood group B glycoprotein, a transmembrane | 238 | 22% | 40% | 125 | 3e−06 |
| | | protein that may function as an ammonium transporter | | | | | |
| RHAG | NP_000315.1 | RHAG; RH50A; RH2; Rh50; Rh50_GP Rhesus blood group- | 356 | 24% | 40% | 109 | 4e−04 |
| | | associated glycoprotein, a component of the Rh antigen, | | | | | |
| | | plays a role in the antigen transport to the cell surface, and | | | | | |
| | | may play a role in ammonium transport; mutation in the | | | | | |
| | | corresponding gene causes Rh deficiency and Rh-mod | | | | | |
| | | syndrome | | | | | |

Compared with M. musculus protein sequences (Documentation)

| | | | | | | | |
|---|---|---|---|---|---|---|---|
| Rhcg | NP_062773.1 | Rhcg; Mm.10909 Rhesus blood group C glycoprotein, may | 363 | 25% | 41% | 183 | 2e−12 |
| | | function as an epithelial transporter that helps maintain | | | | | |
| | | homeostasis in kidney and testis; reduced expression of human | | | | | |
| | | RHCG is associated with development of esophageal | | | | | |
| | | squamous cell carcinoma | | | | | |
| Rhbg | NP_067350.2 | Rhbg; Mm.103777 Rhesus blood group-associated B | 253 | 23% | 40% | 124 | 3e−06 |
| | | glycoprotein, a plasma membrane protein that may function as | | | | | |
| | | an ammonium transporter | | | | | |
| Rhag | NP_035399.1 | Rhag; Mm.12961; Rh50A; Rh50; CD241 Rhesus blood group- | 335 | 25% | 41% | 123 | 2e−05 |
| | | associated glycoprotein, a component of the Rh antigen; | | | | | |
| | | mutation in the human RHAG gene causes Rh deficiency and | | | | | |
| | | Rh-mod syndrome | | | | | |

Compared with C. elegans protein sequences (Documentation)

| | | | | | | | |
|---|---|---|---|---|---|---|---|
| C05E11.4 | AAA96191.1 | C05E11.4 Member of the ammonium transporter family | 467 | 27% | 44% | 350 | 1e−31 |
| | | of membrane transporters, has low similarity to S. cerevisiae | | | | | |
| | | Mep1p, which is an ammonia permease of high | | | | | |
| | | capacity and moderate affinity | | | | | |
| C05E11.5 | AAA96190.2 | C05E11.5 Member of the ammonium transporter family | 428 | 28% | 46% | 343 | 7e−31 |
| | | of membrane transporters, has low similarity to S. cerevisiae | | | | | |
| | | Mep3p, which is an ammonia permease of high | | | | | |
| | | capacity and low affinity | | | | | |
| M195.3 | CAA91293.2 | M195.3 Member of the ammonium transporter family of | 441 | 26% | 44% | 320 | 1e−28 |
| | | membrane transporters, has a region of low similarity to S. cerevisiae | | | | | |
| | | Mep2p, which is an ammonia permease of low | | | | | |
| | | capacity and high affinity | | | | | |
| F49E11.3 | CAA94345.1 | F49E11.3 Member of the ammonium transporter family of | 484 | 23% | 38% | 237 | 8e−19 |
| | | membrane transporters, has weak similarity to S. cerevisiae | | | | | |
| | | Mep2p, which is an ammonia permease of low | | | | | |
| | | capacity and high affinity | | | | | |
| rhr-1 | AAF97865.1 | rhr-1; F08F3.3 Member of the ammonium transporter | 326 | 24% | 39% | 132 | 2e−06 |
| | | family of membrane transporters, has moderate similarity | | | | | |
| | | to rhesus glycoprotein type C (human RHCG), which is an | | | | | |
| | | ammonium transporter that may be involved in cell | | | | | |
| | | growth and maintenance | | | | | |
| rhr-2 | AAF97864.1 | rhr-2; B0240.1 Member of the ammonium transporter | 325 | 24% | 41% | 119 | 5e−05 |
| | | family of membrane transporters, has moderate similarity | | | | | |
| | | to rhesus glycoprotein type C (human RHCG), which is an | | | | | |
| | | ammonium transporter | | | | | |

RPP2A - CAA99041.1
RPP2A blast vs. human -

| | | | | | | | |
|---|---|---|---|---|---|---|---|
| RPLP2 | NP_000995.1 | RPLP2; (P2); (RPP2); Hs.302588; D11S2243E Ribosomal | 115 | 53% | 73% | 271 | 9e−07 |
| | | protein large P2, an acidic phosphoprotein component of the | | | | | |
| | | large 60S ribosomal subunit, autoantibodies are associated | | | | | |
| | | with systemic lupus erythematosus, selectively upregulated | | | | | |
| | | in pancreatic cancer cell lines containing an activated K-Ras | | | | | |

Compared with M. musculus protein sequences (Documentation)

| | | | | | | | |
|---|---|---|---|---|---|---|---|
| Rplp2 | NP_080296.2 | Rplp2; 2700049I22Rik; Mm.14245 Protein with very strong | 115 | 53% | 72% | 267 | 3e−10 |
| | | similarity to ribosomal protein large P2 (human RPLP2), | | | | | |
| | | which is a component of the 60s ribosomal subunit that is | | | | | |
| | | autoantigenic in individuals with systemic lupus | | | | | |
| | | erythematosus, member of the 60s acidic ribosomal protein | | | | | |
| | | family | | | | | |

Compared with C. elegans protein sequences (Documentation)

| | | | | | | | |
|---|---|---|---|---|---|---|---|
| rpa-2 | CAB60595.1 | rpa-2; Y62E10A.D; Y62E10A.1 Acidic ribosomal subunit | 112 | 47% | 62% | 224 | 3e−06 |
| | | protein P2, protein involved with positive growth | | | | | |
| | | regulation | | | | | |
| C37A2.7 | AAB52450.2 | C37A2.7 Protein involved in positive growth regulation | 109 | 53% | 71% | 261 | 1e−05 |

MEP3 - AAB68278.1
MEP3 blast vs. human -

| | | | | | | | |
|---|---|---|---|---|---|---|---|
| RHBG | NP_065140.1 | RHBG Rhesus blood group B glycoprotein, a transmembrane | 238 | 27% | 39% | 123 | 4e−06 |
| | | protein that may function as an ammonium transporter | | | | | |
| RHAG | NP_000315.1 | RHAG; RH50A; RH2; Rh50; Rh50_GP Rhesus blood group- | 225 | 28% | 46% | 122 | 1e−05 |
| | | associated glycoprotein, a component of the Rh antigen, | | | | | |
| | | plays a role in the antigen transport to the cell surface, and | | | | | |
| | | may play a role in ammonium transport; mutation in the | | | | | |
| | | corresponding gene causes Rh deficiency and Rh-mod | | | | | |
| | | syndrome | | | | | |
| RHCG | NP_057405.1 | RHCG; RHGK; C15orf6; PDRC2 Rhesus blood group C | 313 | 24% | 40% | 120 | 3e−05 |
| | | glycoprotein, an ammonium transporter that functions in red | | | | | |
| | | blood cells, may also act as an epithelial transporter in | | | | | |
| | | kidney and testis, reduced expression is associated with | | | | | |
| | | development of esophageal squamous cell carcinoma | | | | | |

Compared with M. musculus protein sequences (Documentation)

| | | | | | | | |
|---|---|---|---|---|---|---|---|
| Rhcg | NP_062773.1 | Rhcg; Mm.10909 Rhesus blood group C glycoprotein, may | 328 | 25% | 38% | 129 | 1e−06 |
| | | function as an epithelial transporter that helps maintain | | | | | |
| | | homeostasis in kidney and testis; reduced expression of human | | | | | |
| | | RHCG is associated with development of esophageal | | | | | |
| | | squamous cell carcinoma | | | | | |
| Rhbg | NP_067350.2 | Rhbg; Mm.103777 Rhesus blood group-associated B | 193 | 26% | 40% | 113 | 6e−05 |
| | | glycoprotein, a plasma membrane protein that may function as | | | | | |
| | | an ammonium transporter | | | | | |
| Rhag | NP_035399.1 | Rhag; Mm.12961; Rh50A; Rh50; CD241 Rhesus blood group- | 223 | 26% | 43% | 111 | 1e−04 |
| | | associated glycoprotein, a component of the Rh antigen; | | | | | |
| | | mutation in the human RHAG gene causes Rh deficiency and | | | | | |
| | | Rh-mod syndrome | | | | | |

Compared with C. elegans protein sequences (Documentation)

| | | | | | | | |
|---|---|---|---|---|---|---|---|
| C05E11.4 | AAA96191.1 | C05E11.4 Member of the ammonium transporter family | 445 | 27% | 46% | 370 | 1e−34 |
| | | of membrane transporters, has low similarity to S. cerevisiae | | | | | |
| | | Mep1p, which is an ammonia permease of | | | | | |
| | | high capacity and moderate affinity | | | | | |
| C05E11.5 | AAA96190.2 | C05E11.5 Member of the ammonium transporter family | 443 | 28% | 45% | 362 | 2e−33 |
| | | of membrane transporters, has low similarity to S. cerevisiae | | | | | |
| | | Mep3p, which is an ammonia permease of | | | | | |
| | | high capacity and low affinity | | | | | |
| M195.3 | CAA91293.2 | M195.3 Member of the ammonium transporter family of | 413 | 25% | 45% | 293 | 2e−25 |
| | | membrane transporters, has a region of low similarity to | | | | | |
| | | S. cerevisiae Mep2p, which is an ammonia permease of | | | | | |
| | | low capacity and high affinity | | | | | |
| F49E11.3 | CAA94345.1 | F49E11.3 Member of the ammonium transporter family | 457 | 23% | 40% | 236 | 1e−18 |
| | | of membrane transporters, has weak similarity to S. cerevisiae | | | | | |
| | | Mep2p, which is an ammonia permease of | | | | | |
| | | low capacity and high affinity | | | | | |
| rhr-1 | AAF97865.1 | rhr-1; F08F3.3 Member of the ammonium transporter | 224 | 24% | 38% | 101 | 0.001 |
| | | family of membrane transporters, has moderate | | | | | |
| | | similarity to rhesus glycoprotein type C (human RHCG), | | | | | |
| | | which is an ammonium transporter that may be involved | | | | | |
| | | in cell growth and maintenance | | | | | |

TEF4 - CAA81919.1
TEF4 blast vs human -

| | | | | | | | |
|---|---|---|---|---|---|---|---|
| EEF1G | NP_001395.1 | EEF1G; EF1G Eukaryotic translation elongation factor 1 | 451 | 33% | 51% | 586 | 1e−49 |
| | | gamma, a putative translation elongation factor 1 (EF-1) | | | | | |
| | | complex subunit that binds cytoplasmic cysteinyl-tRNA | | | | | |
| | | synthetase and possibly EF-1 beta, upregulated in gastric | | | | | |
| | | and colorectal cancer | | | | | |

Compared with M. musculus protein sequences (Documentation)

| | | | | | | | |
|---|---|---|---|---|---|---|---|
| Eef1g | NP_080283.2 | Eef1g; 2610301D06Rik; Mm.247762; Mm.300099; EF1G; | 451 | 32% | 51% | 575 | 2e−48 |
| | | MGC103354 Protein with very strong similarity to eukaryotic | | | | | |
| | | elongation factor 1 gamma (human EEF1G), which is | | | | | |
| | | associated with gastric carcinoma, contains an elongation | | | | | |
| | | factor 1 gamma conserved domain and glutathione S- | | | | | |
| | | transferase N-terminal and C-terminal domains | | | | | |

Compared with C. elegans protein sequences (Documentation)

| | | | | | | | |
|---|---|---|---|---|---|---|---|
| F17C11.9 | CAA96631.1 | F17C11.9; F17C11.9A Protein involved in reproduction, | 421 | 31% | 47% | 490 | 8e−40 |
| | | embryogenesis, positive growth regulation, and | | | | | |
| | | regulation of DNA transposition | | | | | |

GTR2 - BAA28781.1
GTR2 blast vs human -

| | | | | | | | |
|---|---|---|---|---|---|---|---|
| RRAGC | NP_071440.1 | RRAGC; GTR2; RAGC; FLJ13311 Rag C protein, | 323 | 47% | 69% | 773 | 1e−79 |
| | | contains putative GTP-binding motifs, may play a role in | | | | | |
| | | tumor progression | | | | | |
| RRAGD | NP_067067.1 | RRAGD; RAGD; bA11D8.2.1; DKFZP761H171; RagD | 323 | 46% | 68% | 759 | 5e−78 |
| | | Rag D protein, member of the Rag A subfamily of the Ras- | | | | | |
| | | like small G protein superfamily, associates with the Ras- | | | | | |
| | | related GTP-binding protein Rag A (human RAGA) | | | | | |
| RRAGA | NP_006561.1 | RRAGA; RAGA; FIP-1; RagA Ras-related GTP binding | 237 | 30% | 49% | 212 | 4e−16 |
| | | protein, a GTP-binding protein that lacks GTPase activity, | | | | | |
| | | interacts with RAGC (GTR2), RAGD, and the adenovirus | | | | | |
| | | 14.7 kDa E3 protein, may be part of the tumor necrosis | | | | | |
| | | factor alpha (TNF) signaling pathway | | | | | |
| RRAGB | NP_057740.2 | RRAGB; RAGB; RagBs; RagB1; bA465E19.1 GTP- | 249 | 29% | 47% | 190 | 6e−12 |
| | | binding protein ragB, a Ras-related GTP-binding protein | | | | | |
| | | that may function in phosphate transport, signal | | | | | |
| | | transduction or cell proliferation | | | | | |

Compared with M. musculus protein sequences (Documentation)

| | | | | | | | |
|---|---|---|---|---|---|---|---|
| Rragc | NP_059503.1 | Rragc; (Gtr2); RAGC; TIB929; YGR163W; | 324 | 47% | 68% | 759 | 4e−78 |
| | | MGC47404; Mm.28746 Protein with high similarity | | | | | |
| | | to S. cerevisiae Gtr2p, which is a putative small | | | | | |
| | | GTPase, member of the Gtr1 or RagA G protein | | | | | |
| | | conserved region containing family | | | | | |
| Rragd | NP_081767.1 | Rragd; D4Ertd174e; 5730543C08Rik; | 318 | 46% | 68% | 736 | 3e−75 |
| | | C030003H22Rik Protein with high similarity to S. cerevisiae | | | | | |
| | | Gtr2p, which is a putative small GTPase, | | | | | |
| | | member of the Gtr1 or RagA G protein conserved | | | | | |
| | | region containing family | | | | | |
| Rraga | NP_848463.1 | Rraga; RAGA; FIP-1; 1300010C19Rik; Raga | 237 | 30% | 49% | 212 | 2e−14 |
| | | Protein with very strong similarity to ras-related | | | | | |
| | | GTP binding protein (rat Rraga), which is a GTP- | | | | | |
| | | binding protein that lacks GTPase activity and may | | | | | |
| | | function in signal transduction, member of the Gtr1 | | | | | |
| | | or RagA G protein conserved region containing | | | | | |
| | | family | | | | | |
| MGC69750 | NP_001004154.1 | MGC69750; Mm.190922; LOC245670; MGC95567 | 243 | 28% | 47% | 187 | 1e−11 |
| | | Protein with very strong similarity to GTP-binding | | | | | |
| | | protein ragB (rat RragB), which is a Ras-related | | | | | |
| | | GTPase and guanyl-nucleotide exchange factor that | | | | | |
| | | may act in cell proliferation, member of the Gtr1 or | | | | | |
| | | RagA G protein conserved region containing family | | | | | |

Compared with C. elegans protein sequences (Documentation)

| | | | | | | | |
|---|---|---|---|---|---|---|---|
| Y24F12A.2 | CAB60328.1 | Y24F12A.2 Protein involved in embryogenesis | 320 | 38% | 62% | 572 | 3e−59 |
| T24F1.1 | CAA90136.1 | T24F1.1 Protein with high similarity to ras-related GTP | 215 | 27% | 48% | 164 | 6e−11 |
| | | binding protein (rat Rraga), which is a GTP-binding | | | | | |
| | | protein that lacks GTPase activity and may play a role in | | | | | |
| | | signal transduction, member of the Gtr1 or RagA G | | | | | |
| | | protein conserved region containing family | | | | | |
| F57C12.2 | AAA83296.2 | F57C12.2 Protein that functions in genome stability of | 216 | 21% | 45% | 100 | 5e−04 |
| | | somatic cells | | | | | |

YGR054W - CAA97054.1
YGR054W blast vs human -

| | | | | | | | |
|---|---|---|---|---|---|---|---|
| eIF2A | NP_114414.2 | eIF2A; CDA02; eIF2a; MSTP004; MST089; MSTP089 | 651 | 27% | 44% | 548 | 2e−41 |
| | | Eukaryotic translation initiation factor 2A, a putative | | | | | |
| | | translation initiation factor | | | | | |
| EIF3S9 | NP_003742.2 | EIF3S9; EIF3-P116; EIF3-ETA; PRT1; (EIF3-P110) | 403 | 21% | 40% | 190 | 3e−13 |
| | | Eukaryotic translation initiation factor 3 subunit 9 eta 116 kDa, | | | | | |
| | | a subunit of the EIF3 complex that plays a role in | | | | | |
| | | protein synthesis initiation | | | | | |

Compared with M. musculus protein sequences (Documentation)

| | | | | | | | |
|---|---|---|---|---|---|---|---|
| D3Ertd194e | NP_001005509.1 | D3Ertd194e; D030048D22; MGC105246 Protein | 639 | 28% | 45% | 541 | 1e−40 |
| | | with low similarity to S. cerevisiae Ygr054p, which | | | | | |
| | | is a protein likely involved in translation initiation | | | | | |
| | | and may play role in signal transduction | | | | | |
| Eif3s9 | NP_598677.1 | Eif3s9; PRT1; eIF3s9; D5Wsu45e; EIF3-ETA; | 403 | 21% | 40% | 190 | 3e−13 |
| | | EIF3-P110; EIF3-P116 D5Wsu45e (eukaryotic | | | | | |
| | | translation initiation factor 3 subunit p116), a | | | | | |
| | | putative housekeeping protein and subunit of the | | | | | |
| | | eukaryotic translation initiation factor 3 complex, | | | | | |
| | | required for early stages of development | | | | | |

Compared with C. elegans protein sequences (Documentation)

| | | | | | | | |
|---|---|---|---|---|---|---|---|
| E04D5.1 | CAA91279.2 | E04D5.1; E04D5.1A Protein with low similarity to S. cerevisiae | 649 | 28% | 46% | 586 | 2e−50 |
| | | Ygr054p, which is a protein likely to be | | | | | |
| | | involved in translation initiation and may function in signal | | | | | |
| | | transduction | | | | | |

DAL80 - CAA82107.1
DAL80 blast vs human -

| | | | | | | | |
|---|---|---|---|---|---|---|---|
| GATA6 | NP_005248.2 | GATA6 GATA-binding protein 6, a member of GATA | 97 | 38% | 56% | 188 | 8e−15 |
| | | family of zinc-finger transcription factors, involved in | | | | | |
| | | the differentiation of vascular smooth muscle cells, | | | | | |
| | | implicated in cell proliferation and development; may be | | | | | |
| | | linked to sex cord-derived ovarian tumors | | | | | |
| GATA4 | NP_002043.2 | GATA4 GATA binding protein 4, a putative zinc-finger | 138 | 32% | 49% | 181 | 5e−14 |
| | | transcription factor that may play a role in heart and gut | | | | | |
| | | development, overproduction is detected in esophageal | | | | | |
| | | adenocarcinomas and adrenocortical carcinomas | | | | | |
| GATA5 | NP_536721.1 | GATA5; bB379O24.1 GATA binding protein 5, a | 51 | 55% | 76% | 177 | 1e−11 |
| | | putative transcriptional activator that may function in | | | | | |
| | | heart development | | | | | |
| TRPS1 | NP_054831.1 | TRPS1; GC79 Trichorhinophalangeal syndrome I, | 59 | 47% | 64% | 156 | 1e−10 |
| | | contains a GATA-type zinc finger and an Ikaros family | | | | | |
| | | domain, may act as a transcriptional repressor; mutation | | | | | |
| | | of the corresponding gene is associated with | | | | | |
| | | trichorhinophalangeal syndrome types I, II, and III | | | | | |
| GATA2 | NP_116027.2 | GATA2; NFE1B; MGC2306 GATA binding protein 2, | 133 | 34% | 47% | 179 | 3e−10 |
| | | transcriptional activator, regulates expression of | | | | | |
| | | erythroid-specific genes (perhaps in conjunction with | | | | | |
| | | GATA1), abnormal expression may play a role in | | | | | |
| | | leukemia; rat Gata2 is downregulated in Pneumocystis | | | | | |
| | | carinii infections | | | | | |
| GATA3 | NP_001002295.1 | GATA3; HDR; MGC5445; MGC2346; MGC5199 | 52 | 52% | 73% | 159 | 1e−09 |
| | | GATA-binding protein 3, a zinc-finger transcription | | | | | |
| | | factor, involved in T-cell differentiation, defense | | | | | |
| | | response, and embryogenesis, may be involved in some | | | | | |
| | | breast cancers, HDR syndrome, and transcriptional | | | | | |
| | | activation of HIV-1 | | | | | |
| GATA1 | NP_002040.1 | GATA1; ERYF1; (GF1); NFE1; GF-1; (NF-E1) GATA- | 51 | 53% | 71% | 156 | 2e−09 |
| | | binding protein 1, member of the GATA family of | | | | | |
| | | transcription factors that participates in erythropoiesis | | | | | |
| | | and is associated with Down syndrome-associated acute | | | | | |
| | | megakaryoblastic leukemia and transient | | | | | |
| | | myeloproliferative disorder upon gene mutation | | | | | |

Compared with M. musculus protein sequences (Documentation)

| | | | | | | | |
|---|---|---|---|---|---|---|---|
| Gata6 | NP_034388.2 | Gata6; Mm.33783; GATA-6 GATA-binding protein 6, a | 101 | 37% | 56% | 183 | 3e−14 |
| | | member of GATA family of Zinc-finger transcription factors | | | | | |
| | | that may play a critical role during differentiation and | | | | | |
| | | development and cell cycle arrest; human GATA6 may be | | | | | |
| | | linked to sex cord derived ovarian tumors | | | | | |
| Gata4 | NP_032118.2 | Gata4; Mm.1428; Gata-4 GATA-binding protein 4, zinc- | 63 | 49% | 68% | 179 | 5e−14 |
| | | finger transcription factor that plays a role in sex | | | | | |
| | | differentiation and heart and gut development; overproduction | | | | | |
| | | of human GATA4 is detected in esophageal adenocarcinomas | | | | | |
| | | and adrenocortical carcinomas | | | | | |
| Gata5 | NP_032119.1 | Gata5; Mm.2527; GATA-5 GATA binding protein 5, a | 139 | 31% | 50% | 188 | 8e−12 |
| | | transcriptional activator involved in endothelial cell | | | | | |
| | | differentiation, embryonic urogenital system development and | | | | | |
| | | possibly heart and lung development, may function in | | | | | |
| | | development of smooth muscle cell diversity | | | | | |
| Gata2 | NP_032116.3 | Gata2; Mm.1391; Gata-2 GATA binding protein 2, | 133 | 33% | 47% | 173 | 3e−10 |
| | | transcriptional activator, acts in hematopoiesis and urogenital | | | | | |
| | | development, potentiates generation of V2 interneurons; | | | | | |
| | | human GATA2 mis-expression may play a role in leukemia, | | | | | |
| | | rat Gata2 is downregulated in P. carinii infections | | | | | |
| Trps1 | NP_114389.1 | Trps1; D15Ertd586e; MGC46754 Trichorhinophalangeal | 59 | 47% | 64% | 156 | 1e−09 |
| | | syndrome I (human), has a GATA-type zinc finger and an | | | | | |
| | | Ikaros family domain, represses transcription mediated by | | | | | |
| | | other GATA factors; mutation of the human TRPS1 gene is | | | | | |
| | | linked to trichorhinophalangeal syndrome types I, II, and III | | | | | |
| Gata3 | NP_032117.1 | Gata3; Mm.606; Gata-3 GATA-binding protein 3, zinc-finger | 52 | 52% | 73% | 159 | 5e−09 |
| | | transcription factor, involved in T-cell differentiation, defense | | | | | |
| | | response, neurogenesis, and cell proliferation, may be | | | | | |
| | | involved in asthma; human GATA3 may be involved in breast | | | | | |
| | | cancer, HDR syndrome, HIV-1 activation | | | | | |
| Gata1 | NP_032115.1 | Gata1; Gata-1; Gf-1 GATA-binding protein 1, member of the | 63 | 44% | 60% | 158 | 7e−09 |
| | | GATA family of transcription factors that acts in | | | | | |
| | | erythropoiesis and regulates Sertoli cell gene expression; | | | | | |
| | | human GATA1 is is associated with acute megakaryoblastic | | | | | |
| | | leukemia and transient myeloproliferative disorder | | | | | |

Compared with C. elegans protein sequences (Documentation)

| | | | | | | | |
|---|---|---|---|---|---|---|---|
| Y48A5B.C | | Y48A5B.C Protein with strong similarity to C. elegans | 86 | 41% | 62% | 177 | 1e−11 |
| | | ELT-6, which is required for embryonic development | | | | | |
| | | and functions to repress vulval cell fusion and regulate | | | | | |
| | | cell fate determination, contains a GATA-type zinc | | | | | |
| | | finger domain | | | | | |
| elt-6 | AAC68957.3 | elt-6; F52C12.5 Erythroid-like-transcription factor 6, | 86 | 41% | 62% | 177 | 1e−11 |
| | | protein required for embryonic development, functions to | | | | | |
| | | repress vulval cell fusion and regulate cell fate | | | | | |
| | | determination | | | | | |
| elt-1 | CAA92494.1 | elt-1; W09C2.1 Erythroid-like transcription factor 1, | 51 | 57% | 71% | 158 | 1e−08 |
| | | GATA transcription factor involved in embryogenesis, | | | | | |
| | | regulation of movement, egg-laying, and adult life span | | | | | |
| | | determination, activates LIN-26 in the hypodermis, may | | | | | |
| | | act to specify the major hypodermal cell fate | | | | | |
| elt-2 | CAA90029.2 | elt-2; C33D3.1 Erythroid-like transcription factor 2, | 68 | 37% | 57% | 145 | 1e−08 |
| | | GATA-type zinc finger DNA-binding factor involved in | | | | | |
| | | development of the gut, larval growth, reproduction, | | | | | |
| | | regulation of movement, and osmoregulation | | | | | |
| egl-18 | AAD36952.2 | egl-18; elt-5; F55A8.1 Egg-laying abnormal 18, protein | 118 | 31% | 47% | 150 | 5e−08 |
| | | required for embryonic development, functions to repress | | | | | |
| | | vulval cell fusion and regulate cell fate determination | | | | | |
| elt-4 | CAD44111.1 | elt-4; C39B10.6 Erythroid-like transcription factor 4, | 41 | 54% | 61% | 125 | 2e−07 |
| | | small GATA-type zinc finger domain-containing protein | | | | | |
| | | that binds DNA weakly and non-specifically | | | | | |
| elt-3 | | elt-3; K02B9.4 Erythroid-like transcription factor family | 86 | 40% | 52% | 157 | 3e−07 |
| | | 3, GATA-binding factor that is required for hypodermal | | | | | |
| | | cell differentiation | | | | | |
| C18G1.2 | AAC17756.1 | C18G1.2 Protein containing a GATA-type zinc finger | 55 | 42% | 58% | 115 | 5e−05 |
| | | domain, has low similarity to a region of C. elegans | | | | | |
| | | ELT-3, which is a GATA-binding factor required for | | | | | |
| | | hypodermal cell differentiation | | | | | |
| end-1 | CAB04513.1 | end-1; F58E10.2 Endoderm specification 1, GATA | 49 | 45% | 59% | 120 | 8e−05 |
| | | transcription factor expressed in zygotes and required for | | | | | |
| | | development of the gut | | | | | |
| end-3 | CAB04516.1 | end-3; F58E10.5 Endoderm determining 3, protein | 52 | 38% | 52% | 107 | 1e−04 |
| | | involved in differentiation of intestinal cells, | | | | | |
| | | transcriptional target of the repressor POP-1 and the | | | | | |
| | | activator MED-1 in the EMS lineage | | | | | |

AGP1 - CAA42360.2
AGP1 blast vs human -

| | | | | | | | |
|---|---|---|---|---|---|---|---|
| SLC7A5 | NP_003477.3 | SLC7A5; D16S469E; MPE16; E16; LAT1; (CD98); | 435 | 23% | 42% | 181 | 3e−07 |
| | | hLat; LOC51597 Solute carrier family 7 member 5, | | | | | |
| | | an L-type and neutral amino acid transporter, binds | | | | | |
| | | CD98 heavy chain (SLC3A2) to mediate large | | | | | |
| | | neutral amino acid transport, increased expression | | | | | |
| | | may correlate with disease progression in colon | | | | | |
| | | cancer | | | | | |
| KIAA1613 | NP_066000.1 | KIAA1613 Member of the amino acid permease | 454 | 21% | 40% | 197 | 1e−06 |
| | | family of membrane transporters, has moderate | | | | | |
| | | similarity to solute carrier family 7 (cationic amino | | | | | |
| | | acid transporter) member 1 (rat Slc7a1), which | | | | | |
| | | mediates the transport of basic amino acids | | | | | |
| SLC7A8 | NP_036376.2 | SLC7A8; LAT2; LPI-PC1 Solute carrier family 7 | 364 | 23% | 40% | 177 | 1e−05 |
| | | member 8 (L amino acid transporter 2), a sodium | | | | | |
| | | independent neutral amino acid transporter that | | | | | |
| | | induces system L transport activity as a complex with | | | | | |
| | | 4F2hc (SLC3A2), may be involved in epithelial | | | | | |
| | | amino acid absorption | | | | | |
| SLC7A11 | NP_055146.1 | SLC7A11; XCT; xCT; CCBR1 Solute carrier family | 371 | 21% | 39% | 135 | 7e−05 |
| | | 7 member 11 (cationic amino acid transporter y+ | | | | | |
| | | system), a cystine and glutamate transporter, activity | | | | | |
| | | requires the heavy chain of 4F2 cell surface antigen | | | | | |
| | | (SLC3A2), may act in oxidative stress response and | | | | | |
| | | cisplatin resistance | | | | | |
| SLC7A2 | NP_001008539.1 | SLC7A2; ATRC2; HCAT2; (CAT-2) Solute carrier | 416 | 22% | 39% | 127 | 2e−04 |
| | | family 7 member 2 (cationic amino acid transporter 2 | | | | | |
| | | y+ system), functions in the transport of basic amino | | | | | |
| | | acids | | | | | |
| SLC7A10 | NP_062823.1 | SLC7A10; ASC-1; FLJ20839; HASC-1; asc-1 Solute | 416 | 21% | 39% | 142 | 3e−04 |
| | | carrier family 7 member 10 (cationic amino acid | | | | | |
| | | transporter y+ system), mediates sodium-independent | | | | | |
| | | transport of neutral amino acids, requires SLC3A2 | | | | | |
| | | for proper function, may mobilize D-serine in the | | | | | |
| | | brain; mutations may result in cystinuria | | | | | |
| SLC7A1 | NP_003036.1 | SLC7A1; ATRC1; CAT-1; ERR; REC1L; HCAT1 | 398 | 25% | 41% | 170 | 7e−04 |
| | | Solute carrier family 7 (cationic amino acid | | | | | |
| | | transporter) member 1, a cationic amino acid | | | | | |
| | | transporter that is a y(+) system transporter, | | | | | |
| | | transports basic amino acids such as arginine and | | | | | |
| | | lysine, part of the y(+) transport system | | | | | |

Compared with M. musculus protein sequences (Documentation)

| | | | | | | | |
|---|---|---|---|---|---|---|---|
| Slc7a5 | NP_035534.2 | Slc7a5; Mm.27943; TA1; D0H16S474E Solute carrier | 444 | 24% | 43% | 196 | 5e−07 |
| | | family 7 member 5, an L-type and neutral amino acid | | | | | |
| | | transporter, also transports L DOPA, binds CD98 heavy | | | | | |
| | | chain (Slc3a2) to mediate amino acid transport; increased | | | | | |
| | | human SLC7A5 levels may correlate with disease | | | | | |
| | | progression in colon cancer | | | | | |
| Slc7a11 | NP_036120.1 | Slc7a11; Xct; Mm.42036; xCT Solute carrier family 7 | 422 | 23% | 39% | 156 | 3e−06 |
| | | member 11 (cationic amino acid transporter y+ system), a | | | | | |
| | | cystine and glutamate transporter, activity requires | | | | | |
| | | interaction with Slc3a1 or Slc3a2, may serve in the | | | | | |
| | | oxidative stress response and redox homeostasis | | | | | |
| Slc7a8 | NP_058668.1 | Slc7a8; LAT2 Solute carrier family 7 member 8 (L amino | 384 | 24% | 41% | 190 | 1e−05 |
| | | acid transporter 2), a sodium independent neutral amino | | | | | |
| | | acid transporter that induces system L transport activity | | | | | |
| | | as a complex with 4F2hc (Slc3a2), may be involved in | | | | | |
| | | epithelial amino acid absorption | | | | | |
| Slc7a10 | NP_059090.2 | Slc7a10; Asc-1; D7Bwg0847e Solute carrier family 7 | 416 | 22% | 39% | 147 | 5e−05 |
| | | member 10 (cationic amino acid transporter y+ system), | | | | | |
| | | mediates sodium- and chloride-independent transport of | | | | | |
| | | small neutral amino acids and alpha aminoisobutyric | | | | | |
| | | acid; mutation of human SLC7A10 may result in | | | | | |
| | | cystinuria | | | | | |
| Slc7a1 | NP_031539.1 | Slc7a1; Atrc-1; Rec-1; Rev-1; Atrc1; Mm.5255; CAT-1; | 415 | 23% | 40% | 150 | 1e−04 |
| | | 4831426K01Rik; mCAT-1 Solute carrier family 7 | | | | | |
| | | (cationic amino acid transporter) member 1, a cationic | | | | | |
| | | amino acid transporter that is a y(+) system transporter, | | | | | |
| | | acts as an ecotropic murine leukemia retrovirus receptor, | | | | | |
| | | required for developmental hematopoiesis and growth | | | | | |
| | | control | | | | | |
| Slc7a9 | NP_067266.1 | Slc7a9; CSNU3 Solute carrier family 7 member 9 | 404 | 22% | 39% | 127 | 4e−04 |
| | | (cationic amino acid transporter, y+ system), mediates the | | | | | |
| | | transport of cystine and dibasic amino acids; mutations in | | | | | |
| | | the human SLC7A9 gene are associated with non-type I | | | | | |
| | | cystinuria | | | | | |
| Slc7a12 | NP_543128.1 | Slc7a12; Asc-2; XAT1 Solute carrier family 7 (cationic | 359 | 19% | 41% | 110 | 5e−04 |
| | | amino acid transporter, y+ system) member 12, mediates | | | | | |
| | | transport of primarily small, neutral amino acids, | | | | | |
| | | expression in reticulocytes of the spleen is induced by | | | | | |
| | | experimental hemolytic anemia | | | | | |
| BC061928 | NP_766449.1 | BC061928; A930013N06 Member of the amino acid | 454 | 21% | 39% | 189 | 8e−04 |
| | | permease family of membrane transporters, has moderate | | | | | |
| | | similarity to solute carrier family 7 member 1 (human | | | | | |
| | | SLC7A1), which is a cationic amino acid transporter that | | | | | |
| | | is a y(+) system transporter and transports basic amino | | | | | |
| | | acids | | | | | |

Compared with C. elegans protein sequences (Documentation)

| | | | | | | | |
|---|---|---|---|---|---|---|---|
| Aat-1 | CAA92459.1 | aat-1; F27C8.1 Amino acid transporter catalytic chain 1, acts | 467 | 21% | 39% | 170 | 6e−05 |
| | | as an obligatory amino acid exchanger when complexed with | | | | | |
| | | ATG-2/C38C6.2, transporting small neutral amino acids and | | | | | |
| | | some larger aromatic amino acids, involved in the efflux of L- | | | | | |
| | | alanine | | | | | |

GTR1 - CAA89159.1
GTR1 blast vs human -

| | | | | | | | |
|---|---|---|---|---|---|---|---|
| RRAGB | NP_057740.2 | RRAGB; RAGB; RagBs; RagB1; bA465E19.1 GTP- | 336 | 48% | 63% | 743 | 8e−73 |
| | | binding protein ragB, a Ras-related GTP-binding protein | | | | | |
| | | that may function in phosphate transport, signal | | | | | |
| | | transduction or cell proliferation | | | | | |
| RRAGA | NP_006561.1 | RRAGA; RAGA; FIP-1; RagA Ras-related GTP binding | 308 | 52% | 69% | 778 | 8e−67 |
| | | protein, a GTP-binding protein that lacks GTPase activity, | | | | | |
| | | interacts with RAGC (GTR2), RAGD, and the adenovirus | | | | | |
| | | 14.7 kDa E3 protein, may be part of the tumor necrosis | | | | | |
| | | factor alpha (TNF) signaling pathway | | | | | |
| RRAGC | NP_071440.1 | RRAGC; GTR2; RAGC; FLJ13311 Rag C protein, | 308 | 22% | 48% | 191 | 4e−07 |
| | | contains putative GTP-binding motifs, may play a role in | | | | | |
| | | tumor progression | | | | | |
| RRAGD | NP_067067.1 | RRAGD; RAGD; bA11D8.2.1; DKFZP761H171; RagD | 294 | 23% | 47% | 169 | 3e−05 |
| | | Rag D protein, member of the Rag A subfamily of the Ras- | | | | | |
| | | like small G protein superfamily, associates with the Ras- | | | | | |
| | | related GTP-binding protein Rag A (human RAGA) | | | | | |
| RAB7B | NP_796377.2 | RAB7B; (RAB7); MGC9726; MGC16212 RAB7B | 143 | 32% | 50% | 98 | 7e−04 |
| | | member RAS oncogene family, a lysosome-associated | | | | | |
| | | small GTPase that is involved in monocytic differentiation | | | | | |
| | | of acute promyelocytic leukemia cells | | | | | |

Compared with M. musculus protein sequences (Documentation)

| | | | | | | | |
|---|---|---|---|---|---|---|---|
| Rraga | NP_848463.1 | Rraga; RAGA; FIP-1; 1300010C19Rik; Raga | 308 | 52% | 69% | 778 | 2e−77 |
| | | Protein with very strong similarity to ras-related | | | | | |
| | | GTP binding protein (rat Rraga), which is a GTP- | | | | | |
| | | binding protein that lacks GTPase activity and may | | | | | |
| | | function in signal transduction, member of the Gtr1 | | | | | |
| | | or RagA G protein conserved region containing | | | | | |
| | | family | | | | | |
| MGC69750 | NP_001004154.1 | MGC69750; Mm.190922; LOC245670; MGC95567 | 336 | 47% | 63% | 739 | 1e−71 |
| | | Protein with very strong similarity to GTP-binding | | | | | |
| | | protein ragB (rat RragB), which is a Ras-related | | | | | |
| | | GTPase and guanyl-nucleotide exchange factor that | | | | | |
| | | may act in cell proliferation, member of the Gtr1 or | | | | | |
| | | RagA G protein conserved region containing family | | | | | |
| Rragc | NP_059503.1 | Rragc; (Gtr2); RAGC; TIB929; YGR163W; | 308 | 22% | 47% | 189 | 3e−07 |
| | | MGC47404; Mm.28746 Protein with high similarity | | | | | |
| | | to S. cerevisiae Gtr2p, which is a putative small | | | | | |
| | | GTPase, member of the Gtr1 or RagA G protein | | | | | |
| | | conserved region containing family | | | | | |
| Rragd | NP_081767.1 | Rragd; D4Ertd174e; 5730543C08Rik; | 288 | 23% | 46% | 154 | 1e−04 |
| | | C030003H22Rik Protein with high similarity to S. cerevisiae | | | | | |
| | | Gtr2p, which is a putative small GTPase, | | | | | |
| | | member of the Gtr1 or RagA G protein conserved | | | | | |
| | | region containing family | | | | | |

Compared with C. elegans protein sequences (Documentation)

| | | | | | | | |
|---|---|---|---|---|---|---|---|
| T24F1.1 | CAA90136.1 | T24F1.1 Protein with high similarity to ras-related GTP | 318 | 47% | 65% | 713 | 7e−60 |
| | | binding protein (rat Rraga), which is a GTP-binding | | | | | |
| | | protein that lacks GTPase activity and may play a role in | | | | | |
| | | signal transduction, member of the Gtr1 or RagA G | | | | | |
| | | protein conserved region containing family | | | | | |
| F57C12.2 | AAA83296.2 | F57C12.2 Protein that functions in genome stability of | 220 | 30% | 54% | 269 | 1e−12 |
| | | somatic cells | | | | | |
| Y24F12A.2 | CAB60328.1 | Y24F12A.2 Protein involved in embryogenesis | 221 | 21% | 46% | 123 | 7e−07 |
| F19H8.3 | CAB07583.1 | F19H8.3 Protein with high similarity to ADP- | 143 | 24% | 43% | 96 | 4e−04 |
| | | ribosylation factor like 3 (human ARL3), which binds | | | | | |
| | | GTP and may act in intracellular protein trafficking, | | | | | |
| | | member of an uncharacterized GTPase family, contains | | | | | |
| | | an ADP-ribosylation factor (ARF) family domain | | | | | |

RPS25A - CAA97010.1
RPS25A blast vs. human -

| | | | | | | | |
|---|---|---|---|---|---|---|---|
| RPS25 | NP_001019.1 | RPS25; Hs.512676; LOC6230 Ribosomal protein S25, a | 113 | 46% | 64% | 243 | 6e−15 |
| | | putative RNA-binding component of the small 40S | | | | | |
| | | ribosomal subunit that may play a role in protein | | | | | |
| | | biosynthesis | | | | | |

Compared with M. musculus protein sequences (Documentation)

| | | | | | | | |
|---|---|---|---|---|---|---|---|
| Rps25 | NP_077228.1 | Rps25; 2810009D21Rik Protein with high similarity to C. elegans | 113 | 46% | 64% | 243 | 3e−15 |
| | | K02B2.5, which is involved in positive growth | | | | | |
| | | regulation, member of the S25 ribosomal protein family | | | | | |

Compared with C. elegans protein sequences (Documentation)

| | | | | | | | |
|---|---|---|---|---|---|---|---|
| K02B2.5 | AAK39246.1 | K02B2.5 Protein involved in positive growth | 109 | 40% | 62% | 226 | 5e−13 |
| | | regulation | | | | | |

TOR1 - AAB39292.1
TOR1 blast vs. human -

| | | | | | | | |
|---|---|---|---|---|---|---|---|
| FRAP1 | NP_004949.1 | FRAP1; FRAP2; FRAP; MTOR; RAFT1; Hs.155952; | 2586 | 39% | 58% | 4525 | 0.0 |
| | | RAPT1 FK506 binding protein 12-rapamycin | | | | | |
| | | associated protein 1, a serine-threonine and 1- | | | | | |
| | | phosphatidylinositol 4-kinase that regulates translation, | | | | | |
| | | cell cycle, and p53 (TP53)-dependent apoptosis; | | | | | |
| | | inhibition may be therapeutic for various types of | | | | | |
| | | cancer | | | | | |
| SMG1 | NP_055907.2 | SMG1; (ATX); LIP; KIAA0421; Hs.352382; 61E3.4 | 643 | 27% | 45% | 595 | 5e−54 |
| | | PI-3-kinase-related kinase SMG-1, a protein kinase that | | | | | |
| | | participates in nonsense-mediated mRNA decay by | | | | | |
| | | phosphorylating hUpf1 (RENT1), binds to and activates | | | | | |
| | | atypical protein kinase C lambda (PRKCL) | | | | | |
| ATR | NP_001175.1 | ATR; (FRP1); SCKL; SCKL1 Ataxia telangiectasia and | 1168 | 25% | 42% | 639 | 1e−49 |
| | | Rad3 related, a PIK-related protein kinase that functions | | | | | |
| | | in DNA damage monitoring, checkpoint-mediated cell | | | | | |
| | | cycle control, and possibly recombination, | | | | | |
| | | overexpression may inhibit differentiation and induce | | | | | |
| | | aneuploidy | | | | | |
| ATM | NP_000042.2 | ATM; (AT1); ATA; ATC; (ATD); (ATDC); (TRIM29); | 1056 | 23% | 41% | 499 | 2e−46 |
| | | ATE Ataxia telangiectasia mutated, a serine/threonine | | | | | |
| | | kinase involved in apoptosis, DNA stability, cell cycle, | | | | | |
| | | and radiation response; gene mutation is associated with | | | | | |
| | | ataxia telangiectasia and implicated in B cell chronic | | | | | |
| | | lymphocytic leukemia | | | | | |
| PRKDC | NP_008835.5 | PRKDC; DNPK1; HYRC1; DNAPK; XRCC7; DNA- | 532 | 26% | 48% | 383 | 1e−34 |
| | | PKcs; p350; HYRC DNA-dependent protein kinase | | | | | |
| | | catalytic subunit, a DNA-binding protein kinase | | | | | |
| | | involved in DNA double-strand break repair, V(D)J | | | | | |
| | | recombination, and radiation response, phosphorylates | | | | | |
| | | and activates AKT; mouse Prkdc deficiency is | | | | | |
| | | associated with SCID | | | | | |
| TRRAP | NP_003487.1 | TRRAP; TR-AP; PAF400; Hs.203952 Transformation | 1352 | 21% | 38% | 259 | 1e−14 |
| | | transcription domain-associated protein, ATM | | | | | |
| | | superfamily member, subunit of histone acetylase, | | | | | |
| | | adenovirus E1A binding, and ESR1 coactivator | | | | | |
| | | complexes, transcription coactivator for MYC and E2F, | | | | | |
| | | may affect breast cancer cell proliferation | | | | | |
| PIK3CD | NP_005017.2 | PIK3CD; p110delta; Hs.166116; p110D | 339 | 26% | 42% | 181 | 4e−11 |
| | | Phosphatidylinositol 3′-kinase delta catalytic subunit, a | | | | | |
| | | kinase which forms a complex with the regulatory | | | | | |
| | | subunit p85alpha (PIK3R1) or p85beta (PIK3R2), | | | | | |
| | | involved in transmembrane signaling, may play a role in | | | | | |
| | | cytoskeletal functions | | | | | |
| PIK3C3 | NP_002638.2 | PIK3C3; VPS34; Vps34 Phosphatidylinositol 3-kinase | 304 | 26% | 42% | 166 | 9e−10 |
| | | class 3, phosphorylates PtdIns but not PtdIns4P or | | | | | |
| | | PtdIns(4,5)P2, induces macroautophagy, predicted to be | | | | | |
| | | involved in vesicular trafficking | | | | | |
| PIK3CB | NP_006210.1 | PIK3CB; PIK3C1; (PI3K); (p110); p110-BETA; | 266 | 27% | 43% | 165 | 1e−09 |
| | | PI3Kbeta Catalytic beta subunit of phosphatidylinositol | | | | | |
| | | 3-kinase, a class IA phosphoinositide 3-kinase subunit | | | | | |
| | | that forms heterodimers with various regulatory or | | | | | |
| | | adaptor subunits, involved in multiple signal | | | | | |
| | | transduction pathways during cell proliferation | | | | | |
| PIK3CA | NP_006209.2 | PIK3CA; p110alpha; (PI3K); p110-alpha | 230 | 26% | 44% | 143 | 2e−08 |
| | | Phosphatidylinositol 3-kinase catalytic alpha subunit, | | | | | |
| | | heterodimerizes with an 85-kDa regulatory subunit that | | | | | |
| | | binds the kinase to receptors for signal transduction, | | | | | |
| | | expression, activity and gene amplification are involved | | | | | |
| | | in cancer progression | | | | | |
| PIK3C2A | NP_002636.1 | PIK3C2A; PI3-K-C2(ALPHA); CPK; PI3-K-C2A; | 232 | 25% | 41% | 144 | 6e−08 |
| | | PI3K-C2alpha Phosphoinositide-3-kinase class 2 alpha | | | | | |
| | | polypeptide, phosphorylates only PtdIns and PtdIns4P | | | | | |
| | | in the absence of phosphatidylserine but phosphorylates | | | | | |
| | | PtdIns(4,5)P2 in the presence of phosphatidylserine, | | | | | |
| | | exhibits insensitivity to wortmannin | | | | | |
| PIK3CG | NP_002640.2 | PIK3CG; p110gamma; (PI3K); PIK3; PI3CG; | 346 | 24% | 42% | 144 | 7e−07 |
| | | PI3Kgamma Phosphoinositide-3-kinase catalytic | | | | | |
| | | gamma, a lipid kinase activated by G beta-gamma | | | | | |
| | | subunits and H-Ras (HRAS), mediates | | | | | |
| | | lysophosphatidylcholine signaling and actin | | | | | |
| | | cytoskeletal rearrangement; expression is lost in | | | | | |
| | | colorectal adenocarcinoma | | | | | |
| LOC220686 | NP_954977.2 | LOC220686 Member of the phosphatidylinositol 3- and | 196 | 23% | 41% | 112 | 9e−06 |
| | | 4-kinase family, has high similarity to a region of | | | | | |
| | | human PIK4CA, which is a type II phosphatidylinositol | | | | | |
| | | 4-kinase that catalyzes the first step in | | | | | |
| | | phosphatidylinositol 4,5-bisphosphate biosynthesis | | | | | |
| PIK3C2B | NP_002637.2 | PIK3C2B; C2-PI3K; PI3K-C2beta; Hs.132463 | 296 | 22% | 41% | 126 | 5e−05 |
| | | Phosphoinositide-3-kinase class 2 beta polypeptide, a | | | | | |
| | | nuclear enzyme that catalyzes phosphorylation of | | | | | |
| | | phosphatidylinositol and phosphatidylinositol 4 | | | | | |
| | | monophosphate, may act in signal transduction | | | | | |
| LOC375133 | NP_955377.2 | LOC375133 Member of the phosphatidylinositol 3- and | 46 | 46% | 70% | 105 | 5e−05 |
| | | 4-kinase family, has high similarity to a region of | | | | | |
| | | phosphatidylinositol 4-kinase catalytic alpha | | | | | |
| | | polypeptide (human PIK4CA), which is a type II | | | | | |
| | | phosphatidylinositol 4-kinase that is inhibited by | | | | | |
| | | adenosine | | | | | |
| PIK4CA | NP_477352.1 | PIK4CA; PI4K-ALPHA; pi4K230 Phosphatidylinositol | 196 | 23% | 41% | 113 | 2e−04 |
| | | 4-kinase catalytic alpha polypeptide, a type II | | | | | |
| | | phosphatidylinositol 4-kinase that catalyzes the first | | | | | |
| | | step in phosphatidylinositol 4,5-bisphosphate | | | | | |
| | | biosynthesis; activity is enhanced by detergent and | | | | | |
| | | inhibited by adenosine | | | | | |
| FLJ12688 | BAB21837.1 | FLJ12688; KIAA1746 Protein containing three HEAT | 163 | 24% | 44% | 101 | 4e−04 |
| | | repeats, which appear to act as protein binding surfaces, | | | | | |
| | | has a region of weak similarity to a region of C. elegans | | | | | |
| | | T16G12.5, which is involved in epithelium | | | | | |
| | | morphogenesis and regulation of movement and vulva | | | | | |
| | | development | | | | | |

Compared with M. musculus protein sequences (Documentation)

| | | | | | | | |
|---|---|---|---|---|---|---|---|
| Frap1 | NP_064393.1 | Frap1; 2610315D21Rik; FRAP; mTOR; MTOR; | 2589 | 39% | 58% | 4510 | 0.0 |
| | | FRAP2; RAFT1; RAPT1; flat FK506 binding | | | | | |
| | | protein 12-rapamycin associated protein 1, a | | | | | |
| | | serine-threonine kinase that regulates translation, | | | | | |
| | | cell cycle, and development, involved in | | | | | |
| | | starvation responses; inhibition of human FRAP1 | | | | | |
| | | may be therapeutic for various types of cancer | | | | | |
| Atm | NP_031525.1 | Atm; Mm.5088 Ataxia telangiectasia mutated, a | 456 | 30% | 49% | 476 | 2e−44 |
| | | serine/threonine kinase involved in apoptosis, | | | | | |
| | | DNA stability, cell cycle and radiation response; | | | | | |
| | | human ATM mutation is associated with ataxia | | | | | |
| | | telangiectasia and implicated in B cell chronic | | | | | |
| | | lymphocytic leukemia | | | | | |
| Prkdc | NP_035289.1 | Prkdc; Mm.71; p460; DNAPK; slip; DNAPDcs; | 555 | 25% | 45% | 373 | 6e−33 |
| | | DNA-PKcs; scid; DNPK1; HYRC1; XRCC7; | | | | | |
| | | DNA-PK DNA-dependent protein kinase | | | | | |
| | | catalytic subunit, a DNA-binding protein kinase | | | | | |
| | | involved in DNA double-strand break repair and | | | | | |
| | | V(D)J recombination; absence is associated with | | | | | |
| | | severe combined immunodeficiency | | | | | |
| Atr | AAF61728.1 | Atr Ataxia telangiectasia and Rad3 related, a | 304 | 31% | 47% | 329 | 1e−27 |
| | | PIK-related protein kinase required for genomic | | | | | |
| | | integrity and early embryonic development, may | | | | | |
| | | function in DNA repair or recombination during | | | | | |
| | | meiosis, regulates the checkpoint response to | | | | | |
| | | ionizing radiation | | | | | |
| Pik3cd | NP_032866.1 | Pik3cd; p100_delta; p110delta; 2410099E07Rik; | 285 | 27% | 44% | 180 | 2e−11 |
| | | signalling Phosphatidylinositol 3-kinase catalytic | | | | | |
| | | delta polypeptide, a putative lipid kinase | | | | | |
| | | expressed in spleen and testis, may play a role in | | | | | |
| | | signaling in the immune system; mutation in the | | | | | |
| | | corresponding gene causes inflammatory bowel | | | | | |
| | | disease | | | | | |
| Pik3c3 | NP_852079.2 | Pik3c3; Vps34; 5330434F23; 5330434F23Rik; | 304 | 27% | 42% | 172 | 6e−11 |
| | | Mm.194127 Protein with very strong similarity | | | | | |
| | | to rat Pik3c3, member of the phosphoinositide 3- | | | | | |
| | | kinase family accessory domain containing | | | | | |
| | | family and the phosphatidylinositol 3- and 4- | | | | | |
| | | kinase family, contains a phosphoinositide 3- | | | | | |
| | | kinase C2 domain | | | | | |
| Pik3cb | NP_083370.1 | Pik3cb; 1110001J02Rik; p110beta Catalytic beta | 266 | 28% | 42% | 162 | 6e−08 |
| | | subunit of phosphatidylinositol 3-kinase, a class | | | | | |
| | | IA phosphoinositide 3-kinase subunit that forms | | | | | |
| | | heterodimers with various regulatory subunits, | | | | | |
| | | involved in multiple signal transduction | | | | | |
| | | pathways, required for embryonic development. | | | | | |
| Pik3ca | NP_032865.1 | Pik3ca; Mm.41943; p110; caPI3K; (PI3K); | 230 | 26% | 44% | 143 | 1e−07 |
| | | 6330412C24Rik Phosphatidylinositol 3-kinase | | | | | |
| | | catalytic alpha subunit, heterodimerizes with an | | | | | |
| | | 85-kDa regulatory subunit that binds the kinase | | | | | |
| | | to receptors for signal transduction; human | | | | | |
| | | PIK3CA expression, activity, gene amplification | | | | | |
| | | are involved in cancer progression | | | | | |
| Pik3c2a | NP_035213.1 | Pik3c2a; Mm.3810; Cpk-m Phosphoinositide-3- | 232 | 25% | 41% | 141 | 1e−07 |
| | | kinase C2 domain-containing alpha polypeptide, | | | | | |
| | | phosphorylates PtdIns and PtdIns-4-P but not | | | | | |
| | | PtdIns(4,5)P2, exhibits some insensitivity to | | | | | |
| | | wortmannin, contains a C-terminal C2 domain | | | | | |
| Pik3cg | NP_064668.1 | Pik3cg; PI3Kgamma; p110gamma; | 347 | 23% | 41% | 143 | 5e−07 |
| | | 5830428L06Rik Phosphoinositide-3-kinase | | | | | |
| | | catalytic gamma, a lipid kinase catalyzing | | | | | |
| | | Ptdins(3,4,5)P3 formation, involved in mast cell | | | | | |
| | | degranulation, neutrophil chemotaxis and | | | | | |
| | | activation, and T-cell development; human | | | | | |
| | | PIK3CG expression is lost in colorectal | | | | | |
| | | adenocarcinoma | | | | | |
| 2610207I05Rik | BAC97946.1 | 2610207I05Rik; mKIAA0421 Member of the | 55 | 38% | 64% | 109 | 1e−05 |
| | | FRAP, ATM, TRRAP C-terminal (FATC) | | | | | |
| | | domain family, has very strong similarity to a | | | | | |
| | | region of PI-3-kinase-related kinase SMG-1 | | | | | |
| | | (human SMG1), which is a protein kinase that | | | | | |
| | | acts in nonsense-mediated mRNA decay by | | | | | |
| | | phosphorylating human RENT1 | | | | | |
| Pik4ca | NP_001001983.1 | Pik4ca; LOC224020 Protein with very strong | 192 | 24% | 41% | 112 | 4e−05 |
| | | similarity to rat Pik4ca, which is a | | | | | |
| | | phosphatidylinositol (PI) 4-kinase that catalyzes | | | | | |
| | | PI 4,5-bisphosphate biosynthesis, member of the | | | | | |
| | | phosphoinositide 3-kinase family accessory | | | | | |
| | | domain containing and PI 3- and 4-kinase | | | | | |
| | | families | | | | | |

Compared with C. elegans protein sequences (Documentation)

| | | | | | | | |
|---|---|---|---|---|---|---|---|
| let-363 | AAN84885.1 | let-363; B0261.2A; Ce-tor; B0261.2 Lethal 363, target- | 2720 | 30% | 49% | 2952 | e−162 |
| | | of-rapamycin-like protein kinase involved in larval | | | | | |
| | | development of the gut and gonad, metabolism, and life | | | | | |
| | | span regulation, functions with DAF-15 and interacts | | | | | |
| | | with the insulin-signaling pathway during dauer | | | | | |
| | | formation | | | | | |
| smg-1 | AAC48167.3 | smg-1; C48B6.6A; mab-1; C48B6.6 Suppressor with | 633 | 27% | 44% | 532 | 4e−47 |
| | | morphological effect on genitalia 1, PI-3-related protein | | | | | |
| | | kinase required for nonsense-mediated mRNA decay, | | | | | |
| | | mRNA surveillance, functions in the phosphorylation of | | | | | |
| | | SMG-2 | | | | | |
| atm-1 | AAF60692.2 | atm-1; Y48G1BL.F; Y48G1BL.2 May function in a | 440 | 23% | 42% | 297 | 4e−24 |
| | | DNA damage checkpoint pathway, has strong similarity | | | | | |
| | | to S. cerevisiae TEL1 which is a phosphatidylinositol 3- | | | | | |
| | | kinase (PI kinase) homolog involved in controlling | | | | | |
| | | telomere length | | | | | |
| atl-1 | CAA94790.2 | atl-1; T06E4.3A; Ce-atl1; T06E4.3 ATM-like 1, | 409 | 25% | 42% | 278 | 1e−21 |
| | | putative PI-3-like kinase that is required for an early | | | | | |
| | | embryonic GOA-1-, GPA-16-dependent DNA | | | | | |
| | | replication checkpoint involved in chromosome | | | | | |
| | | stability, functions in cell division asynchrony in two- | | | | | |
| | | cell embryos by delaying mitotic entry by P1 | | | | | |
| vps-34 | AAF23184.1 | vps-34; B0025.1A; let-512; vps34; B0025.1 Related to | 547 | 24% | 39% | 169 | 9e−10 |
| | | yeast vacuolar protein sorting factor 34, | | | | | |
| | | phosphatidylinositol 3-kinase required for receptor- | | | | | |
| | | mediated endocytosis and membrane transport from the | | | | | |
| | | outer nuclear membrane to the plasma membrane | | | | | |
| Y75B8A.24 | CAA22108.1 | Y75B8A.24 Member of the phosphatidylinositol 3- and | 181 | 28% | 44% | 146 | 3e−08 |
| | | 4-kinase and phosphoinositide 3-kinase family | | | | | |
| | | accessory domain (PIK domain) containing families, has | | | | | |
| | | moderate similarity to phosphatidylinositol 4-kinase | | | | | |
| | | catalytic alpha peptide (rat Pik4ca) | | | | | |
| age-1 | CAA91377.2 | age-1; daf-23; B0334.8 Aging alteration 1, protein | 280 | 24% | 42% | 147 | 1e−07 |
| | | involved in dauer larva formation, longevity, fertility, | | | | | |
| | | thermotolerance, response to pathogenic bacteria, and | | | | | |
| | | adult motility | | | | | |
| F39B1.1 | CAA93776.1 | F39B1.1 Member of the phosphatidylinositol 3- and 4- | 274 | 24% | 42% | 143 | 3e−07 |
| | | kinase and phosphoinositide 3-kinase family accessory | | | | | |
| | | domain families, contains C2, phosphoinositide 3-kinase | | | | | |
| | | C2, and phox domains and a ubiquitin interaction motif, | | | | | |
| | | has low similarity to human PIK3C2A | | | | | |
| F35H12.4 | AAK39229.1 | F35H12.4 Member of the phosphatidylinositol 3- and 4- | 83 | 36% | 57% | 114 | 1e−04 |
| | | kinase family, has moderate similarity to | | | | | |
| | | phosphatidylinositol 4-kinase beta (human PIK4CB), | | | | | |
| | | which is a wortmannin-sensitive lipid kinase that is | | | | | |
| | | required for the proper organization of the Golgi | | | | | |
| | | complex | | | | | |
| Y48G9A.1 | AAK29920.2 | Y48G9A.1 Protein containing fifteen HEAT repeats, | 316 | 21% | 42% | 112 | 8e−04 |
| | | which appear to function as protein-protein interaction | | | | | |
| | | surfaces, has low similarity to a region of S. cerevisiae | | | | | |
| | | Gcn1p, which is a component of a complex required for | | | | | |
| | | S. cerevisiae Gcn2p activation | | | | | |

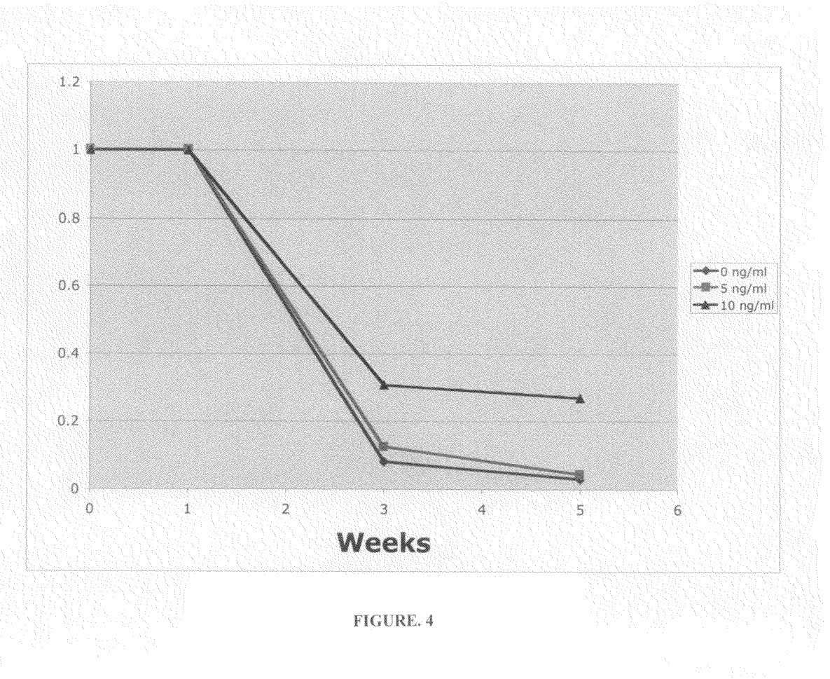

Pharmacological inhibition of the Tor Pathway Extends Chronological Life Span. Rapamycin is a pharmacological agent known to inhibit TOR signaling and thus mimic many aspects of caloric restriction. Given the genetic data implicating the TOR pathway in life span extension, the treatment of cells with rapamycin would extend life span. Doses of 0, 5, or 10 ng/ml of rapamycin to wild-type cells and observed a significant life span increase in cells treated with 10 ng/ml relative to vehicle-treated control cells (FIG. 4). This evidence provides strong support for the role of diminished TOR signaling in life span extension.

Nutrient Partitioning and Life Span. In an organism's natural environment it is likely to encounter repeated periods of plentiful nutrients, followed by times of scarcity and starvation. This “Boom and Bust” cyclical pattern in nature has forced organisms to have different ways of governing growth and reproduction in the face of starvation. When nutrients are available, it is to the organism's advantage to grow and reproduce; conversely when nutrients are limited, it makes sense to turn down reproduction and growth, and up-regulate stress response genes.

Biologists have known that restriction of caloric intake can extend life span in diverse species—including yeast—for many years, however attempts to identify the molecular mechanism(s) involved in this life span extension have been unsuccessful. It is especially interesting to us that many phenotypes induced by caloric restriction are similarly affected when Tor signaling is abrogated.

Several deletions that increase life span (e.g., the permeases) reduce capacity to take up amino acid or other nitrogen rich nutrients such as ammonium. Another class of long-lived mutants we identified (the ribosomal subunits) are involved in incorporating amino acids into proteins, and a third class (the transcription factors and signaling proteins) are involved in coordinating gene expression in response to nutrients. Given the interrelated nature of these proteins, their expression profiles, and their normal role in up-regulating growth and cell division, it is likely that deletion of these proteins tips the energy balance away from growth and reproduction, towards a program of self-maintenance and repair.

These genetic programs include free-radical scavenging enzymes, and proteins involved in turnover of damaged proteins. It is likely that long-lived deletions identified alter the cell's metabolism towards a program induced by starvation, and shunt cellular energy toward these self-maintenance programs.

Tor Mutants and Their Significance for Life Span Extension. Abrogation of Tor-related signaling has been observed to extend life span in C. elegans and D. melanogaster, yet the molecular mechanism for this extension remains unelucidated. It is known however, that Tor is highly conserved in function and amino acid sequence from yeast to humans. Biochemical studies of Tor indicate that it is a phosphatidyl inositol kinase, and/or protein kinase that is active in the presence of abundant amino acids. Tor communicates this nutrient plenty to the cell by signaling a wide range of cellular processes that include ribosome biogenesis, protein synthesis, and cellular division. This signaling takes place via a signaling network that is currently a very active field of research.

Also, it is very likely that further analysis of some of the other proteins disclosed herein influence life span through the Tor pathway will be useful in pinpointing the molecular mechanism of caloric restriction. Because many of these downstream components are closely conserved in humans, the design of agents attenuating Tor effects by targeting these factors can be a viable strategy for drug development in humans.

Use of the CLS Method for Drug Screening. By its nature, the CLS method can be used to screen large libraries of compounds for life span extending activity. We envision conducting large-scale drug screens in yeast and conducting further testing of successful compounds in other eukaryotic systems. Identification of the biochemical pathway through which a life span-extending drug acts can be greatly facilitated through the use of the deletions identified as long-lived (e.g, the genes disclosed in Table 1). For example a life span extending drug can be tested in combination with long-lived deletions. The drug should provide additional life span extension to deletions strains only if the drug does not act through the pathway in which the deletion occurs.

Molecular Changes Important for Life Span Extension. Currently, a better understanding of the changes that take place in cells deleted for Tor pathway components is needed, and further identification is needed of the crucial subset of these changes that confer extended life span. Decreased general protein translation correlates with increased life span, but is this essential for the extension, and if so, how does this allow the cells to live longer? Damaged proteins are known to accumulate as cells age; ultimately these proteins are thought to overwhelm the cell and lead to death. One possible reason decreasing protein synthesis extends life span is that because of decreased translation in long-lived mutants, the cell's protein degradative machinery is able to maintain clearance of damaged proteins for more time, and thus maintain the health of the cell for longer. Direct measurement of protein half-life in cells will be useful for informing drug discovery efforts and designing optimal genetic manipulations in organisms such as mice.

Aging studies in C. elegans. If a yeast strain lacking a gene exhibits extended mean and/or maximum replicative life span, one can infer that the protein encoded by that gene restricts life span in the single-celled eukaryote. This raises the question of whether decreased function of an orthologous protein will result in prolonged life span in another eukaryotic organism. Thus we will test orthologs of yeast aging genes identified in our genome-wide CLS screen in the nematode C. elegans. Over half of the yeast aging genes identified have orthologs in C. elegans. In some cases, more than one C. elegans ortholog bears significant homology to a yeast aging gene and in these cases we will examine the effects of each potential ortholog.

One (or both) or two approaches will be taken to examine C. elegans genes. We will use RNAi, with the double-stranded RNA delivered to the worms through expression in their bacterial food source E. coli. This approach is used routinely and interfering RNAs specific to most C. elegans genes have already been created. As a second approach, we will generate or obtain worms with inactivating mutations in potential aging genes. Life span studies will be performed in a variety of manners. For the RNAi approach, we will shift worms in the L4 larval stage to E. coli expressing the double-stranded RNA. This will lead to downregulation of gene expression of the potential aging gene after development and thus avoid most developmental defects and possible dauer formation, which complicates aging studies. Alternatively, we will administer the RNAi to adult worms and monitor life span of their progeny. In this case, worms will be exposed to the RNAi both during development and as adults. Finally, we will administer candidate compounds identified in yeast studies to determine their potential effects on aging in worms.

Aging studies in mice. An important question will be to determine whether aging genes identified in yeast also affect life span or health span in mammals. Therefore we will initiate aging studies in mice, where gene knockouts of orthologs of identified yeast/worm aging genes can be created. We will choose a subset of the genes identified in our yeast studies, emphasizing those that also regulate aging in worms or another multicellular model. Given the evolutionary divergence of worms and yeast, we feel strongly that gene sets regulating aging in both organisms, are highly likely to regulate aging in mammals as well.

Experiments will be performed by generating conditional knock-outs of orthologs of yeast and worm aging genes. By flanking the gene in question with lox sites, we can control when the gene is excised by temporal or tissue-specific administration of Cre, an enzyme that excises DNA between two lox sites. Therefore, we can allow mice to undergo fetal development and then generate the gene deletion post-natally by ubiquitous delivery of Cre. This will allow us to avoid developmental defects associated with loss of a candidate aging gene that would impair aging studies. Post-natal administration can be performed in a variety of documented methods including but not limited to the use of a tet-regulated promoter driving expression of Cre present in the germ line of the mouse. Should post-natal administration result in lethality or other phenotypes which preclude aging analysis, we will perform life span analysis in mice heterozygous for the gene in question.

In addition to monitoring mouse life span, we will examine a variety of aging biomarkers including but not limited to changes in cognitive ability, fat mass, strength, as well as alopecia and blood serum levels of a variety of compounds including insulin, IGF, glucose, leptin, DHEA, growth hormone, and molecules diagnostic of immune response. Additionally we will perform gene expression array analysis looking at genome-wide changes in gene expression during the mouse aging process. Changes in expression of many genes known to occur during aging and by monitoring these genes, we can measure rates of aging. By using such a biomarkers, we can (1) determine whether a gene deletion is likely to affect life span prior to the completion of aging studies and (2) monitor changes in health span that occur with age.

Finally, we will examine the effects of prolonged administration of drugs on mouse aging and aforementioned biomarkers of aging. Drugs that are effective in yeast and worms will be chosen for studies in mice.

Gene expression array analysis. We will use genome-wide gene expression array analysis in all three organisms (yeast, worms and mice), as well as potentially on human cells in culture, to (1) determine aging rates and (2) identify downstream targets of aging genes that might underlie delayed aging phenotypes.

In worms, we will monitor changes in genome-wide gene expression in worms lacking aging genes. This analysis may be performed both in young worms and throughout the aging process. In addition, we will examine environmental conditions that extend life span and compounds as described above. Similar experiments will be performed in young and aging mice. Again, this will allow us to monitor the rate of aging by looking at changes in gene expression known to occur during murine aging and to identify critical targets of aging genes.

Polymorphisms and human aging. Loss-of-function mutations resulting in prolonged life span or health span in yeast, worms and mice might be phenocopied by polymorphisms that have arisen in the human population. Thus, we will determine whether aging genes identified in yeast, worms or mice are enriched for particular polymorphisms in unusually old individuals relative to the normal population. An enhancement in the relative proportion of a particular allele of a potential aging gene would provide evidence for the gene regulating in humans.

Each recited range includes all combinations and sub-combinations of ranges, as well as specific numerals contained therein.

All publications and patent applications cited in this specification are herein incorporated by reference in their entirety for all purposes as if each individual publication or patent application were specifically and individually indicated to be incorporated by reference for all purposes.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

What is claimed:

1. A method of slowing the rate of aging of a eukaryote by treating the eukaryote with a bioactive agent that inhibits the TOR pathway. [0010][0011][0014][0021-0023][0029-0030][0032][0037][0040-0042][0049][0051][0057][0075][0092-0093][0098-0099][0157][0160][0018][0449][0525-0542][0322][0343][0400][0419-0426][0446][0448]

2. The method according to claim 1, wherein the bioactive agent is rapamycin, a rapamycin analog or derivative, or pharmaceutically acceptable salt thereof.

3. The method according to claim 1, wherein the eukaryote is a mammal.

4. The method according to claim 2, wherein the eukaryote is a mammal.

5. The method according to claim 2, wherein the eukaryote is a human.

6. The method according to claim 3, wherein the eukaryote is a human.

7. A method of treating a subject with a TOR pathway inhibitor to slow or prevent the onset of an age related disease. [0010][0011][0014][0021-0023][0029-0030][0032][0037][0040-0042][0049][0051][0057][0075][0092-0093][0098-0099][0157][0160][0018][0449][0525-0542][0322][0343][0400][0419-0426][0446][0448]

8. The method according to claim 7, wherein the disease is cancer.

9. The method according to claim 7, wherein the disease is heart disease.

10. The method according to claim 7, wherein the disease is Alzheimer's disease.

11. The method according to claim 7, wherein the disease is Pick's disease.

12. The method according to claim 7, wherein the disease is Huntington's disease.

13. The method according to claim 7, wherein the disease is Parkinson's disease.

14. The method according to claim 7, wherein the disease is adult onset myotonic dystrophy.

15. The method according to claim 7, wherein the disease is multiple sclerosis.

16. The method according to claim 7, wherein the disease is adult onset leukodystrophy.

17. The method according to claim 7, wherein the disease is diabetes mellitus.

18. A method for inhibiting a gene or gene product or ortholog thereof of a gene or gene product of table 1 or table 2, comprising administering rapamycin, a rapamycin analog or derivative, or pharmaceutically acceptable salt thereof to a eukaryote in order to slow the rate of aging of the eukaryote. [0010][0011][0014][0021-0023][0029-0030][0032][0037][0040-0042][0049][0051][0057][0075][0092-0093][0098-0099][0157][0160][0018][0449][0525-0542][0322][0343][0400][0419-0426][0446][0448]

19. The method of claim 18 wherein the eukaryote is a mammal.

20. The method of claim 19 wherein the eukaryote is a human.

21. A method of preventing the onset of an age-related disease by administering a bioactive agent that inhibits the TOR pathway. [0010][0011][0014][0021-0023][0029-0030][0032][0037][0040-0042][0049][0051][0057][0075][0092-0093][0098-0099][0157][0160][0018][0449][0525-0542][0322][0343][0400][0419-0426][0446][0448]

22. The method according to claim 21, wherein the disease is cancer.

23. The method according to claim 21, wherein the disease is heart disease.

24. The method according to claim 21, wherein the disease is Alzheimer's disease.

25. The method according to claim 21, wherein the disease is Pick's disease.

26. The method according to claim 21, wherein the disease is Huntington's disease.

27. The method according to claim 21, wherein the disease is Parkinson's disease.

28. The method according to claim 21, wherein the disease is adult onset myotonic dystrophy.

29. The method according to claim 21, wherein the disease is multiple sclerosis.

30. The method according to claim 21, wherein the disease is adult onset leukodystrophy.

31. The method according to claim 21, wherein the disease is diabetes mellitus.

32. A method of slowing the progression of an age-related disease by administering a bioactive agent that inhibits the TOR pathway.

33. The method according to claim 32, wherein the disease is heart disease.

34. The method according to claim 32, wherein the disease is Alzheimer's disease.

35. The method according to claim 32, wherein the disease is Pick's disease.

36. The method according to claim 32, wherein the disease is Huntington's disease.

37. The method according to claim 32, wherein the disease is Parkinson's disease.

38. The method according to claim 32, wherein the disease is adult onset myotonic dystrophy.

39. The method according to claim 32, wherein the disease is multiple sclerosis.

40. The method according to claim 32, wherein the disease is adult onset leukodystrophy.

41. The method according to claim 32, wherein the disease is diabetes mellitus.
